(19) United States
(12) Patent Application Publication
(10) Pub. No.: US 2023/0129788 A1
(43) Pub. Date: Apr. 27, 2023

(54) GLP-1/GLP-2 DUAL AGONISTS

(71) Applicant: Zealand Pharma A/S, Søborg (DK)

(72) Inventors: Jonathan Griffin, Søborg (DK); Per-Olof Eriksson, Søborg (DK); Wayne Russell, Søborg (DK); Jolanta Skarbaliene, Søborg (DK)

(21) Appl. No.: 17/906,888

(22) PCT Filed: Mar. 29, 2021
(86) PCT No.: PCT/EP2021/058189

§ 371 Date: Sep. 21, 2022

(30) Foreign Application Priority Data
Mar. 30, 2020 (DK) 20166864.7

(51) Int. Cl. A61K 38/26

(52) U.S. Cl. CPC A61K 38/26

(57) ABSTRACT

The invention relates to a composition comprising a GLP-1/GLP-2 dual agonist for use in the treatment of a patient receiving parenteral nutrition.

### FIELD OF THE INVENTION

The present invention relates to a composition comprising a GLP-1/GLP-2 dual agonist for use in the treatment of a patient receiving parenteral nutrition.

### BACKGROUND TO THE INVENTION

Providing parenteral nutrition (PN) is the process of providing nutritional needs intravenously. It remains an essential lifesaving therapy in individuals where regular enteral nutrition cannot be provided. However, despite widespread use, enthusiasm is tempered due to significant side effects of gut atrophy and hepatic injury.

There is a need in the art for therapeutic options for patients who are receiving parenteral nutrition.

### SUMMARY OF THE INVENTION

The present inventors have surprisingly found that administering a GLP-1/GLP-2 dual agonist to a patient who is receiving parenteral nutrition can ameliorate side effects associated with parenteral nutrition.

Broadly, the present invention relates to compounds which have agonist activity at the GLP-1 (glucagon-like peptide 1) and GLP-2 (glucagon-like peptide 2) receptors for use in the treatment of patients who are receiving parenteral nutrition.

In one aspect the invention provides composition comprising a GLP-1/GLP-2 dual agonist for use in the treatment of a patient receiving parenteral nutrition.

In one aspect the composition for use according to the invention may prevent or treat malabsorption, ulcers, short-bowel syndrome, cul-de-sac syndrome, inflammatory bowel disease, irritable bowel syndrome, pouchitis, celiac sprue, tropical sprue, hypogammaglobulinemic sprue, mucositis induced by chemotherapy or radiation therapy, diarrhea induced by chemotherapy or radiation therapy, low grade inflammation, metabolic endotoxemia, necrotising enterocolitis, primary biliary cirrhosis, hepatitis, fatty liver disease, or gastrointestinal side-effects of inflammatory conditions.

In one aspect said patient has short bowel syndrome.

In one aspect said patient has intestinal insufficiency or failure.

In one aspect said patient has hepatic injury/impairment or insufficiency.

In one aspect the GLP-1/GLP-2 dual agonist is a peptide.

In one aspect said GLP-1/GLP-2 dual agonist is a compound represented by the formula:

R¹—X*-U—R²

wherein:

R¹ is hydrogen (Hy), C₁₋₄ alkyl (e.g. methyl), acetyl, formyl, benzoyl or trifluoroacetyl;

R² is NH₂ or OH;

X* is a peptide of formula I:

H-X2-EG-X5-F-X7-X8-E-X10-X11-TIL-X15-X16-X17-A-X19-X20-X21-FI-X24-WL-X27-X28-X29-KIT-X33 (I)

wherein:

X2 is Aib or G

X5 is T or S;

X7 is T or S;

X8 is S, E or D;

X10 is L, M, V or ψ;

X11 is A, N or S;

X15 is D or E;

X16 is G, E, A or ψ;

X17 is Q, E, K, L or ψ;

X19 is A, V or S;

X20 is R, K or ψ;

X21 is D, L or E;

X24 is A, Nor S;

X27 is I, Q, K, H or Y;

X28 is Q, E, A, H, Y, L, K, R or S;

X29 is H, Y, K or Q;

X33 is D or E;

U is absent or a sequence of 1-15 residues each independently selected from K, k, E, A, T, I, L and ψ;

the molecule contains one and only one 4), wherein 4) is a residue of K, k, R, Orn, Dap or Dab in which the side chain is conjugated to a substituent having the formula Z¹— or Z¹—Z²—, wherein

Z¹— is CH₃—(CH₂)₁₀₋₂₂—(CO)— or HOOC—(CH₂)₁₀₋₂₂—(CO)—; and

—Z²— is selected from —Z^{S1}—, —Z^{S1}—Z^{S2}—, —Z^{S2}—Z^{S1}—, —Z^{S2}—, —Z^{S3}—, —Z^{S1}Z^{S3}—, —Z^{S2}Z^{S3}—, —Z^{S3}Z^{S1}—, —Z^{S3}Z^{S2}—, —Z^{S1}Z^{S2}Z^{S3}—, —Z^{S1}Z^{S3}Z^{S2}—, —Z^{S2}Z^{S1}Z^{S3}—, —Z^{S2}Z^{S3}Z^{S1}—, —Z^{S3}Z^{S1}Z^{S2}—, —Z^{S3}Z^{S2}Z^{S1}—, —Z^{S2}Z^{S3}Z^{S2}—

wherein

Z^{S1} is isoGlu, β-Ala, isoLys, or 4-aminobutanoyl;

Z^{S2} is -(Peg3)ₘ- where m is 1, 2, or 3; and

—Z^{S3}— is a peptide sequence of 1-6 amino acid units independently selected from the group consisting of A, L, S, T, Y, Q, D, E, K, k, R, H, F and G;

and wherein at least one of X5 and X7 is T;

or a pharmaceutically acceptable salt or solvate thereof.

The various amino acid positions in peptide X* of the formulae provided here are numbered according to their linear position from N- to C-terminus in the amino acid chain.

In the present context, β-Ala and 3-Aminopropanoyl are used interchangeably.

Dual agonists having aspartic acid (Asp, D) at position 3 and glycine (Gly) in position 4 can be very potent agonists at the GLP-1 and GLP-2 receptors. However, this combination of substitutions results in compounds which are unstable and may not be suitable for long term storage in aqueous solution. Without wishing to be bound by theory, it is believed that the Asp at position 3 may isomerise to iso-Asp via a cyclic intermediate formed between the carboxylic acid functional group of its side chain and the backbone nitrogen atom of the residue at position 4.

It has now been found that molecules having glutamic acid (Glu, E) at position 3 instead of Asp are much less susceptible to such reactions and hence may be considerably more stable when stored in aqueous solution. However, replacement of Asp with Glu at position 3 in molecules having a lipophilic substituent in the middle portion of the peptide (e.g. at or near to positions 16 and 17) tends to reduce the potency at one or both of the GLP-2 receptor and the GLP-1 receptor, even though Glu is present at position 3 of the native GLP-1 molecule. Simultaneously incorporating a Thr residue at one or both of positions 5 and 7 appears to compensate for some or all of the lost potency. It is believed that further improvements in potency are also provided by incorporation of His (H), Tyr (Y), Lys (K) or Gln (Q) at position 29 instead of the Gly (G) and Thr (T) residues present in wild type human GLP-1 and 2 respectively.

In some embodiments of formula I:

X2 is Aib or G

X5 is T or S;

X7 is T or S;

X8 is S;

X10 is L or ψ;

X11 is A or S;

X15 is D or E;

X16 is G, E, A or ψ;

X17 is Q, E, K, L or ψ;

X19 is A or S;

X20 is R or ψ;

X21 is D, L or E;

X24 is A;

X27 is I, Q, K, or Y;

X28 is Q, E, A, H, Y, L, K, R or S;

X29 is H, Y or Q; and

X33 is D or E.

Where ψ is not at X16 or X17, it may be desirable that X16 is E and X17 is Q.

In some embodiments, X11 is A and X15 is D. In other embodiments, X11 is S and X15 is

E. In further embodiments, X11 is A and X15 is E.

In some embodiments, X27 is I.

In some embodiments, X29 is H. In certain of these embodiments, X28 is A and X29 is H, or X28 is E and X29 is H.

In some embodiments, X29 is Q and optionally X27 is Q.

In some embodiments, the residues at X27-X29 have a sequence selected from:

IQH;

IEH

IAH;

IHH;

IYH;

ILH;

IKH;

IRH;

ISH;

QQH;

YQH;

KQH;

IQQ;

IQY;

IQT; and

IAY.

In some embodiments, X* is a peptide of formula II:

H-X2-EG-X5-F-X7-SELATILD-X16-X17-AAR-X21-FIAWLI-X28-X29-KITD (II)

wherein:

X2 is Aib or G

X5 is T or S;

X7 is T or S;

X16 is G or ψ;

X17 is Q, E, K, L or ψ;

X21 is D or L;

X28 is Q, E, A, H, Y, L, K, R or S;

X29 is H, Y or Q;

In some embodiments of Formula I or Formula II, X16 is ψ and X17 is Q, E, K or L. For example, X17 may be Q, or X17 may be selected from E, K and L. In other embodiments,

X16 is G and X17 is ψ.

It may be desirable that X21 is D.

X28 may be selected from Q, E and A, e.g. it may be Q or E. In some residue combinations,

Q may be preferred. In others, E may be preferred, including but not limited to when X16 is G and X17 is ψ. Alternatively, X28 may be selected from A, H, Y, L, K, R and S.

X* may be a peptide of formula III:

H[Aib]EG-X5-F-X7-SE-X10-ATILD-X16-X17-AA-X20-X21-FIAWLI-X28-X29-KITD (III)

wherein:

X5 is T or S;

X7 is T or S;

X10 is L or ψ;

X16 is G, E, A or ψ;

X17 is Q, E, K, L or ψ;

X20 is R or ψ;

X21 is D or L;

X28 is E, A or Q;

X29 is H, Y or Q;

and at least one of X5 and X7 is T.

X* may be a peptide of formula IV:

H[Aib]EG-X5-F-X7-SELATILD-X16-X17-AAR-X21-FIAWLI-X28-X29-KITD (IV)

wherein:

X5 is T or S;

X7 is T or S;

X16 is G or ψ;

X17 is E, K, L or ψ;

X21 is D or L;

X28 is E or A;

X29 is H, Y or Q;

and at least one of X5 and X7 is T.

In some embodiments of any of formulae I to IV, X16 is ψ and X17 is E, K or L.

In other embodiments of formula I to IV, X16 is G and X17 is ψ.

In either case, the following combinations of residues may also be included:

X21 is D and X28 is E;

X21 is D and X28 is A;

X21 is L and X28 is E;

X21 is L and X28 is A.

X* may be a peptide of formula V:

H[Aib]EG-X5-F-X7-SELATILD-ψ-QAARDFIAWLI-X28-X29-KITD (V)

wherein

X5 is T or S;

X7 is T or S;

X28 is Q, E, A, H, Y, L, K, R or S, e.g. Q, E, A, H, Y or L;

X29 is H, Y or Q;

and at least one of X5 and X7 is T.

In some embodiments of formula III, X28 is Q or E. In some embodiments of formula III, X28 is Q. In other embodiments, X28 is A, H, Y, L, K, R or S, e.g. A, H, Y or L.

In any of the formulae or embodiments described above, the dual agonist contains one of the following combinations of residues:

X5 is S and X7 is T;

X5 is T and X7 is S;

X5 is T and X7 is T.

It may be preferred that X5 is S and X7 is T, or X5 is T and X7 is T.

In any of the formulae or embodiments described above, it may be desirable that X29 is H.

In some embodiments, 4⁴ is a Lys residue whose side chain is conjugated to the substituent Z¹— or Z¹—Z²—.

In some embodiments, Z¹—, alone or in combination with —Z²—, is dodecanoyl, tetradecanoyl, hexadecanoyl, octadecanoyl or eicosanoyl.

In some embodiments, Z¹—, alone or in combination with —Z²—, is:

13-carboxytridecanoyl, i.e. HOOC—(CH₂)₁₂—(CO)—;

15-carboxypentadecanoyl, i.e. HOOC—(CH₂)₁₄—(CO)—;

17-carboxyheptadecanoyl, i.e. HOOC—(CH₂)₁₆—(CO)—;

19-carboxynonadecanoyl, i.e. HOOC—(CH₂)₁₈—(CO)—; or

21-carboxyheneicosanoyl, i.e. HOOC—(CH₂)₂₀—(CO)—.

In some embodiments Z² is absent.

In some embodiments, Z² comprises Z^{S1}alone or in combination with Z^{S2} and/or Z^{S3}.

In such embodiments:

—Z^{S1}— is isoGlu, β-Ala, isoLys, or 4-aminobutanoyl;

—Z^{S2}—, when present, is -(Peg3)ₘ- where m is 1, 2, or 3; and

—Z^{S3}— is a peptide sequence of 1-6 amino acid units independently selected from the group consisting of A, L, S, T, Y, Q, D, E, K, k, R, H, F and G, such as the peptide sequence KEK.

Z² may have the formula —Z^{S1}—Z^{S3}—Z^{S2}—, where Z^{S1} is bonded to Z¹ and Z^{S2} is bonded to the side chain of the amino acid component of ψ.

Thus, in some embodiments, —Z²— is:

isoGlu(Peg 3)₀₋₃;

β-Ala(Peg 3)₀₋₃;

isoLys(Peg3)₀₋₃; or

4-aminobutanoyl(Peg3)₀₋₃.

In further embodiments, —Z²— is:

isoGlu-KEK-(Peg3)₀₋₃.

Specific examples of the substituent Z¹—Z²— are set out below. In some embodiments, Z¹—Z²— is [17-carboxy-heptadecanoyl]-isoGlu. For example, ψ may be K([17-carboxy-heptadecanoyl]-isoGlu). In some embodiments, Z¹—Z²— is:

[17-Carboxy-heptadecanoyl]-isoGlu-KEK-Peg3-;

[17-carboxy-heptadecanoyl]-isoGlu-Peg3-;

[19-Carboxy-nonadecanoyl]-isoGlu-;

[19-Carboxy-nonadecanoyl]-isoGlu-KEK-;

[19-Carboxy-nonadecanoyl]-isoGlu-KEK-Peg3-;

[19-carboxy-nonadecanoyl]-isoGlu-KEK-Peg3-Peg3-;

[19-carboxy-nonadecanoyl]-isoGlu-Peg3-Peg3-;

[19-carboxy-nonadecanoyl]-isoLys-Peg3-Peg3-Peg3-;

[Hexadecanoyl]-βAla-;

[Hexadecanoyl]-isoGlu-; or

Octadecanoyl-.

For example, ψ may be:

K([17-Carboxy-heptadecanoyl]-isoGlu-KEK-Peg3);

K([17-carboxy-heptadecanoyl]-isoGlu-Peg3);

K([19-Carboxy-nonadecanoyl]-isoGlu);

K([19-Carboxy-nonadecanoyl]-isoGlu-KEK);

K([19-Carboxy-nonadecanoyl]-isoGlu-KEK-Peg3);

K([19-carboxy-nonadecanoyl]-isoGlu-KEK-Peg3-Peg3);

K([19-carboxy-nonadecanoyl]-isoGlu-Peg3-Peg3);

K([19-carboxy-nonadecanoyl]-isoLys-Peg3-Peg3-Peg3);

K([Hexadecanoyl]-βAla-;

K([Hexadecanoyl]-isoGlu); or

K(Octadecanoyl).

When present, U represents a peptide sequence of 1-15 residues each independently selected from K (i.e. L-lysine), k (i.e. D-lysine) E (Glu), A (Ala), T (Thr), I (Ile), L (Leu) and ψ. For example, U may be 1-10 amino acids in length, 1-7 amino acids in length, 3-7 amino acids in length, 1-6 amino acids in length, or 3-6 amino acids in length.

Typically U includes at least one charged amino acid (K, k or E) and preferably two or more charged amino acids. In some embodiments it includes at least 2 positively charged amino acids (K or k), or at least 1 positively charged amino acid (K or k) and at least one negatively charged amino acid (E). In some embodiments, all amino acid residues of U (except for ψ, if present) are charged. For example, U may be a chain of alternately positively and negatively charged amino acids.

In certain embodiments, U comprises residues selected only from K, k, E and ψ.

In certain embodiments, U comprises residues selected only from K, k, and ψ.

When U comprises only lysine residues (whether K or k), all residues may have an L-configuration or all may have a D-configuration. Examples include K₁₋₁₅, K₁₋₁₀ and K₁₋₇, e.g., K₃, K₄, K₅, K₆ and K₇, especially K₅ and K₆. Further examples include k₁₋₁₅, k₁₋₁₀ and k₁₋₇, e.g. k₃, k₄, k₅, k₆ and k₇, especially ks and k₆.

Further examples of peptide sequences U include KEK, EKEKEK, EkEkEk, AKAAEK, AKEKEK and ATILEK.

In any case, one of those residues may be exchanged for ψ. Where the sequence U contains a residue ψ, it may be desirable that the C-terminal residue of U is ψ. Thus, further examples of sequences U include K₁₋₁₄-ψ, K₁₋₉-ψ, and K₁₋₆-ψ, e.g., K₂-ψ, K₃-ψ, K₄-ψ, K₅-ψ and K₆-ψ, especially K₄-ψ and K₅.-ψ. Yet further examples include k₁₋₁₄-ψ, k₁₋₉-ψ, and k₁₋₆-ψ, e.g. k₂-ψ, k₃-ψ, k₄-ψ, k₅-ψ and k₆-ψ especially k₄-ψ and k₅-ψ. Yet further examples include KEψ, EKEKEψ, EkEkEψ AKAAEψ, AKEKEψ and ATILEψ.

In some embodiments, U is absent.

In some embodiments, R¹ is Hy and/or R² is OH.

The peptide X* or the peptide X*-U may have the sequence:

H[Aib]EGTFSSELATILDΨEAARDFIAWLIEHKITD;
H[Aib]EGSFTSELATILDΨEAARDFIAWLIEHKITD;
H[Aib]EGTFTSELATILDΨEAARDFIAWLIEHKITD;
H[AIb]EGTFSSELATILDΨKAARDFIAWLIEHKITD;
H[Aib]EGSFTSELATILDΨKAARDFIAWLIEHKITD;
H[Aib]EGTFTSELATILDΨKAARDFIAWLIEHKITD;
H[Aib]EGTFSSELATILDGΨAARDFIAWLIEHKITD;
H[Aib]EGSFTSELATILDGΨAARDFIAWLIEHKITD;
H[Aib]EGTFTSELATILDGΨAARDFIAWLIEHKITD;
H[Aib]EGTFSSELATILDΨLAARDFIAWLIEHKITD;
H[Aib]EGSFTSELATILDΨLAARDFIAWLIEHKITD;
H[Aib]EGTFTSELATILDΨLAARDFIAWLIEHKITD;
H[Aib]EGTFSSELATILDΨLAARDFIAWLIAHKITD;
H[Aib]EGSFTSELATILDΨLAARDFIAWLIAHKITD;
H[Aib]EGTFTSELATILDΨLAARDFIAWLIAHKITD;
H[Aib]EGTFTSELATILDΨEAARLFIAWLIEHKITD;
H[Aib]EGTFSSELATILDΨQAARDFIAWLIQHKITD;
H[Aib]EGSFTSELATILDΨQAARDFIAWLIQHKITD;
H[Aib]EGTFTSELATILDΨQAARDFIAWLIQHKITD;
H[Aib]EGTFSSELATILDΨQAARDFIAWLIEHKITD;
H[Aib]EGTFSSELATILDΨQAARDFIAWLIAHKITD;
H[Aib]EGSFTSELATILDΨQAARDFIAWLIAHKITD;
H[Aib]EGTFTSELATILDΨQAARDFIAWLIAHKITD;
H[Aib]EGSFTSELATILDΨQAARDFIAWLIEHKITD;
H[Aib]EGTFTSELATILDΨQAARDFIAWLIEHKITD;
H[Aib]EGSFTSELATILDΨQAARDFIAWLIHHKITD;
H[Aib]EGSFTSELATILDΨQAARDFIAWLIYHKITD;
H[Aib]EGSFTSELATILDΨQAARDFIAWLILHKITD;
H[Aib]EGSFTSELATILDΨQAARDFIAWLIKHKITD;
H[Aib]EGSFTSELATILDΨQAARDFIAWLIRHKITD;
H[Aib]EGSFTSELATILDΨQAARDFIAWLISHKITD
H[Aib]EGSFTSELATILDΨQAARDFIAWLQQHKITD;
H[Aib]EGSFTSELATILDΨQAARDFIAWLYQHKITD;
H[Aib]EGSFTSELATILDΨQAARDFIAWLKQHKITD;
H[Aib]EGSFTSELATILDΨQAARDFIAWLIQQKITD;
H[Aib]EGSFTSELATILDΨQAARDFIAWLIQYKITD;
H[Aib]EGTFSSELSTILEWQASREFIAWLIAYKITE;
H[Aib]EGTFSSELATILDEQAARDFIAWLIAHKITDkkkkkΨ;
H[Aib]EGTFTSELATILDEQAARDFIAWLIAHKITDkkkkkΨ;
H[Aib]EGSFTSELATILDEQAARDFIAWLIEHKITDkkkkkΨ;
H[Aib]EGSFTSEΨATILDEQAARDFIAWLIEHKITD;
H[Aib]EGSFTSELATILEGΨAARDFIAWLIEHKITD;
H[Aib]EGSFTSELATILDEQAAΨDFIAWLIEHKITD;
H[Aib]EGTFTSELATILDEQAAΨDFIAWLIEHKITD;
H[Aib]EGTFTSEΨATILDEQAARDFIAWLIEHKITD;
H[Aib]EGSFTSELATILDΨAARDFIAWLIEHKITD;
or
H[Aib]EGSFTSELATILDAKAAΨDFIAWLIEHKITD.

The peptide X* or the peptide X*-U may have the sequence:

H[Aib]EGTFSSELATILD[K*]EAARDFIAWLIEHKITD;
H[Aib]EGSFTSELATILD[K*]EAARDFIAWLIEHKITD;
H[Aib]EGTFTSELATILD[K*]EAARDFIAWLIEHKITD;
H[Aib]EGTFSSELATILD[K*]KAARDFIAWLIEHKITD;
H[Aib]EGSFTSELATILD[K*]KAARDFIAWLIEHKITD;
H[Aib]EGTFTSELATILD[K*]KAARDFIAWLIEHKITD;
H[Aib]EGTFSSELATILDG[K*]AARDFIAWLIEHKITD;
H[Aib]EGSFTSELATILDG[K*]AARDFIAWLIEHKITD;
H[Aib]EGTFTSELATILDG[K*]AARDFIAWLIEHKITD;
H[Aib]EGTFSSELATILD[K*]LAARDFIAWLIEHKITD;
H[Aib]EGSFTSELATILD[K*]LAARDFIAWLIEHKITD;
H[Aib]EGTFTSELATILD[K*]LAARDFIAWLIEHKITD;
H[Aib]EGTFSSELATILD[K*]LAARDFIAWLIAHKITD;
H[Aib]EGSFTSELATILD[K*]LAARDFIAWLIAHKITD;
H[Aib]EGTFTSELATILD[K*]LAARDFIAWLIAHKITD;
H[Aib]EGTFTSELATILD[K*]EAARLFIAWLIEHKITD;
H[Aib]EGTFSSELATILD[K*]QAARDFIAWLIQHKITD;
H[Aib]EGSFTSELATILD[K*]QAARDFIAWLIQHKITD;
H[Aib]EGTFTSELATILD[K*]QAARDFIAWLIQHKITD;
H[Aib]EGTFSSELATILD[K*]QAARDFIAWLIEHKITD;
H[Aib]EGTFSSELATILD[K*]QAARDFIAWLIAHKITD;
H[Aib]EGSFTSELATILD[K*]QAARDFIAWLIAHKITD;
H[Aib]EGTFTSELATILD[K*]QAARDFIAWLIAHKITD;
H[Aib]EGSFTSELATILD[K*]QAARDFIAWLIEHKITD;
H[Aib]EGTFTSELATILD[K*]QAARDFIAWLIEHKITD;
H[Aib]EGSFTSELATILD[K*]QAARDFIAWLIHHKITD;
H[Aib]EGSFTSELATILD[K*]QAARDFIAWLIYHKITD;
H[Aib]EGSFTSELATILD[K*]QAARDFIAWLILHKITD;
H[Aib]EGSFTSELATILD[K*]QAARDFIAWLIKHKITD;
H[Aib]EGSFTSELATILD[K*]QAARDFIAWLIRHKITD;
H[Aib]EGSFTSELATILD[K*]QAARDFIAWLISHKITD;
H[Aib]EGSFTSELATILD[K*]QAARDFIAWLQQHKITD;
H[Aib]EGSFTSELATILD[K*]QAARDFIAWLYQHKITD;
H[Aib]EGSFTSELATILD[K*]QAARDFIAWLKQHKITD;
H[Aib]EGSFTSELATILD[K*]QAARDFIAWLIQQKITD;
H[Aib]EGSFTSELATILD[K*]QAARDFIAWLIQYKITD;
H[Aib]EGTFSSELSTILE[K*]QASREFIAWLIAYKITE;
H[Aib]EGTFSSELATILDEQAARDFIAWLIAHKITDkkkkk[k*];
H[Aib]EGTFTSELATILDEQAARDFIAWLIAHKITDkkkkk[k*];
H[Aib]EGSFTSELATILDEQAARDFIAWLIEHKITDkkkkk[k*];
H[Aib]EGSFTSE[K*]ATILDEQAARDFIAWLIEHKITD;
H[Aib]EGSFTSELATILEG[K*]AARDFIAWLIEHKITD;
H[Aib]EGSFTSELATILDEQAA[K*]DFIAWLIEHKITD;
H[Aib]EGTFTSELATILDEQAA[K*]DFIAWLIEHKITD;
H[Aib]EGTFTSE[K*]ATILDEQAARDFIAWLIEHKITD;
H[Aib]EGSFTSELATILDA[K*]AARDFIAWLIEHKITD;
or
H[Aib]EGSFTSELATILDAKAA[K*]DFIAWLIEHKITD;

wherein K* or k* indicates an L or D lysine residue respectively in which the side chain is conjugated to the substituent Z¹— or Z¹Z²—.

For example, the peptide X* or the peptide X*-U may have the sequence:

H[Aib]EGTFSSELATILD[K([17-carboxy-heptadecanoyl]-isoGlu)]EAARDFIAWLIEHKITD;
H[Aib]EGSFTSELATILD[K([17-carboxy-heptadecanoyl]-isoGlu)]EAARDFIAWLIEHKITD;
H[Aib]EGTFTSELATILD[K([17-carboxy-heptadecanoyl]-isoGlu)]EAARDFIAWLIEHKITD;
H[Aib]EGTFSSELATILD[K([17-carboxy-heptadecanoyl]-isoGlu)]KAARDFIAWLIEHKITD;
H[Aib]EGSFTSELATILD[K([17-carboxy-heptadecanoyl]-isoGlu)]KAARDFIAWLIEHKITD;
H[Aib]EGTFTSELATILD[K([17-carboxy-heptadecanoyl]-isoGlu)]KAARDFIAWLIEHKITD;
H[Aib]EGTFSSELATILDG[K([17-carboxy-heptadecanoyl]-isoGlu)]AARDFIAWLIEHKITD;
H[Aib]EGSFTSELATILDG[K([17-carboxy-heptadecanoyl]-isoGlu)]AARDFIAWLIEHKITD;
H[Aib]EGTFTSELATILDG[K([17-carboxy-heptadecanoyl]-isoGlu)]AARDFIAWLIEHKITD;
H[Aib]EGTFSSELATILD[K([17-carboxy-heptadecanoyl]-isoGlu)]LAARDFIAWLIEHKITD;
H[Aib]EGSFTSELATILD[K([17-carboxy-heptadecanoyl]-isoGlu)]LAARDFIAWLIEHKITD;
H[Aib]EGTFTSELATILD[K([17-carboxy-heptadecanoyl]-isoGlu)]LAARDFIAWLIEHKITD;
H[Aib]EGTFSSELATILD[K([17-carboxy-heptadecanoyl]-isoGlu)]LAARDFIAWLIAHKITD;
H[Aib]EGSFTSELATILD[K([17-carboxy-heptadecanoyl]-isoGlu)]LAARDFIAWLIAHKITD;
H[Aib]EGTFTSELATILD[K([17-carboxy-heptadecanoyl]-isoGlu)]LAARDFIAWLIAHKITD;
H[Aib]EGTFTSELATILD[K([17-carboxy-heptadecanoyl]-isoGlu)]EAARLFIAWLIEHKITD;
H[Aib]EGTFSSELATILD[K([17-carboxy-heptadecanoyl]-isoGlu)]QAARDFIAWLIQHKITD;
H[Aib]EGSFTSELATILD[K([17-carboxy-heptadecanoyl]-isoGlu)]QAARDFIAWLIQHKITD;
H[Aib]EGTFTSELATILD[K([17-carboxy-heptadecanoyl]-isoGlu)]QAARDFIAWLIQHKITD;
H[Aib]EGTFSSELATILD[K([17-carboxy-heptadecanoyl]-isoGlu)]QAARDFIAWLIEHKITD;
H[Aib]EGTFSSELATILD[K([17-carboxy-heptadecanoyl]-isoGlu)]QAARDFIAWLIAHKITD;
H[Aib]EGSFTSELATILD[K([17-carboxy-heptadecanoyl]-isoGlu)]QAARDFIAWLIAHKITD;
H[Aib]EGTFTSELATILD[K([17-carboxy-heptadecanoyl]-isoGlu)]QAARDFIAWLIAHKITD;
H[Aib]EGSFTSELATILD[K([17-carboxy-heptadecanoyl]-isoGlu)]QAARDFIAWLIEHKITD;
H[Aib]EGTFTSELATILD[K([17-carboxy-heptadecanoyl]-isoGlu)]QAARDFIAWLIEHKITD;
H[Aib]EGSFTSELATILD[K([17-carboxy-heptadecanoyl]-isoGlu)]QAARDFIAWLIHHKITD;
H[Aib]EGSFTSELATILD[K([17-carboxy-heptadecanoyl]-isoGlu)]QAARDFIAWLIYHKITD;
H[Aib]EGSFTSELATILD[K([17-carboxy-heptadecanoyl]-isoGlu)]QAARDFIAWLILHKITD;
H[Aib]EGSFTSELATILD[K([17-carboxy-heptadecanoyl]-isoGlu)]QAARDFIAWLIKHKITD;
H[Aib]EGSFTSELATILD[K([17-carboxy-heptadecanoyl]-isoGlu)]QAARDFIAWLIRHKITD;
H[Aib]EGSFTSELATILD[K([17-carboxy-heptadecanoyl]-isoGlu)]QAARDFIAWLISHKITD;
H[Aib]EGSFTSELATILD[K([Hexadecanoyl]-pAla)]QAARDFIAWLQQHKITD;
H[Aib]EGSFTSELATILD[K([17-carboxy-heptadecanoyl]iso-Glu-
Peg3)]QAARDFIAWLYQHKITD;
H[Aib]EGSFTSELATILD[K([19-carboxy-nonadecanoyl]iso-Glu-Peg3-
Peg3)]QAARDFIAWLKQHKITD;
H[Aib]EGSFTSELATILD[K([19-carboxy-nonadecanoyl]iso-Lys-Peg3-Peg3-
Peg3)]QAARDFIAWLIQQKITD;
H[Aib]EGSFTSELATILD[K(Octadecanoyl)]QAARDFIAWLIQYKITD;
H[Aib]EGTFSSELSTILE[K(Hexadecanoyl-isoGlu)]QASREFIAWLIAYKITE;
H[Aib]EGTFSSELATILDEQAARDFIAWLIAHKITDkkkkkk([17-carboxy-Heptadecanoyl]-
isoGlu)];
H[Aib]EGTFTSELATILDEQAARDFIAWLIAHKITDkkkkkk([17-carboxy-Heptadecanoyl]-
isoGlu)];
H[Aib]EGSFTSELATILDEQAARDFIAWLIEHKITDkkkkkk([17-carboxy-Heptadecanoyl]-
isoGlu)];
H[Aib]EGTFTSELATILD[K([19-Carboxy-nonadecanoyl]-isoGlu)]QAARDFIAWLIQHKITD;
H[Aib]EGSFTSE[K([19-carboxy-nonadecanoyl]iso-Glu-Peg3-
Peg3)]ATI LDEQAARDFIAWLIEHKITD;
H[Aib]EGSFTSELATILD[K([19-carboxy-nonadecanoyl]iso-Glu-Peg3-
Peg3)]KAARDFIAWLIEHKITD;
H[Aib]EGSFTSELATILEG[K([19-carboxy-nonadecanoyl]iso-Glu-Peg3-
Peg3)]AARDFIAWLIEHKITD;
H[Aib]EGSFTSELATILDEQAA[K([19-carboxy-nonadecanoyl]iso-Glu-Peg3-
Peg3)]DFIAWLIEHKITD;
H[Aib]EGTFTSELATILDEQAA[K([19-carboxy-nonadecanoyl]iso-Glu-Peg3-
Peg3)]DFIAWLIEHKITD;
H[Aib]EGTFSSELATILD[K([17-Carboxy-heptadecanoyl]-isoGlu-KEK-
Peg3)]QAARDFIAWLIQHKITD;
H[Aib]EGTFSSELATILD[K([19-Carboxy-nonadecanoyl]-isoGlu-KEK-
Peg3)]QAARDFIAWLIQHKITD;
H[Aib]EGTFSSELATILD[K([17-Carboxy-heptadecanoyl]-isoGlu-KEK-
Peg3)]QAARDFIAWLIEHKITD;
H[Aib]EGTFSSELATILD[K([19-Carboxy-nonadecanoyl]-isoGlu-KEK-
Peg3)]QAARDFIAWLIEHKITD;
H[Aib]EGTFTSELATILD[K([19-Carboxy-nonadecanoyl]-isoGlu-
KEK)]QAARDFIAWLIQHKITD;
H[Aib]EGTFTSELATILD[K([19-Carboxy-nonadecanoyl]-isoGlu-KEK-
Peg3)]QAARDFIAWLIQHKITD;
H[Aib]EGSFTSE[K([19-Carboxy-nonadecanoyl]-isoGlu-KEK-
Peg3)]ATILDEQAARDFIAWLIEHKITD;
H[Aib]EGTFTSE[K([19-Carboxy-nonadecanoyl]-isoGlu-KEK-
Peg3)]ATILDEQAARDFIAWLIEHKITD;
H[Aib]EGSFTSE[K([19-carboxy-nonadecanoyl]iso-Glu-KEK-Peg3-
Peg3)]ATILDEQAARDFIAWLIEHKITD;
H[Aib]EGTFTSELATILD[K([19-Carboxy-nonadecanoyl]-isoGlu-KEK-
Peg3)]QAARDFIAWLIEHKITD;
H[Aib]EGSFTSELATILD[K([19-Carboxy-nonadecanoyl]-isoGlu-KEK-
Peg3)]QAARDFIAWLIEHKITD;
H[Aib]EGSFTSELATILD[K([19-Carboxy-nonadecanoyl]-isoGlu-KEK-
Peg3)]QAARDFIAWLIAHKITD;
H[Aib]EGSFTSELATILD[K([19-Carboxy-nonadecanoyl]-isoGlu-KEK-
Peg3)]KAARDFIAWLIEHKITD;
H[Aib]EGSFTSELATILD[K([19-carboxy-nonadecanoyl]iso-Glu-KEK-Peg3-
Peg3)]QAARDFIAWLIEHKITD;
H[Aib]EGSFTSELATILEG[K([19-Carboxy-nonadecanoyl]-isoGlu-KEK-
Peg3)]AARDFIAWLIEHKITD;
H[Aib]EGSFTSELATILDA[K([19-Carboxy-nonadecanoyl]-isoGlu-KEK-
Peg3)]AARDFIAWLIEHKITD;
H[Aib]EGSFTSELATILDA[K([19-carboxy-nonadecanoyl]iso-Glu-KEK-Peg3-
Peg3)]AARDFIAWLIEHKITD;
H[Aib]EGSFTSELATILDEQAA[K([19-Carboxy-nonadecanoyl]-isoGlu-KEK-
Peg3)]DFIAWLIEHKITD;
H[Aib]EGTFTSELATILDEQAA[K([19-Carboxy-nonadecanoyl]-isoGlu-KEK-
Peg3)]DFIAWLIEHKITD;
H[Aib]EGSFTSELATILDEQAA[K([19-carboxy-nonadecanoyl]iso-Glu-KEK-Peg3-
Peg3)]DFIAWLIEHKITD;
H[Aib]EGTFTSELATILDEQAA[K([19-carboxy-nonadecanoyl]iso-Glu-KEK-Peg3-
Peg3)]DFIAWLIEHKITD;
or
H[Aib]EGSFTSELATILDAKAA[K([19-Carboxy-nonadecanoyl]-isoGlu-KEK-
Peg3)]DFIAWLIEHKITD.

The dual agonist may be:

Hy-H[Aib]EGTFSSELATILD[K([17-carboxy-heptadecanoyl]-
isoGlu)]EAARDFIAWLIEHKITD-OH (Compound 1);
Hy-H[Aib]EGSFTSELATILD[K([17-carboxy-heptadecanoyl]-
isoGlu)]EAARDFIAWLIEHKITD-OH (Compound 2);
Hy-H[Aib]EGTFTSELATILD[K([17-carboxy-heptadecanoyl]-
isoGlu)]EAARDFIAWLIEHKITD-OH (Compound 3);
Hy-H[Aib]EGTFSSELATILD[K([17-carboxy-heptadecanoyl]-
isoGlu)]KAARDFIAWLIEHKITD-OH (Compound 4);
Hy-H[Aib]EGSFTSELATILD[K([17-carboxy-heptadecanoyl]-
isoGlu)]KAARDFIAWLIEHKITD-OH (Compound 5);
Hy-H[Aib]EGTFTSELATILD[K([17-carboxy-heptadecanoyl]-
isoGlu)]KAARDFIAWLIEHKITD-OH (Compound 6);
Hy-H[Aib]EGTFSSELATILDG[K([17-carboxy-heptadecanoyl]-
isoGlu)]AARDFIAWLIEHKITD-OH (Compound 7);
Hy-H[Aib]EGSFTSELATILDG[K([17-carboxy-heptadecanoyl]-
isoGlu)]AARDFIAWLIEHKITD-OH (Compound 8);
Hy-H[Aib]EGTFTSELATILDG[K([17-carboxy-heptadecanoyl]-
isoGlu)]AARDFIAWLIEHKITD-OH (Compound 9);
Hy-H[Aib]EGTFSSELATILD[K([17-carboxy-heptadecanoyl]-
isoGlu)]LAARDFIAWLIEHKITD-OH (Compound 10);
Hy-H[Aib]EGSFTSELATILD[K([17-carboxy-heptadecanoyl]-
isoGlu)]LAARDFIAWLIEHKITD-OH (Compound 11);
Hy-H[Aib]EGTFTSELATILD[K([17-carboxy-heptadecanoyl]-
isoGlu)]LAARDFIAWLIEHKITD-OH (Compound 12);
Hy-H[Aib]EGTFSSELATILD[K([17-carboxy-heptadecanoyl]-
isoGlu)]LAARDFIAWLIAHKITD-OH (Compound 13);
Hy-H[Aib]EGSFTSELATILD[K([17-carboxy-heptadecanoyl]-
isoGlu)]LAARDFIAWLIAHKITD-OH (Compound 14);
Hy-H[Aib]EGTFTSELATILD[K([17-carboxy-heptadecanoyl]-
isoGlu)]LAARDFIAWLIAHKITD-OH (Compound 15);
Hy-H[Aib]EGTFTSELATILD[K([17-carboxy-heptadecanoyl]-
isoGlu)]EAARLFIAWLIEHKITD-OH (Compound 16);
Hy-H[Aib]EGTFSSELATILD[K([17-carboxy-heptadecanoyl]-
isoGlu)]QAARDFIAWLIQHKITD-OH (Compound 17);
Hy-H[Aib]EGSFTSELATILD[K([17-carboxy-heptadecanoyl]-
isoGlu)]QAARDFIAWLIQHKITD-OH (Compound 18);
Hy-H[Aib]EGTFTSELATILD[K([17-carboxy-heptadecanoyl]-
isoGlu)]QAARDFIAWLIQHKITD-OH (Compound 19);
Hy-H[Aib]EGTFSSELATILD[K([17-carboxy-heptadecanoyl]-
isoGlu)]QAARDFIAWLIEHKITD-OH (Compound 20);
Hy-H[Aib]EGTFSSELATILD[K([17-carboxy-heptadecanoyl]-
isoGlu)]QAARDFIAWLIAHKITD-OH (Compound 21);
Hy-H[Aib]EGSFTSELATILD[K([17-carboxy-heptadecanoyl]-
isoGlu)]QAARDFIAWLIAHKITD-OH (Compound 22);
Hy-H[Aib]EGTFTSELATILD[K([17-carboxy-heptadecanoyl]-
isoGlu)]QAARDFIAWLIAHKITD-OH (Compound 23);
Hy-H[Aib]EGSFTSELATILD[K([17-carboxy-heptadecanoyl]-
isoGlu)]QAARDFIAWLIEHKITD-OH (Compound 24);
Hy-H[Aib]EGTFTSELATILD[K([17-carboxy-heptadecanoyl]-
isoGlu)]QAARDFIAWLIEHKITD-OH (Compound 25);
Hy-H[Aib]EGSFTSELATILD[K([17-carboxy-heptadecanoyl]-
isoGlu)]QAARDFIAWLIHHKITD-OH (Compound 26);
Hy-H[Aib]EGSFTSELATILD[K([17-carboxy-heptadecanoyl]-
isoGlu)]QAARDFIAWLIYHKITD-OH (Compound 27);
Hy-H[Aib]EGSFTSELATILD[K([17-carboxy-heptadecanoyl]-
isoGlu)]QAARDFIAWLILHKITD-OH (Compound 28);
Hy-H[Aib]EGSFTSELATILD[K([17-carboxy-heptadecanoyl]-
isoGlu)]QAARDFIAWLIKHKITD-OH (Compound 29);
Hy-H[Aib]EGSFTSELATILD[K([17-carboxy-heptadecanoyl]-
isoGlu)]QAARDFIAWLIRHKITD-OH (Compound 30);
Hy-H[Aib]EGSFTSELATILD[K([17-carboxy-heptadecanoyl]-
isoGlu)]QAARDFIAWLISHKITD-OH (Compound 31).
Hy-H[Aib]EGSFTSELATILD[K([Hexadecanoyl]-pAla)]QAARDFIAWLQQHKITD-OH
(Compound 32);
Hy-H[Aib]EGSFTSELATILD[K([17-carboxy-heptadecanoyl]iso-Glu-
Peg3)]QAARDFIAWLYQHKITD-OH (Compound 33);
Hy-H[Aib]EGSFTSELATILD[K([19-carboxy-nonadecanoyl]iso-Glu-Peg3-
Peg3)]QAARDFIAWLKQHKITD-OH (Compound 34);
Hy-H[Aib]EGSFTSELATILD[K([19-carboxy-nonadecanoyl]iso-Lys-Peg3-Peg3-
Peg3)]QAARDFIAWLIQQKITD-OH (Compound 35);
Hy-H[Aib]EGSFTSELATILD[K(Octadecanoyl)]QAARDFIAWLIQYKITD-OH
(Compound 36);
Hy-H[Aib]EGTFSSELSTILE[K(Hexadecanoyl-isoGlu)]QASREFIAWLIAYKITE-OH
(Compound 37);
Hy-H[Aib]EGTFSSELATILDEQAARDFIAWLIAHKITDkkkkkk([17-carboxy-
Heptadecanoyl]-isoGlu)]-[NH2] (Compound 38);
Hy-H[Aib]EGTFTSELATILDEQAARDFIAWLIAHKITDkkkkkk([17-carboxy-
Heptadecanoyl]-isoGlu)]-[NH2] (Compound 39);
Hy-H[Aib]EGSFTSELATILDEQAARDFIAWLIEHKITDkkkkkk([17-carboxy-
Heptadecanoyl]-isoGlu)]-[NH2] (Compound 40);
Hy-H[Aib]EGTFTSELATILD[K([19-Carboxy-nonadecanoyl]-
isoGlu)]QAARDFIAWLIQHKITD-OH (Compound 41);
Hy-H[Aib]EGSFTSE[K([19-carboxy-nonadecanoyl]iso-Glu-Peg3-
Peg3)]ATILDEQAARDFIAWLIEHKITD-OH (Compound 42);
Hy-H[Aib]EGSFTSELATILD[K([19-carboxy-nonadecanoyl]iso-Glu-Peg3-
Peg3)]KAARDFIAWLIEHKITD-OH (Compound 43);
Hy-H[Aib]EGSFTSELATILEG[K([19-carboxy-nonadecanoyl]iso-Glu-Peg3-
Peg3)]AARDFIAWLIEHKITD-OH (Compound 44);
Hy-H[Aib]EGSFTSELATILDEQAA[K([19-carboxy-nonadecanoyl]iso-Glu-Peg3-
Peg3)]DFIAWLIEHKITD-OH (Compound 45);
Hy-H[Aib]EGTFTSELATILDEQAA[K([19-carboxy-nonadecanoyl]iso-Glu-Peg3-
Peg3)]DFIAWLIEHKITD-OH (Compound 46).
Hy-H[Aib]EGTFSSELATILD[K([17-Carboxy-heptadecanoyl]-isoGlu-KEK-
Peg3)]QAARDFIAWLIQHKITD-OH (Compound 47);
Hy-H[Aib]EGTFSSELATILD[K([19-Carboxy-nonadecanoyl]-isoGlu-KEK-
Peg3)]QAARDFIAWLIQHKITD-OH (Compound 48);
Hy-H[Aib]EGTFSSELATILD[K([17-Carboxy-heptadecanoyl]-isoGlu-KEK-
Peg3)]QAARDFIAWLIEHKITD-OH (Compound 49);
Hy-H[Aib]EGTFSSELATILD[K([19-Carboxy-nonadecanoyl]-isoGlu-KEK-
Peg3)]QAARDFIAWLIEHKITD-OH (Compound 50);
Hy-H[Aib]EGTFTSELATILD[K([19-Carboxy-nonadecanoyl]-isoGlu-
KEK)]QAARDFIAWLIQHKITD-OH (Compound 51);
Hy-H[Aib]EGTFTSELATILD[K([19-Carboxy-nonadecanoyl]-isoGlu-KEK-
Peg3)]QAARDFIAWLIQHKITD-OH (Compound 52);
Hy-H[Aib]EGSFTSE[K([19-Carboxy-nonadecanoyl]-isoGlu-KEK-
Peg3)]ATILDEQAARDFIAWLIEHKITD-OH (Compound 53);
Hy-H[Aib]EGTFTSE[K([19-Carboxy-nonadecanoyl]-isoGlu-KEK-
Peg3)]ATILDEQAARDFIAWLIEHKITD-OH (Compound 54);
Hy-H[Aib]EGSFTSE[K([19-carboxy-nonadecanoyl]iso-Glu-KEK-Peg3-
Peg3)]ATILDEQAARDFIAWLIEHKITD-OH (Compound 55);
Hy-H[Aib]EGTFTSELATILD[K([19-Carboxy-nonadecanoyl]-isoGlu-KEK-
Peg3)]QAARDFIAWLIEHKITD-OH (Compound 56);
Hy-H[Aib]EGSFTSELATILD[K([19-Carboxy-nonadecanoyl]-isoGlu-KEK-
Peg3)]QAARDFIAWLIEHKITD-OH (Compound 57);
Hy-H[Aib]EGSFTSELATILD[K([19-Carboxy-nonadecanoyl]-isoGlu-KEK-
Peg3)]QAARDFIAWLIAHKITD-OH (Compound 58);
Hy-H[Aib]EGSFTSELATILD[K([19-Carboxy-nonadecanoyl]-isoGlu-KEK-
Peg3)]KAARDFIAWLIEHKITD-OH (Compound 59);
Hy-H[Aib]EGSFTSELATILD[K([19-carboxy-nonadecanoyl]iso-Glu-KEK-Peg3-
Peg3)]QAARDFIAWLIEHKITD-OH (Compound 60);
Hy-H[Aib]EGSFTSELATILEG[K([19-Carboxy-nonadecanoyl]-isoGlu-KEK-
Peg3)]AARDFIAWLIEHKITD-OH (Compound 61);
Hy-H[Aib]EGSFTSELATILDA[K([19-Carboxy-nonadecanoyl]-isoGlu-KEK-
Peg3)]AARDFIAWLIEHKITD-OH (Compound 62);
Hy-H[Aib]EGSFTSELATILDA[K([19-carboxy-nonadecanoyl]iso-Glu-KEK-Peg3-
Peg3)]AARDFIAWLIEHKITD-OH (Compound 63);
Hy-H[Aib]EGSFTSELATILDEQAA[K([19-Carboxy-nonadecanoyl]-isoGlu-KEK-
Peg3)]DFIAWLIEHKITD-OH (Compound 64);
Hy-H[Aib]EGTFTSELATILDEQAA[K([19-Carboxy-nonadecanoyl]-isoGlu-KEK-
Peg3)]DFIAWLIEHKITD-OH (Compound 65);
Hy-H[Aib]EGSFTSELATILDEQAA[K([19-carboxy-nonadecanoyl]iso-Glu-KEK-Peg3-
Peg3)]DFIAWLIEHKITD-OH (Compound 66);
Hy-H[Aib]EGTFTSELATILDEQAA[K([19-carboxy-nonadecanoyl]iso-Glu-KEK-Peg3-
Peg3)]DFIAWLIEHKITD-OH (Compound 67);
or
Hy-H[Aib]EGSFTSELATILDAKAA[K([19-Carboxy-nonadecanoyl]-isoGlu-KEK-
Peg3)]DFIAWLIEHKITD-OH (Compound 68).

In a preferred embodiment the dual agonist is Hy-H[Aib]EGSFTSELATILD[K([17-carboxy-heptadecanoyl]-isoGlu)]QAARDFIAWLIQHKITD-OH (Compound 18).

In an alternative aspect said GLP-1/GLP-2 dual agonist is a compound represented by the formula:

R1-X*-U-R2

wherein:

R1 is hydrogen (Hy), C1-4 alkyl (e.g. methyl), acetyl, formyl, benzoyl or trifluoroacetyl;

R2 is NH2 or OH;

X* is a peptide of formula I:

H-X2-EG-X5-F-X7-X8-E-X10-X11-TIL-X15-X16-X17-A-X19-X20-X21-FI-X24-WL-X27-X28-X29-KIT-X33 (I)

wherein

X2 is Aib or G;

X5 is S or T;

X7 is S or T;

X8 is S, E or D;

X10 is L, M or V;

X11 is A, N or S;

X15 is D or E

X16 is E, A or G;

X17 is Q, E, L or K;

X19 is A, V or S;

X20 is R or K;

X21 is D, L or E;

X24 is A, Nor S;

X27 is I, Y, Q, H or K;

X28 is A, E, H, Y, L, K, Q, R or S;

X29 is H, Y, K or Q;

X33 is D or E;

U is absent or a sequence of 1-15 residues, each independently selected from K and k; and wherein at least one of X5 and X7 is T;

or a pharmaceutically acceptable salt or solvate thereof.

The dual agonist according to the invention may be in the form of a pharmaceutically acceptable salt or solvate, such as a pharmaceutically acceptable acid addition salt.

The composition according to the invention may comprise the dual agonist, or a pharmaceutically acceptable salt or solvate thereof, together with a carrier, excipient or vehicle. The carrier may be a pharmaceutically acceptable carrier.

The composition may be a pharmaceutical composition. The pharmaceutical composition may be formulated as a liquid suitable for administration by injection or infusion. It may be formulated to achieve slow release of the dual agonist.

The composition may be administered at a dose of about 0.1 pmol/kg to 500 μmol/kg body weight.

In one aspect the composition may be effective to increase intestinal mass and/or increase the villus/crypt ratio in the intestinal mucosa.

A further aspect provides a therapeutic kit comprising a composition comprising a dual agonist according to the invention for use in the treatment of a patient who is receiving parenteral nutrition.

### FIGURE LEGENDS

FIG. 1: Mass of proximal small intestine for piglets receiving enteral nutrition (EN), total parenteral nutrition (TPN) with vehicle or total parenteral nutrition (TPN) with one of four different dose levels of compound 18 (0.008 mg/kg, 0.033 mg/kg, 0.066 mg/kg, 0.133 mg/kg).

FIG. 2: Mass of distal small intestine for piglets receiving enteral nutrition (EN), total parenteral nutrition (TPN) with vehicle and total parenteral nutrition (TPN) with one of four different dose levels of compound 18 (0.008 mg/kg, 0.033 mg/kg, 0.066 mg/kg, 0.133 mg/kg).

FIG. 3: Villus/crypt ratio for piglets receiving enteral nutrition (EN), total parenteral nutrition (TPN)+vehicle and total parenteral nutrition (TPN)+compound 18 in a dose of 0.133 mg/kg.

FIG. 4: Histology of villus from piglet receiving enteral nutrition.

FIG. 5: Histology of villus from piglet receiving total parenteral nutrition (TPN) with vehicle.

FIG. 6: Histology of villus from piglet receiving total parenteral nutrition (TPN) with compound 18 at a dose of 0.133 mg/kg.

### DETAILED DESCRIPTION OF THE INVENTION

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well known and commonly used in the art.

All patents, published patent applications and non-patent publications referred to in this application are specifically incorporated by reference herein. In case of conflict, the present specification, including its specific definitions, will control.

Each embodiment of the invention described herein may be taken alone or in combination with one or more other embodiments of the invention.

Definitions

Unless specified otherwise, the following definitions are provided for specific terms which are used in the present written description.

Throughout this specification, the word “comprise”, and grammatical variants thereof, such as “comprises” or “comprising”, will be understood to imply the inclusion of a stated integer or component, or group of integers or components, but not the exclusion of any other integer or component, or group of integers or components.

The singular forms “a,” “an,” and “the” include the plurals unless the context clearly dictates otherwise.

The term “including” is used to mean “including but not limited to”. “Including” and “including but not limited to” may be used interchangeably.

The terms “patient”, “subject” and “individual” may be used interchangeably and refer to either a human or a non-human animal. These terms include mammals such as humans, primates, livestock animals (e.g., bovines and porcines), companion animals (e.g., canines and felines) and rodents (e.g., mice and rats). In one aspect the patient is a human.

The term “solvate” in the context of the present invention refers to a complex of defined stoichiometry formed between a solute (in casu, a peptide or pharmaceutically acceptable salt thereof according to the invention) and a solvent. The solvent in this connection may, for example, be water, ethanol or another pharmaceutically acceptable, typically small-molecular organic species, such as, but not limited to, acetic acid or lactic acid. When the solvent in question is water, such a solvate is normally referred to as a hydrate.

The term “agonist” as employed in the context of the invention refers to a substance (ligand) that activates the receptor type in question.

Throughout the present description and claims the conventional three-letter and one-letter codes for naturally occurring amino acids are used, i.e.

A (Ala), G (Gly), L (Leu), I (Ile), V (Val), F (Phe), W (Trp), S (Ser), T (Thr), Y (Tyr), N (Asn), Q (Gin), D (Asp), E (Glu), K (Lys), R (Arg), H (His), M (Met), C (Cys) and P (Pro);

as well as generally accepted three-letter codes for other a-amino acids, such as sarcosine (Sar), norleucine (Nle), a-aminoisobutyric acid (Aib), 2,3-diaminopropanoic acid (Dap), 2,4-diaminobutanoic acid (Dab) and 2,5-diaminopentanoic acid (ornithine; Orn). Such other α-amino acids may be shown in square brackets “[ ]” (e.g. “[Aib]”) when used in a general formula or sequence in the present specification, especially when the rest of the formula or sequence is shown using the single letter code. Unless otherwise specified, amino acid residues in peptides of the invention are of the L-configuration. However, D-configuration amino acids may be incorporated. In the present context, an amino acid code written with a small letter represents the D-configuration of said amino acid, e.g. “k” represents the D-configuration of lysine (K).

Among sequences disclosed herein are sequences incorporating a “Hy-”moiety at the amino terminus (N-terminus) of the sequence, and either an “—OH” moiety or an “—NH₂” moiety at the carboxy terminus (C-terminus) of the sequence. In such cases, and unless otherwise indicated, a “Hy-” moiety at the N-terminus of the sequence in question indicates a hydrogen atom [i.e. R¹=hydrogen=Hy in the general formulas; corresponding to the presence of a free primary or secondary amino group at the N-terminus], while an “—OH” or an “—NH₂” moiety at the C-terminus of the sequence indicates a hydroxy group [e.g. R²=OH in general formulas; corresponding to the presence of a carboxy (COOH) group at the C-terminus] or an amino group [e.g. R²=[NH₂] in the general formulas; corresponding to the presence of an amido (CONH₂) group at the C-terminus], respectively. In each sequence of the invention, a C-terminal “—OH” moiety may be substituted for a C-terminal “—NH₂” moiety, and vice-versa.

“Percent (%) amino acid sequence identity” with respect to the GLP-2 polypeptide sequences is defined as the percentage of amino acid residues in a candidate sequence that are identical to the amino acid residues in the wild-type (human) GLP-2 sequence, after aligning the sequences and introducing gaps, if necessary, to achieve the maximum percent sequence identity, and not considering any conservative substitutions as part of the sequence identity. Sequence alignment can be carried out by the skilled person using techniques well known in the art, for example using publicly available software such as BLAST, BLAST2 or Align software. For examples, see Altschul et al., Methods in Enzymology 266: 460-480 (1996) or Pearson et al., Genomics 46: 24-36, 1997.

The percentage sequence identities used herein in the context of the present invention may be determined using these programs with their default settings. More generally, the skilled worker can readily determine appropriate parameters for determining alignment, including any algorithms needed to achieve maximal alignment over the full length of the sequences being compared.

Dual Agonist Compounds

The terms “GLP-1/GLP-2 dual agonist” and “GLP-1/GLP-2 dual receptor agonist” are used interchangeably herein and have the same meaning. The dual agonists/dual receptor agonists have agonist activity at both of the GLP-1 and GLP-2 receptors, e.g. the human GLP-1 and GLP-2 receptors.

In accordance with the present invention, the dual agonist has at least one GLP-1 and at least one GLP-2 biological activity.

Exemplary GLP-1 physiological activities include reducing rate of intestinal transit, reducing rate of gastric emptying, reducing appetite, food intake or body weight, and improving glucose control and glucose tolerance. Exemplary GLP-2 physiological activities include causing an increase in intestinal mass (e.g. of small intestine or colon), intestinal repair, and improving intestinal barrier function (i.e. reducing permeability of the intestine). These parameters can be assessed in in vivo assays in which the mass and the permeability of the intestine, or a portion thereof, is determined after a test animal has been treated with a dual agonist.

The dual agonists have agonist activity at the GLP-1 and GLP-2 receptors, e.g. the human GLP-1 and GLP-2 receptors. EC₅₀ values for in vitro receptor agonist activity may be used as a numerical measure of agonist potency at a given receptor. An EC₅₀ value is a measure of the concentration (e.g. mol/L) of a compound required to achieve half of that compound's maximal activity in a particular assay. A compound having a numerical EC₅₀ at a particular receptor which is lower than the EC₅₀ of a reference compound in the same assay may be considered to have higher potency at that receptor than the reference compound.

GLP-1 Activity

In some embodiments, the dual agonist has an EC₅₀ at the GLP-1 receptor (e.g. the human GLP-1 receptor) which is below 2.0 nM, below 1.5 nM, below 1.0 nM, below 0.9 nM, below 0.8 nM, below 0.7 nM, below 0.6 nM, below 0.5 nM, below 0.4 nM, below 0.3 nM, below 0.2 nM, below 0.1 nM, below 0.09 nM, below 0.08 nM, below 0.07 nM, below 0.06 nM, below 0.05 nM, below 0.04 nM, e.g. when assessed using the GLP-1 receptor potency assay described in Example 2 of WO2018/104561.

In some embodiments, the dual agonist has an EC₅₀ at the GLP-1 receptor which is between 0.005 and 2.5 nM, between 0.01 nM and 2.5 nM, between 0.025 and 2.5 nM, between 0.005 and 2.0 nM, between 0.01 nM and 2.0 nM, between 0.025 and 2.0 nM, between 0.005 and 1.5 nM, between 0.01 nM and 1.5 nM, between 0.025 and 1.5 nM, between 0.005 and 1.0 nM, between 0.01 nM and 1.0 nM, between 0.025 and 1.0 nM, between 0.005 and 0.5 nM, between 0.01 nM and 0.5 nM, between 0.025 and 0.5 nM, between 0.005 and 0.25 nM, between 0.01 nM and 0.25 nM, between 0.025 and 0.25 nM, e.g. when assessed using the GLP-1 receptor potency assay described in Example 2 of WO2018/104561.

An alternative measure of GLP-1 agonist activity may be derived by comparing the potency of a dual agonist with the potency of a known (or reference) GLP-1 agonist when both are measured in the same assay. Thus the relative potency at the GLP-1 receptor may be defined as:

[EC₅₀(reference agonist)]/[EC₅₀(dual agonist)].

Thus a value of 1 indicates that the dual agonist and reference agonist have equal potency, a value of >1 indicates that the dual agonist has higher potency (i.e. lower EC₅₀) than the reference agonist, and a value of <1 indicates that the dual agonist has lower potency (i.e. higher EC₅₀) than the reference agonist.

The reference GLP-1 agonist may, for example, be human GLP-1(7-37), liraglutide (NN2211; Victoza), or Exendin-4, but is preferably liraglutide.

Typically the relative potency will be between 0.001 and 100, e.g.

between 0.001 and 10, between 0.001 and 5, between 0.001 and 1, between 0.001 and 0.5, between 0.001 and 0.1, between 0.001 and 0.05, or between 0.001 and 0.01;

between 0.01 and 10, between 0.01 and 5, between 0.01 and 1, between 0.01 and 0.5, between 0.01 and 0.1, or between 0.01 and 0.05;

between 0.05 and 10, between 0.05 and 5, between 0.05 and 1, between 0.05 and 0.5, or between 0.05 and 0.1;

between 0.1 and 10, between 0.1 and 5, between 0.1 and 1, or between 0.1 and 0.5;

between 0.5 and 10, between 0.5 and 5, or between 0.5 and 1;

between 1 and 10, or between 1 and 5;

or between 5 and 10.

The dual agonist according to the invention may have higher potency at the GLP-1 receptor (e.g. the human GLP-1 receptor) than wild type human GLP-2 (hGLP-2 (1-33)) or [Gly2]-hGLP-2 (1-33) (i.e. human GLP-2 having glycine at position 2, also known as teduglutide). Thus, the relative potency of the dual agonists at the GLP-1 receptor compared to hGLP-2 (1-33) or teduglutide is greater than 1, typically greater than 5 or greater than 10, and may be up to 100, up to 500, or even higher.

GLP-2 Activity

In some embodiments, the dual agonist has an EC₅₀ at the GLP-2 receptor (e.g. the human GLP-2 receptor) which is below 2.0 nM, below 1.5 nM, below 1.0 nM, below 0.9 nM, below 0.8 nM, below 0.7 nM, below 0.6 nM, below 0.5 nM, below 0.4 nM, below 0.3 nM, below 0.2 nM, below 0.1 nM, below 0.09 nM, below 0.08 nM, below 0.07 nM, below 0.06 nM, below 0.05 nM, below 0.04 nM, below 0.03 nM, below 0.02 nM, or below 0.01 nM, e.g. when assessed using the GLP-1 receptor potency assay described in Example 2 of WO2018/104561.

In some embodiments, the dual agonist has an EC₅₀ at the GLP-2 receptor which is between 0.005 and 2.0 nM, between 0.01 nM and 2.0 nM, between 0.025 and 2.0 nM, between 0.005 and 1.5 nM, between 0.01 nM and 1.5 nM, between 0.025 and 1.5 nM, between 0.005 and 1.0 nM, between 0.01 nM and 1.0 nM, between 0.025 and 1.0 nM, between 0.005 and 0.5 nM, between 0.01 nM and 0.5 nM, between 0.025 and 0.5 nM, between 0.005 and 0.25 nM, between 0.01 nM and 0.25 nM, between 0.025 and 0.25 nM, e.g. when assessed using the GLP-2 receptor potency assay described in Example 2 of WO2018/104561.

An alternative measure of GLP-2 agonist activity may be derived by comparing the potency of a dual agonist with the potency of a known (or reference) GLP-2 agonist when both are measured in the same assay. Thus the relative potency at the GLP-2 receptor may be defined as:

[EC₅₀(reference agonist)]/[EC₅₀(dual agonist)].

Thus a value of 1 indicates that the dual agonist and reference agonist have equal potency, a value of >1 indicates that the dual agonist has higher potency (i.e. lower EC₅₀) than the reference agonist, and a value of <1 indicates that the dual agonist has lower potency (i.e. higher EC₅₀) than the reference agonist.

The reference GLP-2 agonist may, for example, be human GLP-2(1-33) or teduglutide ([Gly2]-hGLP-2 (1-33)), but is preferably teduglutide. Typically the relative potency will be between 0.001 and 100, e.g.

between 0.001 and 10, between 0.001 and 5, between 0.001 and 1, between 0.001 and 0.5, between 0.001 and 0.1, between 0.001 and 0.05, or between 0.001 and 0.01;

between 0.01 and 10, between 0.01 and 5, between 0.01 and 1, between 0.01 and 0.5, between 0.01 and 0.1, or between 0.01 and 0.05;

between 0.05 and 10, between 0.05 and 5, between 0.05 and 1, between 0.05 and 0.5, or between 0.05 and 0.1;

between 0.1 and 10, between 0.1 and 5, between 0.1 and 1, or between 0.1 and 0.5;

between 0.5 and 10, between 0.5 and 5, or between 0.5 and 1;

between 1 and 10, or between 1 and 5;

or between 5 and 10.

The dual agonists according to the invention may have higher potency at the GLP-2 receptor (e.g. the human GLP-2 receptor) than human GLP-1(7-37), liraglutide (NN2211; Victoza), or Exendin-4. Thus, the relative potency of the dual agonists at the GLP-2 receptor compared to human GLP-1(7-37), liraglutide (NN2211; Victoza), or Exendin-4 is greater than 1, typically greater than 5 or greater than 10, and may be up to 100, up to 500, or even higher (if the reference GLP-1 agonist even exerts detectable activity at the GLP-2 receptor).

It will be understood that the absolute potencies of the dual agonists at each receptor are much less important than the balance between the GLP-1 and GLP-2 agonist activities. Thus it is perfectly acceptable for the absolute GLP-1 or GLP-2 potency to be lower than that of known agonists at those receptors, as long as the dual agonist compound exerts acceptable relative levels of potency at both receptors. Any apparent deficiency in absolute potency can be compensated by an increased dose if required.

Substituents

The dual agonist may contain a residue ψ which comprises a residue of Lys, Arg, Orn, Dap or Dab in which the side chain is conjugated to a substituent Z¹— or Z¹—Z²— wherein Z¹ represents a moiety CH3—(CH₂)₁₀₋₂₂—(CO)— or HOOC—(CH₂)₁₀₋₂₂—(CO)— and Z² when present represents a spacer.

The spacer Z² is selected from —Z^{S1}—, —Z^{S1}—Z^{S2}—, —Z^{S2}—Z^{S1}—, —Z^{S2}—, —Z^{S3}—, —Z^{S1}Z^{S3}—, —Z^{S2}Z^{S3}—, —Z^{S3}Z^{S1}—, —Z^{S3}Z^{S2}—, —Z^{S1}Z^{S2}Z^{S3}—, —Z^{S1}Z^{S3}Z^{S2}—, —Z^{S2}Z^{S1}Z^{S3}—, —Z^{S2}Z^{S3}Z^{S1}—, —Z^{S3}Z^{S1}Z^{S2}—, —Z^{S3}Z^{S2}Z^{S1}—, Z^{S2}Z^{S3}Z^{S2}— wherein

Z^{S1} is isoGlu, β-Ala, isoLys, or 4-aminobutanoyl;

Z^{S2} is -(Peg3)ₘ- where m is 1, 2, or 3; and

Z^{S3}— is a peptide sequence of 1-6 amino acid units selected from the group consisting of A, L, S, T, Y, Q, D, E, K, k, R, H, F and G.

In some embodiments, Z² is a spacer of the formula —Z^{S1}—Z^{S2}—, —Z^{S2}—Z^{S1}, or Z^{S2}, where —Z^{S1}— is isoGlu, β-Ala, isoLys, or 4-aminobutanoyl; and —Z^{S2}— is -(Peg3)ₘ- where m is 1, 2, or 3.

Without wishing to be bound by theory, it is believed that the hydrocarbon chain of Z¹ binds albumin in the blood stream, thus shielding the dual agonists of the present invention from enzymatic degradation, which can enhance the half-life of the dual agonists.

The substituent may also modulate the potency of the dual agonists, with respect to the GLP-2 receptor and/or the GLP-1 receptor.

The substituent Z¹— or Z¹—Z²— is conjugated to the functional group at the distal end of the side-chain from the alpha-carbon of the relevant amino acid residue. The normal ability of the amino acid (Lys, Arg, Orn, Dab, Dap) side-chain in question to participate in interactions mediated by that functional group (e.g. intra- and inter-molecular interactions) may therefore be reduced or completely eliminated by the presence of the substituent. Thus, the overall properties of the dual agonist may be relatively insensitive to changes in the actual amino acid conjugated to the substituent. Consequently, it is believed that any of the residues Lys, Arg, Orn, Dab, or Dap may be present at any position where ψ is permitted. However, in certain embodiments, it may be advantageous that the amino acid to which the substituent is conjugated is Lys or Orn.

The moiety Z¹ may be covalently bonded to the functional group in the amino acid side-chain, or alternatively may be conjugated to the amino acid side-chain functional group via a spacer Z².

The term “conjugated” is used here to describe the covalent attachment of one identifiable chemical moiety to another, and the structural relationship between such moieties. It should not be taken to imply any particular method of synthesis.

The bonds between Z¹, Z^{S1}, Z^{S2}; Z^{S3} and the amino acid side chain to which the substituent is bound (collectively referred to herein as ψ) are peptidic. In other words, the units may be joined by amide condensation reactions.

Z¹ comprises a hydrocarbon chain having from 10 to 24 carbon (C) atoms, such as from 10 to 22 C atoms, e.g. from 10 to 20 C atoms. Preferably, it has at least 10 or at least 11 C atoms, and preferably it has 20 C atoms or fewer, e.g. 18 C atoms or fewer. For example, the hydrocarbon chain may contain 12, 13, 14, 15, 16, 17, 18, 19 or 20 carbon atoms. For example, it may contain 18 or 20 carbon atoms.

In some embodiments, Z¹ is a group selected from dodecanoyl, tetradecanoyl, hexadecanoyl, octadecanoyl and eicosanoyl, preferably hexadecanoyl, octadecanoyl or eicosanoyl, more preferably octadecanoyl or eicosanoyl.

Alternative Z¹ groups are derived from long-chain saturated α, ω-dicarboxylic acids of formula HOOC—(CH₂)₁₂₋₂₂—COOH, preferably from long-chain saturated α, ω-dicarboxylic acids having an even number of carbon atoms in the aliphatic chain. For example, Z¹ may be:

13-carboxytridecanoyl, i.e. HOOC—(CH₂)₁₂—(CO)—;

15-carboxypentadecanoyl, i.e. HOOC—(CH₂)₁₄—(CO)—;

17-carboxyheptadecanoyl, i.e. HOOC—(CH₂)₁₆—(CO)—;

19-carboxynonadecanoyl, i.e. HOOC—(CH₂)₁₈—(CO)—; or

21-carboxyheneicosanoyl, i.e. HOOC—(CH₂)₂₀—(CO)—.

As mentioned above, Z¹ may be conjugated to the amino acid side-chain by a spacer Z². When present, the spacer is attached to Z¹ and to the amino acid side-chain.

The spacer Z₂ has the —Z^{S1}—, —Z^{S1}—Z^{S2}—, —Z^{S2}—Z^{S1}—, —Z^{S2}—, —Z^{S3}—, —Z^{S1}Z^{S3}—, —Z^{S2}Z^{S3}—, —Z^{S3}Z^{S1}—, —Z^{S3}Z^{S2}—, —Z^{S1}Z^{S2}Z^{S3}—, —Z^{S1}Z^{S3}Z^{S2}—, —Z^{S2}Z^{S1}Z^{S3}—, —Z^{S2}Z^{S3}Z^{S1}—, —Z^{S3}Z^{S1}Z^{S2}—, —Z^{S3}Z^{S2}Z^{S1}—, Z^{S2}Z^{S3}Z^{S2}—;

where

—Z^{S1} is isoGlu, β-Ala, isoLys, or 4-aminobutanoyl;

—Z^{S2}— is -(Peg3)ₘ- where m is 1, 2, or 3; and

—Z^{S3}— is a peptide sequence of 1-6 amino acid units independently selected from the group consisting of A (Ala), L (Leu), S (Ser), T (Thr), Y (Tyr), Q (Gin), D (Asp), E (Glu), K (L-Lys), k (D-Lys), R (Arg), H (His), F (Phe) and G (Gly).

The terms “isoGlu” and “isoLys” indicate residues of amino acids which participate in bonds via their side chain carboxyl or amine functional groups. Thus isoGlu participates in bonds via its alpha amino and side chain carboxyl group, while isoLys participates via its carboxyl and side chain amino groups. In the context of the present specification, the terms “y-Glu” and “isoGlu” are used interchangeably.

The term Peg3 is used to refer to an 8-amino-3,6-dioxaoctanoyl group.

Z^{S3} may, for example, be 3 to 6 amino acids in length, i.e. 3, 4, 5 or 6 amino acids in length.

In some embodiments, the amino acids of Z^{S3} are independently selected from K, k, E, A, T, I and L, e.g. from K, k, E and A, e.g. from K, k and E.

Typically Z^{S3} includes at least one charged amino acid (K, k, R or E, e.g. K, k or E) and preferably two or more charged amino acids. In some embodiments it includes at least 2 positively charged amino acids (K, k or R, especially K or k), or at least 1 positively charged amino acid (K, k or R, especially K or k) and at least one negatively charged amino acid (E). In some embodiments, all amino acid residues of Z^{S3} are charged. For example, Z^{S3} may be a chain of alternately positively and negatively charged amino acids.

Examples of Z^{S3} moieties include KEK, EKEKEK, kkkkkk, EkEkEk, AKAAEK, AKEKEK and ATILEK.

Without being bound by theory, it is believed that the incorporation of Z^{S3} into the linker between the fatty acid chain and the peptide backbone may increase the half-life of the dual agonist by enhancing its affinity for serum albumin.

In some embodiments, —Z²— is -Z^{S1}— or —Z^{S1}—Z^{S2}—; in other words, —Z²— is selected from:

isoGlu(Peg3)₀₋₃;

β-Ala(Peg3)₀₋₃;

isoLys(Peg3)₀₋₃; and

4-aminobutanoyl(Peg3)₀₋₃.

Thus, certain examples of substituents Z¹— include

[Dodecanoyl], [Tetradecanoyl], [Hexadecanoyl], [Octadecanoyl], [Eicosanoyl],

[13-Carboxy-tridecanoyl], [15-Carboxy-pentadecanoyl], [17-Carboxy-heptadecanoyl], [19-Carboxy-nonadecanoyl], [21-carboxy-heneicosanoyl].

More broadly, —Z²— may be —Z^{S1}—, —Z^{S2}—, —Z^{S3}—Z^{S1}—, —Z^{S1}—Z^{S3}—, —Z^{S1}—Z^{S3}—Z^{S2}—, —Z^{S3}—Z^{S2}—Z^{S1}— or Z^{S3}—. Thus, —Z²— may be selected from the group consisting of:

isoGlu(Peg3)₀₋₃;

β-Ala(Peg3)₀₋₃;

isoLys(Peg3)₀₋₃;

4-aminobutanoyl(Peg3)₀₋₃;

isoGlu(KEK)(Peg3)₀₋₃;

β-Ala(KEK)(Peg3)₀₋₃;

isoLys(KEK)(Peg3)₀₋₃;

4-aminobutanoyl(KEK)(Peg3)₀₋₃;

KEK(isoGlu);

KEK(β-Ala);

KEK(isoLys);

KEK(4-aminobutanoyl);

isoGlu(KEK);

β-Ala(KEK);

isoLys(KEK);

4-aminobutanoyl(KEK);

KEK(isoGlu)(Peg3)₀₋₃;

KEK(β-Ala)(Prg3)₀₋₃;

KEK(isoLys)(Peg3)₀₋₃; and

KEK(4-aminobutanoyl)(Peg3)₀₋₃;

Certain examples of substituents Z¹—Z²— include:

[Dodecanoyl]-isoGlu, [Tetradecanoyl]-isoGlu, [Hexadecanoyl]-isoGlu, [Octadecanoyl]-isoGlu, [Eicosanoyl]-isoGlu,

[Hexadecanoyl]-βAla, [Octadecanoyl]-βAla, [Eicosanoyl]-βAla, [Tetradecanoyl]-βAla, [Dodecanoyl]-βAla,

[Dodecanoyl]-isoGlu-Peg3, [Tetradecanoyl]-isoGlu-Peg3, [Hexadecanoyl]-isoGlu-Peg3, [Octadecanoyl]-isoGlu-Peg3, [Eicosanoyl]-isoGlu-Peg3,

[Dodecanoyl]-βAla-Peg3, [Tetradecanoyl]-βAla-Peg3, [Hexadecanoyl]-βAla-Peg3, [Octadecanoyl]-βAla-Peg3, [Eicosanoyl]-βAla-Peg3,

[Dodecanoyl]-isoGlu-Peg3-Peg3, [Tetradecanoyl]-isoGlu-Peg3-Peg3, [Hexadecanoyl]-isoGlu-Peg3-Peg3, [Octadecanoyl]-isoGlu-Peg3-Peg3, [Eicosanoyl]-isoGlu-Peg3-Peg3,

[Dodecanoyl]-βAla-Peg3-Peg3, [Tetradecanoyl]-βAla-Peg3-Peg3, [Hexadecanoyl]-βAla-Peg3-Peg3, [Octadecanoyl]-βAla-Peg3-Peg3, [Eicosanoyl]-βAla-Peg3-Peg3,

[Dodecanoyl]-isoGlu-Peg3-Peg3-Peg3, [Tetradecanoyl]-isoGlu-Peg3-Peg3-Peg3, [Hexadecanoyl]-isoGlu-Peg3-Peg3-Peg3, [Octadecanoyl]-isoGlu-Peg3-Peg3-Peg3, [Eicosanoyl]-isoGlu-Peg3-Peg3-Peg3,

[Dodecanoyl]-βAla-Peg3-Peg3-Peg3, [Tetradecanoyl]-βAla-Peg3-Peg3-Peg3, [Hexadecanoyl]-βAla-Peg3-Peg3-Peg3, [Octadecanoyl]-βAla-Peg3-Peg3-Peg3,

[Eicosanoyl]-βAla-Peg3-Peg3-Peg3,

[Dodecanoyl]-isoLys, [Tetradecanoyl]-isoLys, [Hexadecanoyl]-isoLys, [Octadecanoyl]-isoLys, [Eicosanoyl]-isoLys,

[Hexadecanoyl]-[4-aminobutanoyl], [Octadecanoyl]-[4-aminobutanoyl], [Eicosanoyl]-[4-aminobutanoyl], [Tetradecanoyl]-[4-aminobutanoyl], [Dodecanoyl]-[4-aminobutanoyl],

[Dodecanoyl]-isoLys-Peg3, [Tetradecanoyl]-isoLys-Peg3, [Hexadecanoyl]-isoLys-Peg3, [Octadecanoyl]-isoLys-Peg3, [Eicosanoyl]-isoLys-Peg3,

[Dodecanoyl]-[4-aminobutanoyl]-Peg3, [Tetradecanoyl]-[4-aminobutanoyl]-Peg3, [Hexadecanoyl]-[4-aminobutanoyl]-Peg3,

[Octadecanoyl]-[4-aminobutanoyl]-Peg3, [Eicosanoyl]-[4-aminobutanoyl]Peg3,

[Dodecanoyl]-isoLys-Peg3-Peg3, [Tetradecanoyl]-isoLys-Peg3-Peg3, [Hexadecanoyl]-isoLys-Peg3-Peg3, [Octadecanoyl]-isoLys-Peg3-Peg3, [Eicosanoyl]-isoLys-Peg3-Peg3,

[Dodecanoyl]-[4-aminobutanoyl]-Peg3-Peg3, [Tetradecanoyl]-[4-aminobutanoyl]-Peg3-Peg3, [Hexadecanoyl]-[4-aminobutanoyl]Peg3-Peg3, [Octadecanoyl]-[4-aminobutanoyl]-Peg3-Peg3, [Eicosanoyl]-[4-aminobutanoyl]-Peg3-Peg3,

[Dodecanoyl]-isoLys-Peg3-Peg3-Peg3, [Tetradecanoyl]-isoLys-Peg3-Peg3-Peg3, [Hexadecanoyl]-isoLys-Peg3-Peg3-Peg3, [Octadecanoyl]-isoLys-Peg3-Peg3-Peg3, [Eicosanoyl]-isoLys-Peg3-Peg3-Peg3,

[Dodecanoyl]-[4-aminobutanoyl]-Peg3-Peg3-Peg3, [Tetradecanoyl]-[4-aminobutanoyl]-Peg3-Peg3-Peg3, [Hexadecanoyl]-[4-aminobutanoyl]-Peg3-Peg3-Peg3, [Octadecanoyl]-[4-aminobutanoyl]-Peg3-Peg3-Peg3, [Eicosanoyl]-[4-aminobutanoyl]-Peg3-Peg3-Peg3,

[13-carboxy-tridecanoyl]-isoGlu, [15-carboxy-Pentadecanoyl]-isoGlu, [17-carboxy-Heptadecanoyl]-isoGlu, [19-carboxy-Nonadecanoyl]-isoGlu, [21-carboxy-heneicosanoyl]-isoGlu,

[17-carboxy-Heptadecanoyl]-βAla, [19-carboxy-Nonadecanoyl]-βAla, [21-carboxy-heneicosanoyl]-βAla, [15-carboxy-Pentadecanoyl]-βAla, [13-carboxy-tridecanoyl]-βAla,

[13-carboxy-tridecanoyl]-isoGlu-Peg3, [15-carboxy-Pentadecanoyl]-isoGlu-Peg3, [17-carboxy-Heptadecanoyl]-isoGlu-Peg3, [19-carboxy-Nonadecanoyl]-isoGlu-Peg3, [21-carboxy-heneicosanoyl]-isoGlu-Peg3,

[13-carboxy-tridecanoyl]-βAla-Peg3, [15-carboxy-Pentadecanoyl]-βAla-Peg3, [17-carboxy-Heptadecanoyl]-βAla-Peg3, [19-carboxy-Nonadecanoyl]-βAla-Peg3, [21-carboxy-heneicosanoyl]-βAla-Peg3,

[13-carboxy-tridecanoyl]-isoGlu-Peg3-Peg3, [15-carboxy-Pentadecanoyl]-isoGlu-Peg3-Peg3, [17-carboxy-Heptadecanoyl]-isoGlu-Peg3-Peg3, [19-carboxy-Nonadecanoyl]-isoGlu-Peg3-Peg3, [21-carboxy-heneicosanoyl]-isoGlu-Peg3-Peg3,

[13-carboxy-tridecanoyl]-βAla-Peg3-Peg3, [15-carboxy-Pentadecanoyl]-βAla-Peg3-Peg3, [17-carboxy-Heptadecanoyl]-βAla-Peg3-Peg3, [19-carboxy-Nonadecanoyl]-βAla-Peg3-Peg3, [21-carboxy-heneicosanoyl]-βAla-Peg3-Peg3,

[13-carboxy-tridecanoyl]-isoGlu-Peg3-Peg3-Peg3, [15-carboxy-Pentadecanoyl]-isoGlu-Peg3-Peg3-Peg3, [17-carboxy-Heptadecanoyl]-isoGlu-Peg3-Peg3-Peg3, [19-carboxy-Nonadecanoyl]-isoGlu-Peg3-Peg3-Peg3, [21-carboxy-heneicosanoyl]-isoGlu-Peg3-Peg3-Peg3,

[13-carboxy-tridecanoyl]-βAla-Peg3-Peg3-Peg3, [15-carboxy-Pentadecanoyl]-βAla-Peg3-Peg3-Peg3, [17-carboxy-Heptadecanoyl]-βAla-Peg3-Peg3-Peg3, [19-carboxy-Nonadecanoyl]-βAla-Peg3-Peg3-Peg3, [21-carboxy-heneicosanoyl]-βAla-Peg3-Peg3-Peg3,

[13-carboxy-tridecanoyl]-isoLys, [15-carboxy-Pentadecanoyl]-isoLys, [17-carboxy-Heptadecanoyl]-isoLys, [19-carboxy-Nonadecanoyl]-isoLys, [21-carboxy-heneicosanoyl]-isoLys,

[17-carboxy-Heptadecanoyl]-[4-aminobutanoyl], [19-carboxy-Nonadecanoyl]-[4-aminobutanoyl], [21-carboxy-heneicosanoyl]-[4-aminobutanoyl], [15-carboxy-Pentadecanoyl]-[4-aminobutanoyl], [13-carboxy-tridecanoyl]-[4-aminobutanoyl],

[13-carboxy-tridecanoyl]-isoLys-Peg3, [15-carboxy-Pentadecanoyl]-isoLys-Peg3, [17-carboxy-Heptadecanoyl]-isoLys-Peg3, [19-carboxy-Nonadecanoyl]-isoLys-Peg3, [21-carboxy-heneicosanoyl]-isoLys-Peg3,

[13-carboxy-tridecanoyl]-[4-aminobutanoyl]-Peg3, [15-carboxy-Pentadecanoyl]-[4-aminobutanoyl]-Peg3, [17-carboxy-Heptadecanoyl]-[4-aminobutanoyl]-Peg3,

[19-carboxy-Nonadecanoyl]-βAla-Peg3, [21-carboxy-heneicosanoyl]-βAla-Peg3,

[13-carboxy-tridecanoyl]-isoLys-Peg3-Peg3, [15-carboxy-Pentadecanoyl]-isoLys-Peg3-Peg3, [17-carboxy-Heptadecanoyl]-isoLys-Peg3-Peg3, [19-carboxy-Nonadecanoyl]-isoLys-Peg3-Peg3, [21-carboxy-heneicosanoyl]-isoLys-Peg3-Peg3,

[13-carboxy-tridecanoyl]-[4-aminobutanoyl]-Peg3-Peg3, [15-carboxy-Pentadecanoyl]-[4-aminobutanoyl]-Peg3-Peg3, [17-carboxy-Heptadecanoyl]-[4-aminobutanoyl]-Peg3-Peg3,

[19-carboxy-Nonadecanoyl]-[4-aminobutanoyl]-Peg3-Peg3, [21-carboxy-heneicosanoyl]-[4-aminobutanoyl]-Peg3-Peg3,

[13-carboxy-tridecanoyl]-isoLys-Peg3-Peg3-Peg3, [15-carboxy-Pentadecanoyl]-isoLys-Peg3-Peg3-Peg3, [17-carboxy-Heptadecanoyl]-isoLys-Peg3-Peg3-Peg3, [19-carboxy-Nonadecanoyl]-isoLys-Peg3-Peg3-Peg3, [21-carboxy-heneicosanoyl]-isoLys-Peg3-Peg3-Peg3,

[13-carboxy-tridecanoyl]-[4-aminobutanoyl]-Peg3-Peg3-Peg3, [15-carboxy-Pentadecanoyl]-[4-aminobutanoyl]-Peg3-Peg3-Peg3, [17-carboxy-Heptadecanoyl]-[4-aminobutanoyl]-Peg3-Peg3-Peg3, [19-carboxy-Nonadecanoyl]-[4-aminobutanoyl]-Peg3-Peg3-Peg3 and [21-carboxy-heneicosanoyl]-[4-aminobutanoyl]-Peg3-Peg3-Peg3.

Further examples of substituents Z¹-Z²— include:

[Dodecanoyl]-isoLys, [Tetradecanoyl]-isoLys, [Hexadecanoyl]-isoLys, [Octadecanoyl]-isoLys, [Eicosanoyl]-isoLys,

[Hexadecanoyl]-[4-aminobutanoyl], [Octadecanoyl]-[4-aminobutanoyl], [Eicosanoyl]-[4-aminobutanoyl], [Tetradecanoyl]-[4-aminobutanoyl], [Dodecanoyl]-[4-aminobutanoyl],

[Hexadecanoyl]-KEK, [Octadecanoyl]-KEK, [Eicosanoyl]-KEK, [Tetradecanoyl]-KEK, [Dodecanoyl]-KEK,

[Dodecanoyl]-Peg3, [Tetradecanoyl]-Peg3, [Hexadecanoyl]-Peg3, [Octadecanoyl]-Peg3, [Eicosanoyl]-Peg3,

[Dodecanoyl]-Peg3-Peg3, [Tetradecanoyl]-Peg3-Peg3, [Hexadecanoyl]-Peg3-Peg3, [Octadecanoyl]-Peg3-Peg3, [Eicosanoyl]-Peg3-Peg3,

[Dodecanoyl]-Peg3-Peg3-Peg3, [Tetradecanoyl]-Peg3-Peg3-Peg3, [Hexadecanoyl]-Peg3-Peg3-Peg3, [Octadecanoyl]-Peg3-Peg3-Peg3, [Eicosanoyl]-Peg3-Peg3-Peg3,

[Dodecanoyl]-isoLys-Peg3, [Tetradecanoyl]-isoLys-Peg3, [Hexadecanoyl]-isoLys-Peg3, [Octadecanoyl]-isoLys-Peg3, [Eicosanoyl]-isoLys-Peg3,

[Dodecanoyl]-[4-aminobutanoyl]-Peg3, [Tetradecanoyl]-[4-aminobutanoyl]-Peg3, [Hexadecanoyl]-[4-aminobutanoyl]-Peg3, [Octadecanoyl]-[4-aminobutanoyl]-Peg3, [Eicosanoyl]-[4-aminobutanoyl]-Peg3,

[Dodecanoyl]-KEK-Peg3, [Tetradecanoyl]-KEK-Peg3, [Hexadecanoyl]-KEK-Peg3, [Octadecanoyl]-KEK-Peg3, [Eicosanoyl]-KEK-Peg3,

[Dodecanoyl]-isoLys-Peg3-Peg3, [Tetradecanoyl]-isoLys-Peg3-Peg3, [Hexadecanoyl]-isoLys-Peg3-Peg3, [Octadecanoyl]-isoLys-Peg3-Peg3, [Eicosanoyl]-isoLys-Peg3-Peg3,

[Dodecanoyl]-[4-aminobutanoyl]-Peg3-Peg3, [Tetradecanoyl]-[4-aminobutanoyl]-Peg3-Peg3, [Hexadecanoyl]-[4-aminobutanoyl]-Peg3-Peg3, [Octadecanoyl]-[4-aminobutanoyl]-Peg3-Peg3, [Eicosanoyl]-[4-aminobutanoyl]-Peg3-Peg3,

[Dodecanoyl]-KEK-Peg3-Peg3, [Tetradecanoyl]-KEK-Peg3-Peg3, [Hexadecanoyl]-KEK-Peg3-Peg3, [Octadecanoyl]-KEK -Peg3-Peg3, [Eicosanoyl]-KEK -Peg3-Peg3,

[Dodecanoyl]-isoLys-Peg3-Peg3-Peg3, [Tetradecanoyl]-isoLys-Peg3-Peg3-Peg3, [Hexadecanoyl]-isoLys-Peg3-Peg3-Peg3, [Octadecanoyl]-isoLys-Peg3-Peg3-Peg3, [Eicosanoyl]-isoLys-Peg3-Peg3-Peg3,

[Dodecanoyl]-[4-aminobutanoyl]-Peg3-Peg3-Peg3, [Tetradecanoyl]-[4-aminobutanoyl]-Peg3-Peg3-Peg3, [Hexadecanoyl]-[4-aminobutanoyl]-Peg3-Peg3-Peg3, [Octadecanoyl]-[4-aminobutanoyl]-Peg3-Peg3-Peg3, [Eicosanoyl]-[4-aminobutanoyl]-Peg3-Peg3-Peg3,

[Dodecanoyl]-KEK-Peg3-Peg3-Peg3, [Tetradecanoyl]-KEK-Peg3-Peg3-Peg3, [Hexadecanoyl]-KEK-Peg3-Peg3-Peg3, [Octadecanoyl]-KEK-Peg3-Peg3-Peg3, [Eicosanoyl]-KEK-Peg3-Peg3-Peg3,

[Dodecanoyl]-isoGlu-KEK-Peg3, [Tetradecanoyl]-isoGlu-KEK-Peg3, [Hexadecanoyl]-isoGlu-KEK-Peg3, [Octadecanoyl]-isoGlu-KEK-Peg3, [Eicosanoyl]-isoGlu-KEK-Peg3,

[Dodecanoyl]-[4-aminobutanoyl]-KEK-Peg3, [Tetradecanoyl]-[4-aminobutanoyl]-KEK-Peg3, [Hexadecanoyl]-[4-aminobutanoyl]-KEK-Peg3, [Octadecanoyl]-[4-aminobutanoyl]-KEK-Peg3, [Eicosanoyl]-[4-aminobutanoyl]-KEK-Peg3,

[Dodecanoyl]-isoLys-KEK-Peg3, [Tetradecanoyl]-isoLys-KEK-Peg3, [Hexadecanoyl]-isoLys-KEK-Peg3, [Octadecanoyl]-isoLys-KEK-Peg3, [Eicosanoyl]-isoLys-KEK-Peg3,

[Dodecanoyl]-βAla-KEK-Peg3, [Tetradecanoyl]-βAla-KEK-Peg3, [Hexadecanoyl]-βAla-KEK-Peg3, [Octadecanoyl]-βAla-KEK-Peg3, [Eicosanoyl]-βAla-KEK-Peg3,

[Dodecanoyl]-isoGlu-KEK-Peg3-Peg3, [Tetradecanoyl]-isoGlu-KEK-Peg3-Peg3, [Hexadecanoyl]-isoGlu-KEK-Peg3-Peg3, [Octadecanoyl]-isoGlu-KEK-Peg3-Peg3, [Eicosanoyl]-isoGlu-KEK-Peg3-Peg3,

[Dodecanoyl]-βAla-KEK-Peg3-Peg3, [Tetradecanoyl]-βAla-KEK-Peg3-Peg3, [Hexadecanoyl]-βAla-KEK-Peg3-Peg3, [Octadecanoyl]-βAla-KEK-Peg3-Peg3, [Eicosanoyl]-βAla-KEK-Peg3-Peg3,

[Dodecanoyl]-isoLys-KEK-Peg3-Peg3, [Tetradecanoyl]-isoLys-KEK-Peg3-Peg3, [Hexadecanoyl]-isoLys-KEK-Peg3-Peg3, [Octadecanoyl]-isoLys-KEK-Peg3-Peg3, [Eicosanoyl]-isoLys-KEK-Peg3-Peg3,

[Dodecanoyl]-[4-aminobutanoyl]-KEK-Peg3-Peg3, [Tetradecanoyl]-[4-aminobutanoyl]-KEK-Peg3-Peg3, [Hexadecanoyl]-[4-aminobutanoyl]-KEK-Peg3-Peg3, [Octadecanoyl]-[4-aminobutanoyl]-KEK-Peg3-Peg3, [Eicosanoyl]-[4-aminobutanoyl]-KEK-Peg3-Peg3,

[Dodecanoyl]-isoGlu-KEK-Peg3-Peg3-Peg3, [Tetradecanoyl]-isoGlu-KEK-Peg3-Peg3-Peg3, [Hexadecanoyl]-isoGlu-KEK-Peg3-Peg3-Peg3, [Octadecanoyl]-isoGlu-KEK-Peg3-Peg3-Peg3, [Eicosanoyl]-isoGlu-KEK-Peg3-Peg3-Peg3,

[Dodecanoyl]-βAla-KEK-Peg3-Peg3-Peg3, [Tetradecanoyl]-βAla-KEK-Peg3-Peg3-Peg3, [Hexadecanoyl]-βAla-KEK-Peg3-Peg3-Peg3, [Octadecanoyl]-βAla-KEK-Peg3-Peg3-Peg3, [Eicosanoyl]-βAla-KEK-Peg3-Peg3-Peg3,

[Dodecanoyl]-isoLys-KEK-Peg3-Peg3-Peg3, [Tetradecanoyl]-isoLys-KEK-Peg3-Peg3-Peg3, [Hexadecanoyl]-isoLys-KEK-Peg3-Peg3-Peg3, [Octadecanoyl]-isoLys-KEK-Peg3-Peg3-Peg3, [Eicosanoyl]-isoLys-KEK-Peg3-Peg3-Peg3,

[Dodecanoyl]-[4-aminobutanoyl]-KEK-Peg3-Peg3-Peg3, [Tetradecanoyl]-[4-aminobutanoyl]-KEK-Peg3-Peg3-Peg3, [Hexadecanoyl]-[4-aminobutanoyl]-KEK-Peg3-Peg3-Peg3, [Octadecanoyl]-[4-aminobutanoyl]-KEK-Peg3-Peg3-Peg3, [Eicosanoyl]-[4-aminobutanoyl]-KEK-Peg3-Peg3-Peg3,

[Dodecanoyl]-KEK-isoGlu-Peg3, [Tetradecanoyl]-KEK-isoGlu-Peg3, [Hexadecanoyl]-KEK-isoGlu-Peg3, [Octadecanoyl]-KEK-isoGlu-Peg3, [Eicosanoyl]-KEK-isoGlu-Peg3,

[Dodecanoyl]-KEK-βAla-Peg3, [Tetradecanoyl]-KEK-βAla-Peg3, [Hexadecanoyl]-KEK-βAla-Peg3, [Octadecanoyl]-KEK-βAla-Peg3, [Eicosanoyl]-KEK-βAla-Peg3,

[Dodecanoyl]-KEK-[4-aminobutanoyl]-Peg3, [Tetradecanoyl]-KEK-[4-aminobutanoyl]-Peg3, [Hexadecanoyl]-KEK-[4-aminobutanoyl]-Peg3, [Octadecanoyl]-KEK-[4-aminobutanoyl]-Peg3, [Eicosanoyl]-KEK-[4-aminobutanoyl]-Peg3,

[Dodecanoyl]-KEK-isoLys-Peg3, [Tetradecanoyl]-KEK-isoLys-Peg3, [Hexadecanoyl]-KEK-isoLys-Peg3, [Octadecanoyl]-KEK-isoLys-Peg3, [Eicosanoyl]-KEK-isoLys-Peg3,

[Dodecanoyl]-KEK-isoGlu-Peg3-Peg3, [Tetradecanoyl]-KEK-isoGlu-Peg3-Peg3, [Hexadecanoyl]-KEK-isoGlu-Peg3-Peg3, [Octadecanoyl]-KEK-isoGlu-Peg3-Peg3, [Eicosanoyl]-KEK-isoGlu-Peg3-Peg3,

[Dodecanoyl]-KEK-βAla-Peg3-Peg3, [Tetradecanoyl]-KEK-βAla-Peg3-Peg3, [Hexadecanoyl]-KEK-βAla-Peg3-Peg3, [Octadecanoyl]-KEK-βAla-Peg3-Peg3, [Eicosanoyl]-βAla-KEK-Peg3-Peg3,

[Dodecanoyl]-KEK-[4-aminobutanoyl]-Peg3-Peg3, [Tetradecanoyl]-KEK-[4-aminobutanoyl]-Peg3-Peg3, [Hexadecanoyl]-KEK-[4-aminobutanoyl]-Peg3-Peg3, [Octadecanoyl]-KEK-[4-aminobutanoyl]-Peg3-Peg3, [Eicosanoyl]-KEK-[4-aminobutanoyl]-Peg3-Peg3,

[Dodecanoyl]-KEK-isoLys-Peg3-Peg3, [Tetradecanoyl]-KEK-isoLys-Peg3-Peg3, [Hexadecanoyl]-KEK-isoLys-Peg3-Peg3, [Octadecanoyl]-KEK-isoLys-Peg3-Peg3, [Eicosanoyl]-KEK-isoLys-Peg3-Peg3,

[Dodecanoyl]-KEK-isoGlu-Peg3-Peg3-Peg3, [Tetradecanoyl]-KEK-isoGlu-Peg3-Peg3-Peg3, [Hexadecanoyl]-KEK-isoGlu-Peg3-Peg3-Peg3, [Octadecanoyl]-KEK-isoGlu-Peg3-Peg3-Peg3, [Eicosanoyl]-KEK-isoGlu-Peg3-Peg3-Peg3,

[Dodecanoyl]-KEK-βAla-Peg3-Peg3-Peg3, [Tetradecanoyl]KEK-βAla-Peg3-Peg3-Peg3, [Hexadecanoyl]-βAla-KEK-Peg3-Peg3-Peg3, [Octadecanoyl]-KEK-βAla-Peg3-Peg3-Peg3, [Eicosanoyl]-KEK-βAla-Peg3-Peg3-Peg3,

[Dodecanoyl]-KEK-[4-aminobutanoyl]-Peg3-Peg3-Peg3, [Tetradecanoyl]-KEK-[4-aminobutanoyl]-Peg3-Peg3-Peg3, [Hexadecanoyl]-KEK-[4-aminobutanoyl]-Peg3-Peg3-Peg3, [Octadecanoyl]-KEK-[4-aminobutanoyl]-Peg3-Peg3-Peg3, [Eicosanoyl]-KEK-[4-aminobutanoyl]-Peg3-Peg3-Peg3,

[Dodecanoyl]-KEK-isoLys-Peg3-Peg3-Peg3, [Tetradecanoyl]-KEK-isoLys-Peg3-Peg3-Peg3, [Hexadecanoyl]-KEK-isoLys-Peg3-Peg3-Peg3, [Octadecanoyl]-KEK-isoLys-Peg3-Peg3-Peg3, [Eicosanoyl]-KEK-isoLys-Peg3-Peg3-Peg3,

[13-carboxy-tridecanoyl]-isoGlu, [15-carboxy-Pentadecanoyl]-isoGlu, [17-carboxy-Heptadecanoyl]-isoGlu, [19-carboxy-Nonadecanoyl]-isoGlu, [21-carboxy-hen21-carboxy-heneicosanoyl]-isoGlu,

[17-carboxy-Heptadecanoyl]-βAla, [19-carboxy-Nonadecanoyl]-βAla, [21-carboxy-heneicosanoyl]-βAla, [15-carboxy-Pentadecanoyl]-βAla, [13-carboxy-tridecanoyl]-βAla,

[13-carboxy-tridecanoyl]-isoLys, [15-carboxy-Pentadecanoyl]-isoLys, [17-carboxy-Heptadecanoyl]-isoLys, [19-carboxy-Nonadecanoyl]-isoLys, [21-carboxy-heneicosanoyl]-isoLys,

[17-carboxy-Heptadecanoyl]-[4-aminobutanoyl], [19-carboxy-Nonadecanoyl]-[4-aminobutanoyl], [21-carboxy-heneicosanoyl]-[4-aminobutanoyl], [15-carboxy-Pentadecanoyl]-[4-aminobutanoyl], [13-carboxy-tridecanoyl]-[4-aminobutanoyl],

[17-carboxy-Heptadecanoyl]-KEK, [19-carboxy-Nonadecanoyl]-KEK, [21-carboxy-heneicosanoyl]-KEK, [15-carboxy-Pentadecanoyl]-KEK, [13-carboxy-tridecanoyl]-KEK,

[13-carboxy-tridecanoyl]-Peg3, [15-carboxy-Pentadecanoyl]-Peg3, [17-carboxy-Heptadecanoyl]-Peg3, [19-carboxy-Nonadecanoyl]-Peg3, [21-carboxy-heneicosanoyl]-Peg3,

[13-carboxy-tridecanoyl]-Peg3-Peg3, [15-carboxy-Pentadecanoyl]-Peg3-Peg3,

[17-carboxy-Heptadecanoyl]-Peg3-Peg3, [19-carboxy-Nonadecanoyl]-Peg3-Peg3, [21-carboxy-heneicosanoyl]-Peg3-Peg3,

[13-carboxy-tridecanoyl]-Peg3-Peg3-Peg3, [15-carboxy-Pentadecanoyl]-Peg3-Peg3-Peg3, [17-carboxy-Heptadecanoyl]-Peg3-Peg3-Peg3, [19-carboxy-Nonadecanoyl]-Peg3-Peg3-Peg3, [21-carboxy-heneicosanoyl]-Peg3-Peg3-Peg3,

[13-carboxy-tridecanoyl]-isoGlu-Peg3, [15-carboxy-Pentadecanoyl]-isoGlu-Peg3, [17-carboxy-Heptadecanoyl]-isoGlu-Peg3, [19-carboxy-Nonadecanoyl]-isoGlu-Peg3, [21-carboxy-heneicosanoyl]-isoGlu-Peg3,

[13-carboxy-tridecanoyl]-βAla-Peg3, [15-carboxy-Pentadecanoyl]-βAla-Peg3, [17-carboxy-Heptadecanoyl]-βAla-Peg3, [19-carboxy-Nonadecanoyl]-βAla-Peg3, [21-carboxy-heneicosanoyl]-βAla-Peg3,

[13-carboxy-tridecanoyl]-isoLys-Peg3, [15-carboxy-Pentadecanoyl]-isoLys-Peg3, [17-carboxy-Heptadecanoyl]-isoLys-Peg3, [19-carboxy-Nonadecanoyl]-isoLys-Peg3, [21-carboxy-heneicosanoyl]-isoLys-Peg3,

[13-carboxy-tridecanoyl]-[4-aminobutanoyl]-Peg3, [15-carboxy-Pentadecanoyl]-[4-aminobutanoyl]-Peg3, [17-carboxy-Heptadecanoyl]-[4-aminobutanoyl]-Peg3, [19-carboxy-Nonadecanoyl]-[4-aminobutanoyl]-Peg3, [21-carboxy-heneicosanoyl]-[4-aminobutanoyl]-Peg3,

[13-carboxy-tridecanoyl]-KEK-Peg3, [15-carboxy-Pentadecanoyl]-KEK-Peg3, [17-carboxy-Heptadecanoyl]-KEK-Peg3, [19-carboxy-Nonadecanoyl]-KEK-Peg3, [21-carboxy-heneicosanoyl]-KEK-Peg3,

[13-carboxy-tridecanoyl]-isoGlu-Peg3-Peg3, [15-carboxy-Pentadecanoyl]-isoGlu-Peg3-Peg3, [17-carboxy-Heptadecanoyl]-isoGlu-Peg3-Peg3, [19-carboxy-Nonadecanoyl]-isoGlu-Peg3-Peg3, [21-carboxy-heneicosanoyl]-isoGlu-Peg3-Peg3,

[13-carboxy-tridecanoyl]-βAla-Peg3-Peg3, [15-carboxy-Pentadecanoyl]-βAla-Peg3-Peg3, [17-carboxy-Heptadecanoyl]-βAla-Peg3-Peg3, [19-carboxy-Nonadecanoyl]-βAla-Peg3-Peg3, [21-carboxy-heneicosanoyl]-βAla-Peg3-Peg3,

[13-carboxy-tridecanoyl]-isoLys-Peg3-Peg3, [15-carboxy-Pentadecanoyl]-isoLys-Peg3-Peg3, [17-carboxy-Heptadecanoyl]-isoLys-Peg3-Peg3, [19-carboxy-Nonadecanoyl]-isoLys-Peg3-Peg3, [21-carboxy-heneicosanoyl]-isoLys-Peg3-Peg3,

[13-carboxy-tridecanoyl]-[4-aminobutanoyl]-Peg3-Peg3, [15-carboxy-Pentadecanoyl]-[4-aminobutanoyl]-Peg3-Peg3, [17-carboxy-Heptadecanoyl]-[4-aminobutanoyl]-Peg3-Peg3, [19-carboxy-Nonadecanoyl]-[4-aminobutanoyl]-Peg3-Peg3, [21-carboxy-heneicosanoyl]-[4-aminobutanoyl]-Peg3-Peg3,

[13-carboxy-tridecanoyl]-KEK-Peg3-Peg3, [15-carboxy-Pentadecanoyl]-KEK-Peg3-Peg3, [17-carboxy-Heptadecanoyl]-KEK-Peg3-Peg3, [19-carboxy-Nonadecanoyl]-KEK -Peg3-Peg3, [21-carboxy-heneicosanoyl]-KEK -Peg3-Peg3,

[13-carboxy-tridecanoyl]-isoGlu-Peg3-Peg3-Peg3, [15-carboxy-Pentadecanoyl]-isoGlu-Peg3-Peg3-Peg3, [17-carboxy-Heptadecanoyl]-isoGlu-Peg3-Peg3-Peg3, [19-carboxy-Nonadecanoyl]-isoGlu-Peg3-Peg3-Peg3, [21-carboxy-heneicosanoyl]-isoGlu-Peg3-Peg3-Peg3,

[13-carboxy-tridecanoyl]-βAla-Peg3-Peg3-Peg3, [15-carboxy-Pentadecanoyl]-βAla-Peg3-Peg3-Peg3, [17-carboxy-Heptadecanoyl]-βAla-Peg3-Peg3-Peg3, [19-carboxy-Nonadecanoyl]-βAla-Peg3-Peg3-Peg3, [21-carboxy-heneicosanoyl]-βAla-Peg3-Peg3-Peg3,

[13-carboxy-tridecanoyl]-isoLys-Peg3-Peg3-Peg3, [15-carboxy-Pentadecanoyl]-isoLys-Peg3-Peg3-Peg3, [17-carboxy-Heptadecanoyl]-isoLys-Peg3-Peg3-Peg3, [19-carboxy-Nonadecanoyl]-isoLys-Peg3-Peg3-Peg3, [21-carboxy-heneicosanoyl]-isoLys-Peg3-Peg3-Peg3,

[13-carboxy-tridecanoyl]-[4-aminobutanoyl]-Peg3-Peg3-Peg3, [15-carboxy-Pentadecanoyl]-[4-aminobutanoyl]-Peg3-Peg3-Peg3, [17-carboxy-Heptadecanoyl]-[4-aminobutanoyl]-Peg3-Peg3-Peg3, [19-carboxy-Nonadecanoyl]-[4-aminobutanoyl]-Peg3-Peg3-Peg3, [21-carboxy-heneicosanoyl]-[4-aminobutanoyl]-Peg3-Peg3-Peg3,

[13-carboxy-tridecanoyl]-KEK-Peg3-Peg3-Peg3, [15-carboxy-Pentadecanoyl]-KEK-Peg3-Peg3-Peg3, [17-carboxy-Heptadecanoyl]-KEK-Peg3-Peg3-Peg3, [19-carboxy-Nonadecanoyl]-KEK-Peg3-Peg3-Peg3, [21-carboxy-heneicosanoyl]-KEK-Peg3-Peg3-Peg3,

[13-carboxy-tridecanoyl]-isoGlu-KEK-Peg3, [15-carboxy-Pentadecanoyl]-isoGlu-KEK-Peg3, [17-carboxy-Heptadecanoyl]-isoGlu-KEK-Peg3, [19-carboxy-Nonadecanoyl]-isoGlu-KEK-Peg3, [21-carboxy-heneicosanoyl]-isoGlu-KEK-Peg3,

[13-carboxy-tridecanoyl]-[4-aminobutanoyl]-KEK-Peg3, [15-carboxy-Pentadecanoyl]-[4-aminobutanoyl]-KEK-Peg3, [17-carboxy-Heptadecanoyl]-[4-aminobutanoyl]-KEK-Peg3, [19-carboxy-Nonadecanoyl]-[4-aminobutanoyl]-KEK-Peg3, [21-carboxy-heneicosanoyl]-[4-aminobutanoyl]-KEK-Peg3,

[13-carboxy-tridecanoyl]-isoLys-KEK-Peg3, [15-carboxy-Pentadecanoyl]-isoLys-KEK-Peg3, [17-carboxy-Heptadecanoyl]-isoLys-KEK-Peg3, [19-carboxy-Nonadecanoyl]-isoLys-KEK-Peg3, [21-carboxy-heneicosanoyl]-isoLys-KEK-Peg3,

[13-carboxy-tridecanoyl]-βAla-KEK-Peg3, [15-carboxy-Pentadecanoyl]-βAla-KEK-Peg3, [17-carboxy-Heptadecanoyl]-βAla-KEK-Peg3, [19-carboxy-Nonadecanoyl]-βAla-KEK-Peg3, [21-carboxy-heneicosanoyl]-βAla-KEK-Peg3,

[13-carboxy-tridecanoyl]-isoGlu-KEK-Peg3-Peg3, [15-carboxy-Pentadecanoyl]-isoGlu-KEK-Peg3-Peg3, [17-carboxy-Heptadecanoyl]-isoGlu-KEK-Peg3-Peg3, [19-carboxy-Nonadecanoyl]-isoGlu-KEK-Peg3-Peg3, [21-carboxy-heneicosanoyl]-isoGlu-KEK-Peg3-Peg3,

[13-carboxy-tridecanoyl]-βAla-KEK-Peg3-Peg3, [15-carboxy-Pentadecanoyl]-βAla-KEK-Peg3-Peg3, [17-carboxy-Heptadecanoyl]-βAla-KEK-Peg3-Peg3, [19-carboxy-Nonadecanoyl]-βAla-KEK-Peg3-Peg3, [21-carboxy-heneicosanoyl]-βAla-KEK-Peg3-Peg3,

[13-carboxy-tridecanoyl]-isoLys-KEK-Peg3-Peg3, [15-carboxy-Pentadecanoyl]-isoLys-KEK-Peg3-Peg3, [17-carboxy-Heptadecanoyl]-isoLys-KEK-Peg3-Peg3, [19-carboxy-Nonadecanoyl]-isoLys-KEK-Peg3-Peg3, [21-carboxy-heneicosanoyl]-isoLys-KEK-Peg3-Peg3,

[13-carboxy-tridecanoyl]-[4-aminobutanoyl]-KEK-Peg3-Peg3, [15-carboxy-Pentadecanoyl]-[4-aminobutanoyl]-KEK-Peg3-Peg3, [17-carboxy-Heptadecanoyl]-[4-aminobutanoyl]-KEK-Peg3-Peg3, [19-carboxy-Nonadecanoyl]-[4-aminobutanoyl]-KEK-Peg3-Peg3, [21-carboxy-heneicosanoyl]-[4-aminobutanoyl]-KEK-Peg3-Peg3,

[13-carboxy-tridecanoyl]-isoGlu-KEK-Peg3-Peg3-Peg3, [15-carboxy-Pentadecanoyl]-isoGlu-KEK-Peg3-Peg3-Peg3, [17-carboxy-Heptadecanoyl]-isoGlu-KEK-Peg3-Peg3-Peg3, [19-carboxy-Nonadecanoyl]-isoGlu-KEK-Peg3-Peg3-Peg3, [21-carboxy-heneicosanoyl]-isoGlu-KEK-Peg3-Peg3-Peg3,

[13-carboxy-tridecanoyl]-βAla-KEK-Peg3-Peg3-Peg3, [15-carboxy-Pentadecanoyl]-βAla-KEK-Peg3-Peg3-Peg3, [17-carboxy-Heptadecanoyl]-βAla-KEK-Peg3-Peg3-Peg3, [19-carboxy-Nonadecanoyl]-βAla-KEK-Peg3-Peg3-Peg3, [21-carboxy-heneicosanoyl]-βAla-KEK-Peg3-Peg3-Peg3,

[13-carboxy-tridecanoyl]-isoLys-KEK-Peg3-Peg3-Peg3, [15-carboxy-Pentadecanoyl]-isoLys-KEK-Peg3-Peg3-Peg3, [17-carboxy-Heptadecanoyl]-isoLys-KEK-Peg3-Peg3-Peg3, [19-carboxy-Nonadecanoyl]-isoLys-KEK-Peg3-Peg3-Peg3, [21-carboxy-heneicosanoyl]-isoLys-KEK-Peg3-Peg3-Peg3,

[13-carboxy-tridecanoyl]-[4-aminobutanoyl]-KEK-Peg3-Peg3-Peg3, [15-carboxy-Pentadecanoyl]-[4-aminobutanoyl]-KEK-Peg3-Peg3-Peg3, [17-carboxy-Heptadecanoyl]-[4-aminobutanoyl]-KEK-Peg3-Peg3-Peg3, [19-carboxy-Nonadecanoyl]-[4-aminobutanoyl]-KEK-Peg3-Peg3-Peg3, [21-carboxy-heneicosanoyl]-[4-aminobutanoyl]-KEK-Peg3-Peg3-Peg3,

[13-carboxy-tridecanoyl]-KEK-isoGlu-Peg3, [15-carboxy-Pentadecanoyl]-KEK-isoGlu-Peg3, [17-carboxy-Heptadecanoyl]-KEK-isoGlu-Peg3, [19-carboxy-Nonadecanoyl]-KEK-isoGlu-Peg3, [21-carboxy-heneicosanoyl]-KEK-isoGlu-Peg3,

[13-carboxy-tridecanoyl]-KEK-βAla-Peg3, [15-carboxy-Pentadecanoyl]-KEK-βAla-Peg3, [17-carboxy-Heptadecanoyl]-KEK-βAla-Peg3, [19-carboxy-Nonadecanoyl]-KEK-βAla-Peg3, [21-carboxy-heneicosanoyl]-KEK-βAla-Peg3,

[13-carboxy-tridecanoyl]-KEK[4-aminobutanoyl]-Peg3, [15-carboxy-Pentadecanoyl]-KEK-[4-aminobutanoyl]-Peg3, [17-carboxy-Heptadecanoyl]-KEK[4-aminobutanoyl]-Peg3, [19-carboxy-Nonadecanoyl]-KEK-[4-aminobutanoyl]-Peg3, [21-carboxy-heneicosanoyl]-KEK-[4-aminobutanoyl]-Peg3,

[13-carboxy-tridecanoyl]-KEK-isoLys-Peg3, [15-carboxy-Pentadecanoyl]-KEK-isoLys-Peg3, [17-carboxy-Heptadecanoyl]-KEK-isoLys-Peg3, [19-carboxy-Nonadecanoyl]-KEK-isoLys-Peg3, [21-carboxy-heneicosanoyl]-KEK-isoLys-Peg3,

[13-carboxy-tridecanoyl]-KEK-isoGlu-Peg3-Peg3, [15-carboxy-Pentadecanoyl]-KEK-isoGlu-Peg3-Peg3, [17-carboxy-Heptadecanoyl]-KEK-isoGlu-Peg3-Peg3, [19-carboxy-Nonadecanoyl]-KEK-isoGlu-Peg3-Peg3, [21-carboxy-heneicosanoyl]-KEK-isoGlu-Peg3-Peg3,

[13-carboxy-tridecanoyl]-KEK-βAla-Peg3-Peg3, [15-carboxy-Pentadecanoyl]-KEK-βAla-Peg3-Peg3, [17-carboxy-Heptadecanoyl]-KEK-βAla-Peg3-Peg3, [19-carboxy-Nonadecanoyl]-KEK-βAla-Peg3-Peg3, [21-carboxy-heneicosanoyl]-βAla-KEK-Peg3-Peg3,

[13-carboxy-tridecanoyl]-KEK[4-aminobutanoyl]-Peg3-Peg3, [15-carboxy-Pentadecanoyl]-KEK-[4-aminobutanoyl]-Peg3-Peg3, [17-carboxy-Heptadecanoyl]-KEK-[4-aminobutanoyl]-Peg3-Peg3, [19-carboxy-Nonadecanoyl]-KEK-[4-aminobutanoyl]-Peg3-Peg3, [21-carboxy-heneicosanoyl]-KEK-[4-aminobutanoyl]-Peg3-Peg3,

[13-carboxy-tridecanoyl]-KEK-isoLys-Peg3-Peg3, [15-carboxy-Pentadecanoyl]-KEK-isoLys-Peg3-Peg3, [17-carboxy-Heptadecanoyl]-KEK-isoLys-Peg3-Peg3, [19-carboxy-Nonadecanoyl]-KEK-isoLys-Peg3-Peg3, [21-carboxy-heneicosanoyl]-KEK-isoLys-Peg3-Peg3,

[13-carboxy-tridecanoyl]-KEK-isoGlu-Peg3-Peg3-Peg3, [15-carboxy-Pentadecanoyl]-KEK-isoGlu-Peg3-Peg3-Peg3, [17-carboxy-Heptadecanoyl]-KEK-isoGlu-Peg3-Peg3-Peg3, [19-carboxy-Nonadecanoyl]-KEK-isoGlu-Peg3-Peg3-Peg3, [21-carboxy-heneicosanoyl]-KEK-isoGlu-Peg3-Peg3-Peg3,

[13-carboxy-tridecanoyl]-KEK-βAla-Peg3-Peg3-Peg3, [15-carboxy-Pentadecanoyl]KEK-βAla-Peg3-Peg3-Peg3, [17-carboxy-Heptadecanoyl]-βAla-KEK-Peg3-Peg3-Peg3, [19-carboxy-Nonadecanoyl]-KEK-βAla-Peg3-Peg3-Peg3, [21-carboxy-heneicosanoyl]-KEK-βAla-Peg3-Peg3-Peg3,

[13-carboxy-tridecanoyl]-KEK[4-aminobutanoyl]-Peg3-Peg3-Peg3, [15-carboxy-Pentadecanoyl]-KEK-[4-aminobutanoyl]-Peg3-Peg3-Peg3, [17-carboxy-Heptadecanoyl]-KEK-[4-aminobutanoyl]-Peg3-Peg3-Peg3, [19-carboxy-Nonadecanoyl]-KEK-[4-aminobutanoyl]-Peg3-Peg3-Peg3, [21-carboxy-heneicosanoyl]-KEK-[4-aminobutanoyl]-Peg3-Peg3-Peg3,

[13-carboxy-tridecanoyl]-KEK-isoLys-Peg3-Peg3-Peg3, [15-carboxy-Pentadecanoyl]-KEK-isoLys-Peg3-Peg3-Peg3, [17-carboxy-Heptadecanoyl]-KEK-isoLys-Peg3-Peg3-Peg3, [19-carboxy-Nonadecanoyl]-KEK-isoLys-Peg3-Peg3-Peg3, [21-carboxy-heneicosanoyl]-KEK-isoLys-Peg3-Peg3-Peg3.

Certain preferred substituents Z¹— and Z¹—Z²— include:

[Hexadecanoyl], [Octadecanoyl], [17-Carboxy-heptadecanoyl], [19-Carboxy-nonadecanoyl],

[Hexadecanoyl]-isoGlu, [Octadecanoyl]-isoGlu,

[Hexadecanoyl]-βAla, [Octadecanoyl]-βAla,

[Hexadecanoyl]-isoGlu-Peg3,

[Hexadecanoyl]-βAla-Peg3,

[Hexadecanoyl]-isoGlu-Peg3-Peg3,

[Hexadecanoyl]-βAla-Peg3-Peg3,

[Hexadecanoyl]-βAla-Peg3-Peg3-Peg3,

[Hexadecanoyl]-isoLys,

[Hexadecanoyl]-[4-aminobutanoyl],

[Hexadecanoyl]-isoLys-Peg3,

[Hexadecanoyl][4-aminobutanoyl]-Peg3,

[Hexadecanoyl]-isoLys-Peg3-Peg3,

[Hexadecanoyl][4-aminobutanoyl]-Peg3-Peg3,

[Hexadecanoyl]-isoLys-Peg3-Peg3-Peg3,

[Hexadecanoyl][4-aminobutanoyl]-Peg3-Peg3-Peg3,

[17-carboxy-Heptadecanoyl]-isoGlu,

[19-carboxy-Nonadecanoyl]-isoGlu,

[17-carboxy-Heptadecanoyl]-βAla,

[19-carboxy-Nonadecanoyl]-βAla,

[17-carboxy-Heptadecanoyl]-isoGlu-Peg3,

[19-carboxy-Nonadecanoyl]-isoGlu-Peg3,

[17-carboxy-Heptadecanoyl]-βAla-Peg3,

[19-carboxy-Nonadecanoyl]-βAla-Peg3,

[17-carboxy-Heptadecanoyl]-isoGlu-Peg3-Peg3,

[19-carboxy-Nonadecanoyl]-isoGlu-Peg3-Peg3,

[17-carboxy-Heptadecanoyl]-βAla-Peg3-Peg3,

[19-carboxy-Nonadecanoyl]-βAla-Peg3-Peg3,

[17-carboxy-Heptadecanoyl]-isoGlu-Peg3-Peg3-Peg3,

[19-carboxy-Nonadecanoyl]-isoGlu-Peg3-Peg3-Peg3,

[17-carboxy-Heptadecanoyl]-βAla-Peg3-Peg3-Peg3,

[19-carboxy-Nonadecanoyl]-βAla-Peg3-Peg3-Peg3,

[17-carboxy-Heptadecanoyl]-isoLys,

[19-carboxy-Nonadecanoyl]-isoLys,

[17-carboxy-Heptadecanoyl]-[4-aminobutanoyl]

[19-carboxy-Nonadecanoyl]-[4-aminobutanoyl]

[17-carboxy-Heptadecanoyl]-isoLys-Peg3,

[19-carboxy-Nonadecanoyl]-isoLys-Peg3,

[17-carboxy-Heptadecanoyl]-[4-aminobutanoyl]-Peg3,

[19-carboxy-Nonadecanoyl]-[4-aminobutanoyl]-Peg3,

[17-carboxy-Heptadecanoyl]-isoLys-Peg3-Peg3,

[19-carboxy-Nonadecanoyl]-isoLys-Peg3-Peg3,

[17-carboxy-Heptadecanoyl]-[4-aminobutanoyl]-Peg3-Peg3,

[19-carboxy-Nonadecanoyl]-[4-aminobutanoyl]-Peg3-Peg3,

[17-carboxy-Heptadecanoyl]-isoLys-Peg3-Peg3-Peg3,

[19-carboxy-Nonadecanoyl]-isoLys-Peg3-Peg3-Peg3,

[17-carboxy-Heptadecanoyl]-[4-aminobutanoyl]-Peg3-Peg3-Peg3,

[19-carboxy-Nonadecanoyl]-[4-aminobutanoyl]-Peg3-Peg3-Peg3.

More preferred substituents Z¹—Z²— include:

[Hexadecanoyl]-isoGlu,

[Hexadecanoyl]-βAla,

[Hexadecanoyl]-isoGlu-Peg3,

[Hexadecanoyl]-βAla-Peg3,

[Hexadecanoyl]-isoGlu-Peg3-Peg3,

[Hexadecanoyl]-isoLys,

[Hexadecanoyl]-isoLys-Peg3,

[Hexadecanoyl]-isoLys-Peg3-Peg3,

[17-carboxy-Heptadecanoyl]-isoGlu,

[19-carboxy-Nonadecanoyl]-isoGlu,

[17-carboxy-Heptadecanoyl]-isoGlu-Peg3,

[19-carboxy-Nonadecanoyl]-isoGlu-Peg3,

[17-carboxy-Heptadecanoyl]-isoGlu-Peg3-Peg3,

[19-carboxy-Nonadecanoyl]-isoGlu-Peg3-Peg3,

[17-carboxy-Heptadecanoyl]-isoGlu-Peg3-Peg3-Peg3,

[19-carboxy-Nonadecanoyl]-isoGlu-Peg3-Peg3-Peg3,

[17-carboxy-Heptadecanoyl]-isoLys,

[19-carboxy-Nonadecanoyl]-isoLys,

[17-carboxy-Heptadecanoyl]-isoLys-Peg3,

[19-carboxy-Nonadecanoyl]-isoLys-Peg3,

[17-carboxy-Heptadecanoyl]-isoLys-Peg3-Peg3,

[19-carboxy-Nonadecanoyl]-isoLys-Peg3-Peg3,

[17-carboxy-Heptadecanoyl]-isoLys-Peg3-Peg3-Peg3,

[19-carboxy-Nonadecanoyl]-isoLys-Peg3-Peg3-Peg3.

Yet further preferred substituents Z¹—Z²— include:

[Hexadecanoyl]-KEK, [Octadecanoyl]-KEK,

[Hexadecanoyl]-βAla-Peg3,

[Hexadecanoyl]-KEK-Peg3,

[Hexadecanoyl]-KEK-Peg3-Peg3,

[Hexadecanoyl]-KEK-Peg3-Peg3-Peg3,

[17-carboxy-Heptadecanoyl]-KEK,

[19-carboxy-Nonadecanoyl]-KEK,

[17-carboxy-Heptadecanoyl]-KEK-Peg3,

[19-carboxy-Nonadecanoyl]-KEK-Peg3,

[17-carboxy-Heptadecanoyl]-KEK-Peg3-Peg3,

[19-carboxy-Nonadecanoyl]-KEK-Peg3-Peg3,

[17-carboxy-Heptadecanoyl]-isoGlu-KEK

[19-carboxy-Nonadecanoyl]-isoGlu-KEK,

[17-carboxy-Heptadecanoyl]-isoLys-KEK

[19-carboxy-Nonadecanoyl]-isoLys-KEK,

[17-carboxy-Heptadecanoyl]-βAla-KEK

[19-carboxy-Nonadecanoyl]-βAla-KEK, [17-carboxy-Heptadecanoyl]-KEK-Peg3-Peg3-Peg3,

[19-carboxy-Nonadecanoyl]-KEK-Peg3-Peg3-Peg3,

[17-carboxy-Heptadecanoyl]-[4-aminobutanoyl]-KEK,

[19-carboxy-Nonadecanoyl]-[4-aminobutanoyl]-KEK,

[17-carboxy-Heptadecanoyl]-[4-aminobutanoyl]-KEK-Peg3-Peg3-Peg3,

[19-carboxy-Nonadecanoyl]-[4-aminobutanoyl]-KEK-Peg3-Peg3-Peg3,

[17-carboxy-Heptadecanoyl]-[4-aminobutanoyl]-KEK-Peg3-Peg3,

[19-carboxy-Nonadecanoyl]-[4-aminobutanoyl]-KEK-Peg3-Peg3,

[Hexadecanoyl]-isoGlu-KEK-Peg3,

[Hexadecanoyl]-isoGlu-KEK-Peg3-Peg3,

[19-carboxy-Nonadecanoyl]-isoGlu-KEK,

[19-carboxy-Nonadecanoyl]-[4-aminobutanoyl]-KEK,

[17-carboxy-Heptadecanoyl]-[4-aminobutanoyl]-KEK-Peg3-Peg3-Peg3,

[17-carboxy-Heptadecanoyl]-[4-aminobutanoyl]-KEK-Peg3-Peg3,

[19-carboxy-Nonadecanoyl]-[4-aminobutanoyl]-KEK-Peg3-Peg3,

[17-carboxy-Heptadecanoyl]-KEK-Peg3-Peg3,

[19-carboxy-Nonadecanoyl]-KEK-Peg3-Peg3,

[17-carboxy-Heptadecanoyl]-isoGlu-KEK-Peg3,

[19-carboxy-Nonadecanoyl]-isoGlu-KEK-Peg3,

[17-carboxy-Heptadecanoyl]-isoGlu-KEK-Peg3-Peg3,

[19-carboxy-Nonadecanoyl]-isoGlu-KEK-Peg3-Peg3.

Examples of ψ comprising different substituents (fatty acids, FA), conjugated to the amino acid side-chain, optionally by a spacer, are illustrated below:

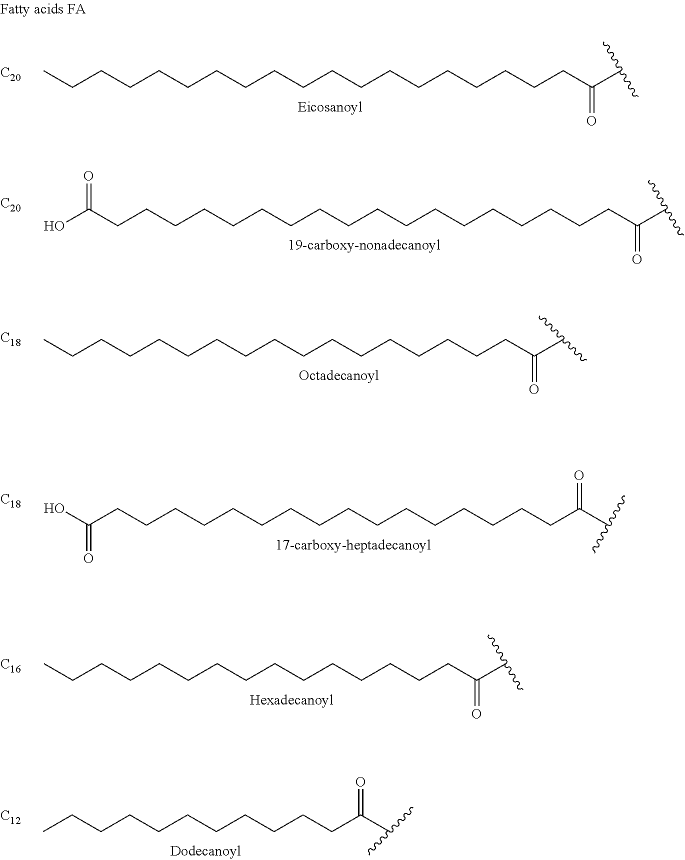

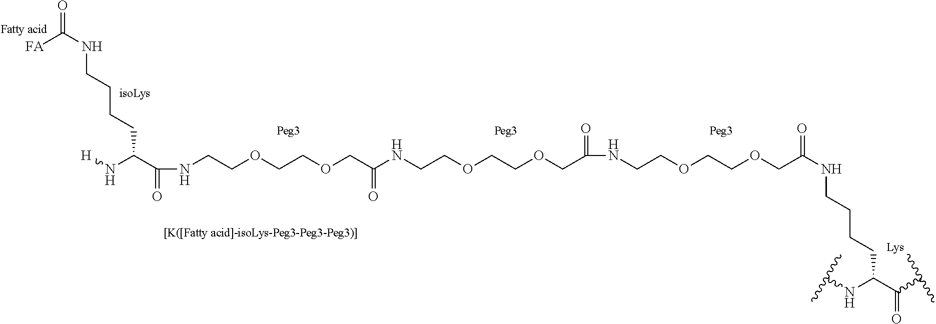

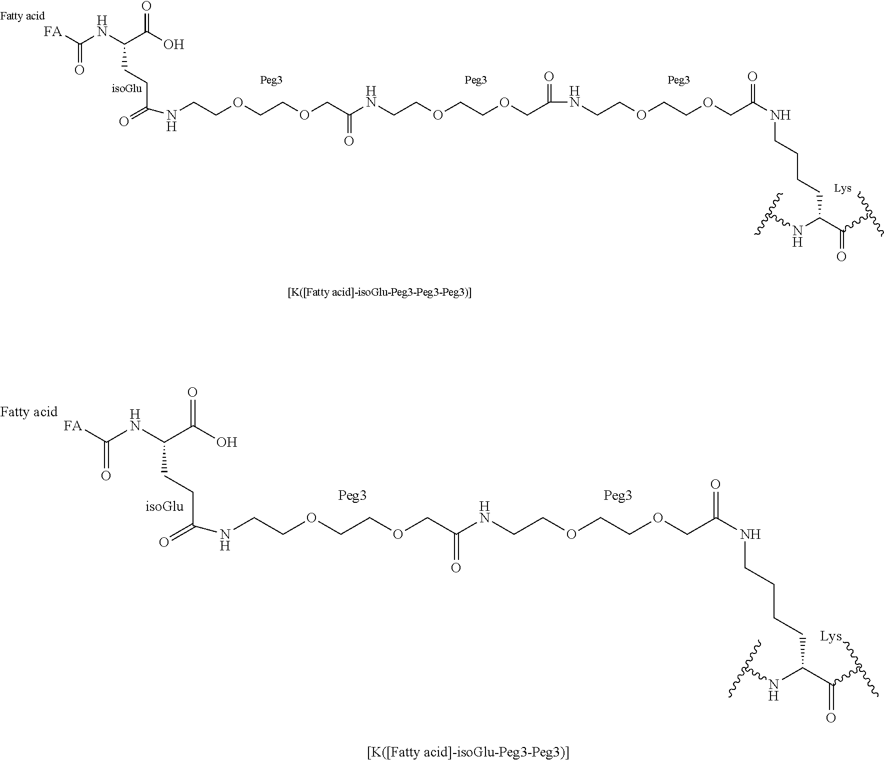

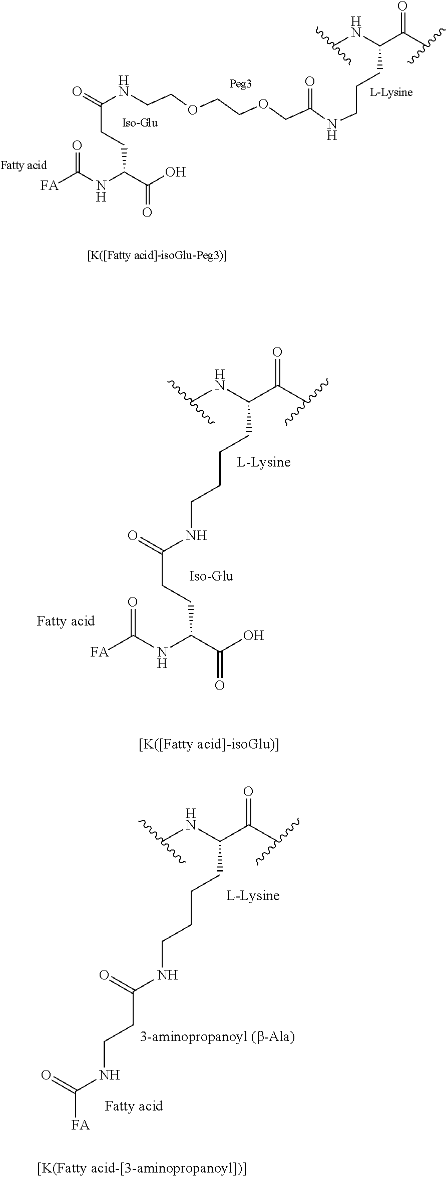

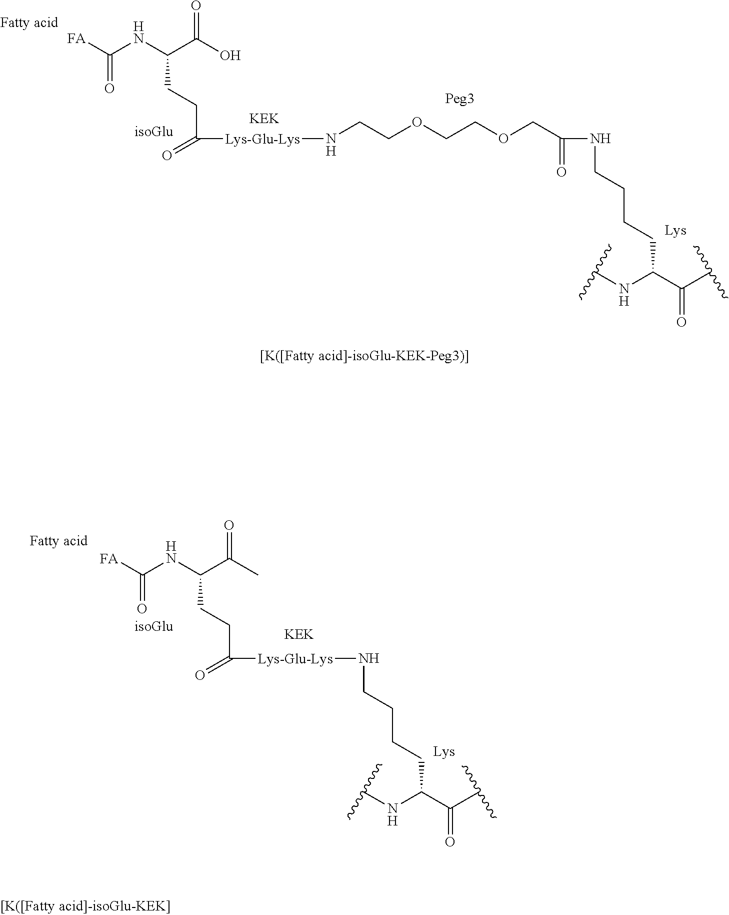

Furthermore, the substituent [Hexadecanoyl]-isoGlu, conjugated to the side chain of a lysine residue, is illustrated below:

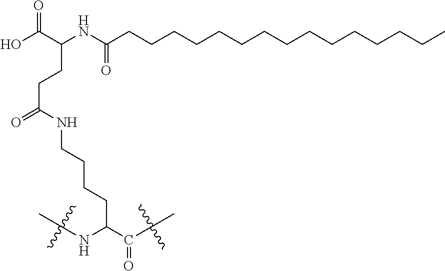 [SMILES: *NC(CCCCNC(=O)CCC(NC(=O)CCCCCCCCCCCCCCC)C(=O)O)C(*)=O]

Thus, the side chain of the Lys residue is covalently attached to the side-chain carboxyl group of the isoGlu spacer —Z2-(—Z^{S1}—) via an amide linkage. A hexadecanoyl group (Z¹) is covalently attached to the amino group of the isoGlu spacer via an amide linkage.

The substituent [Hexadecanoyl]-[4-aminobutanoyl]-conjugated to the side chain of a lysine residue, is illustrated below

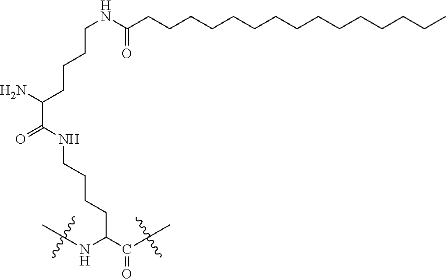 [SMILES: *NC(CCCCNC(=O)C(N)CCCCNC(=O)CCCCCCCCCCCCCCC)C(*)=O]

The substituent [(Hexadecanoyl)iso-Lys]-conjugated to the side chain of a lysine residue, is illustrated below

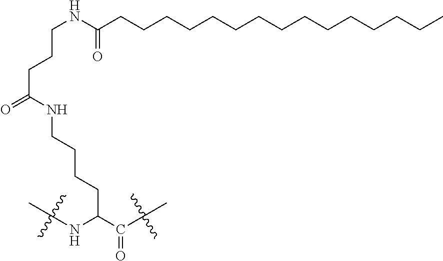 [SMILES: *NC(CCCCNC(=O)CCCNC(=O)CCCCCCCCCCCCCCC)C(*)=O]

The substituent [(Hexadecanoyl)β-Ala]-conjugated to the side chain of a lysine residue, is illustrated below

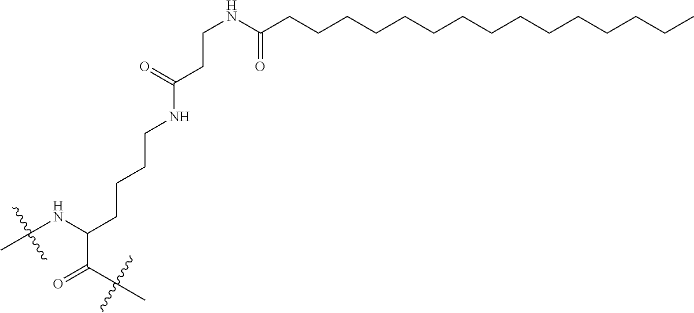 [SMILES: *NC(CCCCNC(=O)CCNC(=O)CCCCCCCCCCCCCCC)C(*)=O]

Some further specific examples of —Z²—Z¹ combinations are illustrated below. In each case, — indicates the point of attachment to the side chain of the amino acid component of ψ:

[17-Carboxy-heptadecanoyl]-isoGlu-Peg3-Peg3

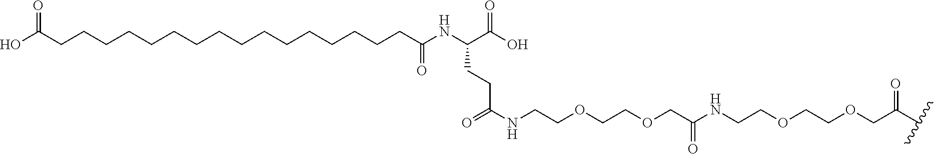 [SMILES: *C(=O)COCCOCCNC(=O)COCCOCCNC(=O)CC[C@H](NC(=O)CCCCCCCCCCCCCCCCC(=O)O)C(=O)O]

[17-Carboxy-heptadecanoyl]-isoGlu

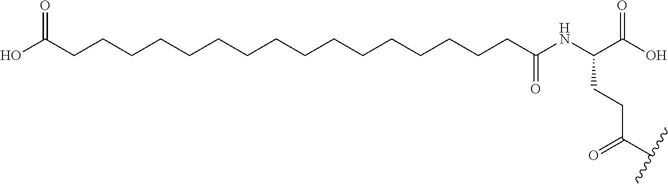 [SMILES: *C(=O)CC[C@H](NC(=O)CCCCCCCCCCCCCCCCC(=O)O)C(=O)O]

[17-carboxy-heptadecanoyl]-iso-Lys-Peg3

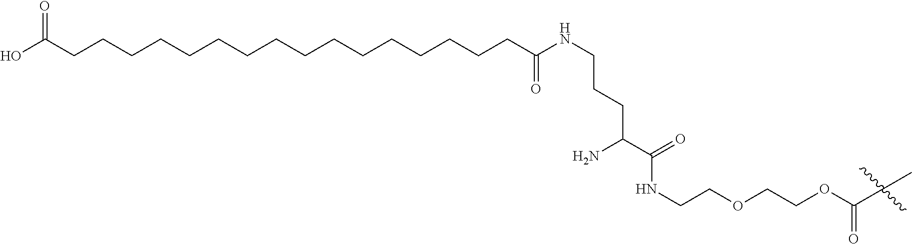 [SMILES: *C(=O)OCCOCCNC(=O)C(N)CCCNC(=O)CCCCCCCCCCCCCCCCC(=O)O]

[17-carboxy-heptadecanoyl]-13-Ala-Peg3

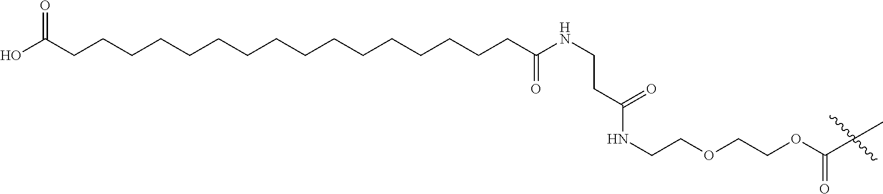 [SMILES: *C(=O)OCCOCCNC(=O)CCNC(=O)CCCCCCCCCCCCCCCCC(=O)O]

4-[17-carboxy-heptadecanoyl]aminobutanoyl-Peg3

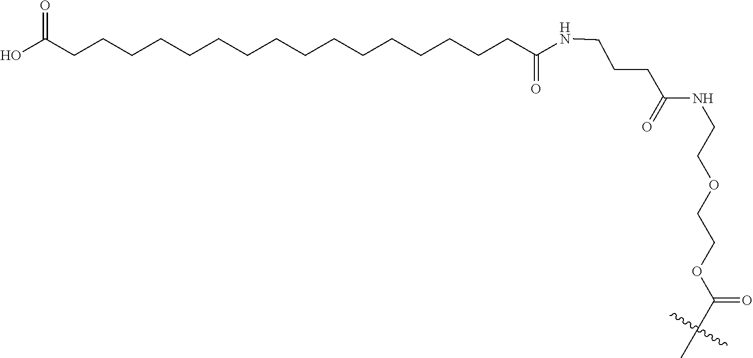 [SMILES: *C(=O)OCCOCCNC(=O)CCCNC(=O)CCCCCCCCCCCCCCCCC(=O)O]

[17-carboxy-heptadecanoyl]-KEK-isoGlu-Peg3-Peg3

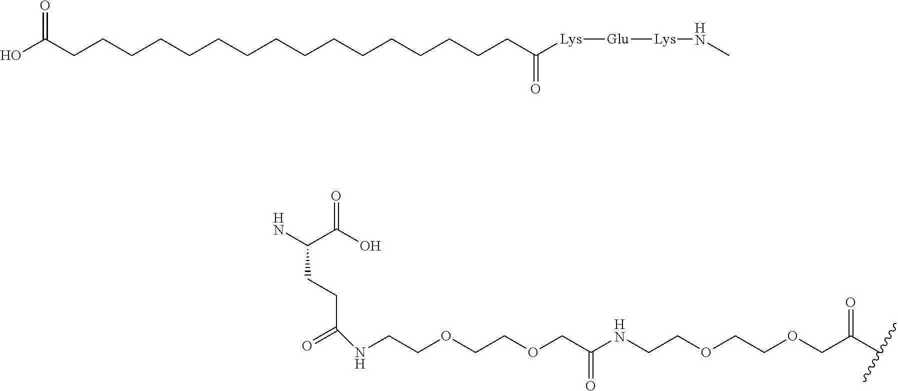

[17-carboxy-heptadecanoyl]-isoGlu-KEK-Peg3-Peg3

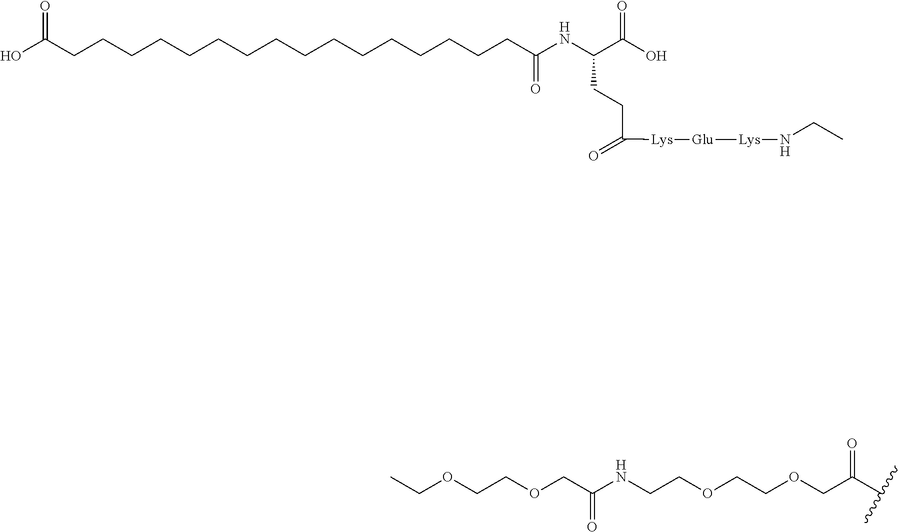

The skilled person will be well aware of suitable techniques for preparing the substituents employed in the context of the invention and conjugating them to the side chain of the appropriate amino acid in the dual agonist peptide. For examples of suitable chemistry, see WO98/08871, WO00/55184, WO00/55119, Madsen et al., J. Med. Chem. 50:6126-32 (2007), and Knudsen et al., J. Med Chem. 43:1664-1669 (2000), incorporated herein by reference.

In another aspect of the invention the dual agonist may be any of the agonists as described in WO2018/104560, which is incorporated herein by reference.

For example, the GLP-1/GLP-2 dual agonist may be a compound represented by the formula:

R¹—X*-U—R²

wherein:

R¹ is hydrogen (Hy), C₁₋₄ alkyl (e.g. methyl), acetyl, formyl, benzoyl or trifluoroacetyl;

R² is NH₂ or OH;

X* is a peptide of formula I:

H-X2-EG-X5-F-X7-X8-E-X10-X11-TIL-X15-X16-X17-A-X19-X20-X21-FI-X24-WL-X27-X28-X29-KIT-X33 (I)

wherein

X2 is Aib or G;

X5 is S or T;

X7 is S or T;

X8 is S, E or D;

X10 is L, M or V;

X11 is A, N or S;

X15 is D or E

X16 is E, A or G;

X17 is Q, E, L or K;

X19 is A, V or S;

X20 is R or K;

X21 is D, L or E;

X24 is A, Nor S;

X27 is I, Y, Q, H or K;

X28 is A, E, H, Y, L, K, Q, R or S;

X29 is H, Y, K or Q;

X33 is D or E;

U is absent or a sequence of 1-15 residues, each independently selected from K and k; and wherein at least one of X5 and X7 is T;

or a pharmaceutically acceptable salt or solvate thereof.

Synthesis of Dual Agonists

The dual agonists according to the invention may be synthesized according to the methods set out in International publication numbers WO2018/104560 and WO2018/104561, which are incorporated herein by reference.

The dual agonists according to the invention may be synthesized according to the methods set out in International publication number WO2018/104561, which is incorporated herein by reference.

Dual agonists may be synthesised by means of solid-phase or liquid-phase peptide synthesis methodology. In this context, reference may be made to WO 98/11125 and, among many others, Fields, G.B. et al., 2002, “Principles and practice of solid-phase peptide synthesis”. In: Synthetic Peptides (2 nd Edition), and the Examples herein.

The dual agonist may be synthesized or produced in a number of ways, including for example, a method which comprises

(a) synthesizing the dual agonist by means of solid-phase or liquid-phase peptide synthesis methodology and recovering the synthesized dual agonist thus obtained; or

(b) expressing a precursor peptide sequence from a nucleic acid construct that encodes the precursor peptide, recovering the expression product, and modifying the precursor peptide to yield a compound of the invention.

The precursor peptide may be modified by introduction of one or more non-proteinogenic amino acids, e.g. Aib, Orn, Dap, or Dab, introduction of a lipophilic substituent Z¹ or Z¹—Z²— at a residue ψ, introduction of the appropriate terminal groups R¹ and R², etc.

Expression is typically performed from a nucleic acid encoding the precursor peptide, which may be performed in a cell or a cell-free expression system comprising such a nucleic acid.

Analogues may be synthesised by means of solid-phase or liquid-phase peptide synthesis. In this context, reference is made to WO 98/11125 and, among many others, Fields, GB et al., 2002, “Principles and practice of solid-phase peptide synthesis”. In: Synthetic Peptides (2 nd Edition).

For recombinant expression, the nucleic acid fragments encoding the precursor peptide will normally be inserted in suitable vectors to form cloning or expression vectors. The vectors can, depending on purpose and type of application, be in the form of plasmids, phages, cosmids, mini-chromosomes, or virus, but also naked DNA which is only expressed transiently in certain cells is an important vector. Preferred cloning and expression vectors (plasmid vectors) are capable of autonomous replication, thereby enabling high copy-numbers for the purposes of high-level expression or high-level replication for subsequent cloning.

In general outline, an expression vector comprises the following features in the 5′—>3′ direction and in operable linkage: a promoter for driving expression of the nucleic acid fragment, optionally a nucleic acid sequence encoding a leader peptide enabling secretion (to the extracellular phase or, where applicable, into the periplasma), the nucleic acid fragment encoding the precursor peptide, and optionally a nucleic acid sequence encoding a terminator. They may comprise additional features such as selectable markers and origins of replication. When operating with expression vectors in producer strains or cell lines it may be preferred that the vector is capable of integrating into the host cell genome. The skilled person is very familiar with suitable vectors and is able to design one according to their specific requirements.

The vectors may be used to transform host cells to produce the precursor peptide. Such transformed cells can be cultured cells or cell lines used for propagation of the nucleic acid fragments and vectors, and/or used for recombinant production of the precursor peptides.

Preferred transformed cells are micro-organisms such as bacteria [such as the species Escherichia (e.g. E. coil), Bacillus (e.g. Bacillus subtilis), Salmonella, or Mycobacterium (preferably non-pathogenic, e.g. M. bovis BCG), yeasts (e.g., Saccharomyces cerevisiae and Pichia pastoris), and protozoans. Alternatively, the transformed cells may be derived from a multicellular organism, i.e. it may be fungal cell, an insect cell, an algal cell, a plant cell, or an animal cell such as a mammalian cell. For the purposes of cloning and/or optimised expression it is preferred that the transformed cell is capable of replicating the nucleic acid fragment of the invention. Cells expressing the nucleic fragment can be used for small-scale or large-scale preparation of the peptides of the invention.

When producing the precursor peptide by means of transformed cells, it is convenient, although far from essential, that the expression product is secreted into the culture medium.

Pharmaceutical Compositions and Administration

The GLP-1/GLP-2 dual agonist according to the present invention may be in the form of a composition comprising a dual agonist, or a pharmaceutically acceptable salt or solvate thereof, together with a carrier. In one embodiment of the invention, the composition is a pharmaceutical composition and the carrier is a pharmaceutically acceptable carrier. The pharmaceutical composition comprising a dual agonist according to the invention, or a salt or solvate thereof, may be together with a carrier, excipient or vehicle. Accordingly, the dual agonist, or salts or solvates thereof, especially pharmaceutically acceptable salts or solvates thereof, may be formulated as compositions or pharmaceutical compositions prepared for storage or administration, and which comprise a therapeutically effective amount of a dual agonist, or a salt or solvate thereof.

Suitable salts formed with bases include metal salts, such as alkali metal or alkaline earth metal salts, for example sodium, potassium or magnesium salts; ammonia salts and organic amine salts, such as those formed with morpholine, thiomorpholine, piperidine, pyrrolidine, a lower mono-, di- or tri-alkylamine (e.g., ethyl-tert-butyl-, diethyl-, diisopropyl-, triethyl-, tributyl- or dimethylpropylamine), or a lower mono-, di- or tri-(hydroxyalkyl)amine (e.g., mono-, di- or triethanolamine). Internal salts may also be formed. Similarly, when a compound of the present invention contains a basic moiety, salts can be formed using organic or inorganic acids. For example, salts can be formed from the following acids: formic, acetic, propionic, butyric, valeric, caproic, oxalic, lactic, citric, tartaric, succinic, fumaric, maleic, malonic, mandelic, malic, phthalic, hydrochloric, hydrobromic, phosphoric, nitric, sulphuric, benzoic, carbonic, uric, methanesulphonic, naphthalenesulphonic, benzenesulphonic, toluenesulphonic, p-toluenesulphonic (i.e. 4-methylbenzene-sulphonic), camphorsulphonic, 2-aminoethanesulphonic, aminomethylphosphonic and trifluoromethanesulphonic acid (the latter also being denoted triflic acid), as well as other known pharmaceutically acceptable acids. Amino acid addition salts can also be formed with amino acids, such as lysine, glycine, or phenylalanine.

In one embodiment, a pharmaceutical composition is one wherein the dual agonist is in the form of a pharmaceutically acceptable acid addition salt.

As will be apparent to one skilled in the medical art, a “therapeutically effective amount” of a dual agonist compound or pharmaceutical composition thereof of the present invention will vary depending upon, inter alia, the age, weight and/or gender of the subject (patient) to be treated. Other factors that may be of relevance include the physical characteristics of the specific patient under consideration, the patient's diet, the nature of any concurrent medication, the particular compound(s) employed, the particular mode of administration, the desired pharmacological effect(s) and the particular therapeutic indication. Because these factors and their relationship in determining this amount are well known in the medical arts, the determination of therapeutically effective dosage levels, the amount necessary to achieve the desired result of treating and/or preventing and/or remedying malabsorption and/or low-grade inflammation described herein, as well as other medical indications disclosed herein, will be within the ambit of the skilled person.

As used herein, the term “a therapeutically effective amount” refers to an amount which reduces symptoms of a given condition or pathology, and preferably which normalizes physiological responses in an individual with that condition or pathology. Reduction of symptoms or normalization of physiological responses can be determined using methods routine in the art and may vary with a given condition or pathology. In one aspect, a therapeutically effective amount of one or more dual agonists, or pharmaceutical compositions thereof, is an amount which restores a measurable physiological parameter to substantially the same value (preferably to within 30%, more preferably to within 20%, and still more preferably to within 10% of the value) of the parameter in an individual without the condition or pathology in question.

The composition for use according to the invention may be administered as a single dose administration. Alternatively, the composition may be administered as a multi dose administration.

Dosing

In one embodiment of the invention, administration of a compound or pharmaceutical composition of the present invention is commenced at lower dosage levels, with dosage levels being increased until the desired effect of preventing/treating the relevant medical indication is achieved. This would define a therapeutically effective amount. For the dual agonists of the present invention, alone or as part of a pharmaceutical composition, such human doses of the active dual agonist may be between about 0. 1 pmol/kg and 500 μmol/kg body weight, between about 0.01 pmol/kg and 300 μmol/kg body weight, between 0.01 pmol/kg and 100 μmol/kg body weight, between 0.1 pmol/kg and 50 μmol/kg body weight, between 1 pmol/kg and 10 μmol/kg body weight, between 5 pmol/kg and 5 μmol/kg body weight, between 10 pmol/kg and 1 μmol/kg body weight, between 50 pmol/kg and 0.1 μmol/kg body weight, between 100 pmol/kg and 0.01 μmol/kg body weight, between 0.001 μmol/kg and 0.5 μmol/kg body weight, between 0.05 μmol/kg and 0.1 μmol/kg body weight.

In one aspect the dose is in the range of about 50 pmol/kg to 500 nmol/kg, for example about 60 pmol/kg to 400 nmol/kg, about 70 pmol/kg to 300 nmol/kg, about 80 pmol/kg to 200 nmol/kg, about 90 pmo1/kg to 100 nmol/kg.

In one aspect the dose is in the nmol range, for example between 1 nmol/kg and 100 nmol/kg, between 1 nmol/kg and 90 nmol/kg, between 1 nmol/kg and 80 nmol/kg, between 1 nmol/kg and 70 nmol/kg, between 1 nmol/kg and 60 nmol/kg, between 1 nmol/kg and 50 nmol/kg, between 1 nmol/kg and 40 nmol/kg, between 1 nmol/kg and 30 nmol/kg, between 1 nmol/kg and 20 nmol/kg, or between 1 nmol/kg and 10 nmol/kg. The therapeutic dosing and regimen most appropriate for patient treatment will of course vary with the disease or condition to be treated, and according to the patient's weight and other parameters. Without wishing to be bound by any particular theory, it is expected that doses, in the pmol/kg or nmol/kg range, and shorter or longer duration or frequency of treatment may produce therapeutically useful results, such as a statistically significant increase particularly in small bowel mass. In some instances, the therapeutic regimen may include the administration of maintenance doses appropriate for preventing tissue regression that occurs following cessation of initial treatment. The dosage sizes and dosing regimen most appropriate for human use may be guided by the results obtained by the present invention, and may be confirmed in properly designed clinical trials.

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person.

In one aspect the dual agonist according to the invention is administered intravenously, in a continuous infusion.

In one aspect the patient is an adult. In one aspect the patient is a neonate. In one aspect the patient is a child.

Clinical Nutrition

Clinical nutrition deals with the prevention, diagnosis, and management of nutritional and metabolic changes related to acute and chronic diseases and conditions caused by a lack or excess of energy and nutrients. Nutrition therapy describes how nutrients are provided to treat any nutritional-related condition. Nutrition or nutrients can be provided orally (regular diet, therapeutic diet, oral nutritional supplements), via enteral tube feeding, or alternatively as parenteral nutrition. Medical nutrition therapy relates to oral nutritional supplements, enteral tube feeding, i.e. enteral nutrition, and parenteral nutrition.

In the present invention the patient is one who is receiving parenteral nutrition.

Parenteral nutrition is a type of nutrition therapy provided through intravenous administration of nutrients such as amino acids, glucose, lipids, vitamins, electrolytes and required trace elements.

Parenteral nutrition may be administered in a broad range of patients with a variety of conditions. The present invention is broadly applicable to any patient who is receiving parenteral nutrition for whatever reason.

Parenteral nutrition may be administered centrally through a central venous line, or alternatively peripherally through a peripheral intravenous line.

Total parenteral nutrition (TPN) (or exclusive parenteral nutrition) refers to the situation where the patient's complete nutritional needs are covered by the parenteral nutrition, and in which nutrition is not given to the patient through any other route.

Supplemental, partial, or complementary parenteral nutrition is supplementary (partial or complementary) nutrition where nutrition is provided in addition to parenteral nutrition by any route other than intravenously. For example, this may be given when the enteral or oral route may not independently achieve the required nutritional needs.

Home parenteral nutrition is when parenteral nutrition is used outside the hospital setting. This is often used for patients with chronic intestinal failure, malignant obstruction or partial obstruction.

Parenteral nutrition may also be given subcutaneously (subcutaneous fluid therapy). This is a special parenteral route primarily used to provide fluids. It can also provide limited amounts of amino acids and glucose when the intravenous route is unavailable.

Intra-dialytic parenteral nutrition is parenteral nutrition given intravenously through the venous line of the dialysis circuit. This is not a routine technique for supplemental nutritional therapy, but may be indicated to prevent nutritional deterioration in patients on dialysis.

The patient according to the present invention may be receiving any type of parenteral nutrition.

Suitable parenteral compositions or formula will be known to one skilled in the art.

Parenteral solutions are composed of carbohydrates (glucose), lipids and amino acids and can include electrolytes, vitamins and trace elements as required. They are defined by the relative composition of the macronutrients, osmolarity, pH and calorie content. These solutions can be administered using separate bottles but are usually administered using compounding or ready to mix bags.

Parenteral nutrition compositions are intended to provide energy and nutrients, rather than hydration alone. They are usually given intravenously. Parenteral compositions can aim to provide a single group of nutrients (e.g. the use of lipid emulsion alone) or a combination of nutrients that is more typically thought of as a PN composition or infusate

A three chamber bag (usually industry manufactured) or all-in-one (mainly pharmacy provided) parenteral infusate is an emulsion in which amino acids, glucose and lipid emulsion are combined in a single infusate, along with electrolytes, vitamins and trace elements as required. Three-chamber bags contain all macronutrients and electrolyte sin three separate compartments. The substrates are mixed together immediately prior to intravenous application by breaking the separation seals between the bag chambers. Three chamber bags are available with or without basic electrolytes. Vitamins and trace elements are injected into the bag prior to administration.

Individually compounded all-in-one (AIO) admixtures allow for the provision of patient-specific ready-to-use parenteral infusates, adapted according to energy, volume and substrate needs. These are aseptically manufactured from various components, usually in hospital pharmacies, and are designed for immediate intravenous administration, with no mixing or admixing required prior to administration. These bags are usually compounded daily or weekly due to their often limited stability.

A two chamber bag (usually industry manufactured) or two-in-one (mainly pharmacy provided) parenteral infusate is a solution in which amino acids and glucose (no lipid emulsion) are combined in a single infusate, along with electrolytes, vitamins and trace elements as required. Two-in-one parenteral infusates may be required if a formulation is pharmaceutically unstable when lipid emulsion is included, or when the aim is not to provide lipids.

A parenteral nutrition component is intended to be combined with other PN components to formulate the requirements of a prescription for PN. Individual products must be intended for parenteral use and must be combined in a suitable environment and under aseptic techniques that ensures sterility of the final product. In some cases, PN components are administered independently, except for Water for Injection.

Commercial crystalline amino acid solutions contain a mixture of different concentrations and profile of crystalline amino acids, and are available with or without the inclusion of electrolytes.

Commercial glucose solutions contain glucose in Water for Injection at different concentrations, typically from 5% w/v up to 70% w/v. A concentration of 12.5% w/v is considered to be a limit to avoid complications from peripheral administration.

Commercial lipid emulsions are a lipid-in-water emulsion that contains a mixture of triglycerides with different fatty acid chains. For some products, they are available in more than one concentration i.e. 10% w/v, 20% w/v and/or 30% w/v. The products contain the essential fatty acids, i.e. linolenic and linoleic acids, mainly derived from soy bean oil. There are several oils used in the production of lipid emulsions for intravenous administration. Other lipid sources include olive oil or fish oil. Soy bean, olive and fish oil provide long chain fatty acids (LCT),whereas coconut oil provides medium chain triglycerides (MCT).

Water for Injection contains no components other than sterile water suitable for parenteral administration. An electrolyte solution consists of an electrolyte salt in Water for Injection. Many are available indifferent volumes, concentrations, different units of concentration, types of container (e.g. glass or plastic), or with the intended electrolyte available as different salts.

For parenteral nutrition a standard dosage of vitamins and trace elements is generally recommended because individual requirements cannot be easily determined. Preferably, all vitamins and trace elements supplied with a normal diet should also be substituted with parenteral nutrition as available.

In one aspect the invention is effective to reduce or eliminate a need for the patient to receive parenteral nutrition or facilitate the patient's return to enteral feeding. In one aspect the reduction in parenteral nutrition is 5, 10, 15, 20, 25, 30, 35, 40, 45, 50, 55, 60, 65, 70, 75, 80, 85, 90, 95 or 100%.

Enteral tube feeding or nutrition is nutrition therapy given via a tube or stoma into the intestinal tract distal to the oral cavity. The tube may be inserted via the nose (naso-gastric, naso-jejunal or naso-post pyloric tube feeding). Enteral nutrition may alternatively be delivered via a stoma that is inserted endoscopically into the stomach (percutaneous endoscopic gastrostomy (PEG)) or with a jejunal extension (PEG-J) or into the jejunum (percutaneous endoscopic jejunostomy (PEJ)). Alternatively, the tube may also be placed surgically (surgical gastrostomy or jejunostomy).

Total enteral tube feeding, or total enteral nutrition, denotes the situation where all nutrient needs are provided through a feeding tube without significant oral or parenteral intake. Supplemental enteral tube feeding denotes nutrition given to patients whose oral intake of food and fluids is inadequate for reaching their defined nutritional target alone. When enteral feeding is used outside the hospital it is called home enteral nutrition (HEN) or home enteral tube feeding (HETF). HEN/H ETF may be provided either as total or supplemental enteral nutrition

Suitable enteral nutrition compositions or formulas will be known to one of skill in the art. Nutrition products that are delivered via enteral feeding are defined in EU legislation as “foods for special medical purposes” (FSMPs). Such products are specially processed or formulated and intended for the dietary management of patients under medical supervision.

By way of example, standard formulas (whole protein formulas) are designed for adults or children who have normal digestion. Standard formulas include all of the nutrients required to maintain health. Some standard formulas can be used for both enteral feeding and as an oral supplement. They can contain added ingredients, such as fibre, for digestive health and bowel management.

Peptide formulas (semi-elemental formulas) are nutritionally complete, which means they contain all the essential nutrients needed. However, unlike standard formulas, some of the components, such as protein are “broken down” into smaller components to make them easier to digest. Peptide formulas are easier for the digestive system to digest and absorb, making them better suited for adults and children with digestive problems, including malabsorption, short bowel syndrome, inflammatory bowel disease, cystic fibrosis and other conditions that can cause problems with absorbing nutrients.

Specialised enteral formulas are also available for adults and children with special nutritional needs, such as diabetes, kidney failure, respiratory disease, or liver disorders. The enteral formula should be selected by a doctor or a dietitian who is familiar with the various formulas.

Various enteral nutritional compositions are available with different contents of nutrition, depending on the patient's needs and clinical situation. Examples of commercially available enteral nutritional compositions include: Isocal (Novartis), Nutren 1.0 (Nestle), Osmolite 1.0 (Ross), Fibersource 1.2 (Novartis), Jevity 1.2 (Ross), Osmolite 1.2 (Ross), probalance (Nestle), Isosource 1.5 (Novartis), Jevity 1.5 (Ross), Nutren 1.5 (Nestle), Deliver 2.0 (Novartis), Novasource 2.0 (Novartis), Nutren 2.0 (Nestle) and TwoCal HN (Ross).

For example, the protein content of the composition may be from 10 to 80%. The protein component may be made with casein, soy, hydrolyzed protein with added amino acids, or free amino acids alone.

The carbohydrate content of the composition may be from 10 to 90%. The carbohydrate component may be made with starch, glucose polymers, and/or disaccharides such as sucrose.

The fat content may be from 10% to 50%. The fat content may be made with long-chain triglycerides, medium chain triglycerides and fish or other specialty oils

A standard enteral nutritional composition may contain the following:
Protein content: 10-15%
Carbohydrate content: 50-60%
Fat content: 30-35%

In one aspect the enteral formula is free of lactose and/or gluten.

In one aspect the amount of enteral nutrition the patient is able to receive increases, for example to 5, 10, 15, 20, 25, 30, 35, 40, 45, 50, 55, 60, 65, 70, 75, 80, 85, 90, 95 or 100% of the recommended daily intake.

Nutrient concentrations of standard enteral nutrition compositions may vary from 1.0-2.0 kcal/mL. In general, energy, protein and micronutrient needs are covered by 1.5L of standard enteral formula.

In one aspect said enteral nutrition may provide to the patient at least about 200, 300, 400, 500, 600, 700, 800, 900 or more kcal per day. In one aspect said enteral nutrition may provide to the patient at least about 1000 kcal per day. For example, said enteral nutrition may provide at least about 1100 1200, 1300, 1400, 1500, 1600, 1700, 1800, 1900, 2000, 2100, 2200, 2300, 2400 or 2500 kcal per day.

Treatments

The present invention may be used to increase intestinal mass, improve intestinal function, increase intestinal blood flow, or repair intestinal damage or dysfunction (whether structural or functional), e.g. damage to the intestinal epithelium. The invention may also be used in the prophylaxis or treatment of conditions which may be ameliorated by these effects, and in reducing the morbidity related to gastrointestinal damage.

In one aspect the invention may be used for the treatment of a patient who has intestinal insufficiency or failure.

In one aspect the invention may be used for the treatment of a patient who has hepatic impairment or insufficiency.

In one aspect the invention may be used for prophylaxis or treatment of malabsorption, ulcers (e.g. peptic ulcers, Zollinger-Ellison Syndrome, drug-induced ulcers, and ulcers related to infections or other pathogens), short-bowel syndrome, cul-de-sac syndrome, inflammatory bowel disease (Crohns disease and ulcerative colitis), irritable bowel syndrome (IBS), pouchitis, celiac sprue (for example arising from gluten induced enteropathy or celiac disease), tropical sprue, hypogammaglobulinemic sprue, mucositis induced by chemotherapy or radiation therapy, diarrhea induced by chemotherapy or radiation therapy, low grade inflammation, metabolic endotoxemia, necrotising enterocolitis, primary biliary cirrhosis, hepatitis, fatty liver disease (including parental nutrition associated gut atrophy, PNALD (Parenteral Nutrition-Associated Liver Disease), NAFLD (Non-Alcoholic Fatty Liver Disease) and NASH (Non-Alcoholic Steatohepatitis)), or gastrointestinal side-effects of inflammatory conditions such as pancreatitis or graft versus host disease (GVHD).

Short Bowel Syndrome (SBS)

In one aspect of the invention the patient has short bowel syndrome (SBS).

SBS usually results from surgical resection of some or most of the small intestine for conditions such as Crohn's disease, mesenteric infarction, volvulus, trauma, congenital anomalies, and multiple strictures due to adhesions or radiation.

SBS patients suffer from malabsorption that may lead to malnutrition, dehydration and weight loss. Some patients can maintain their protein and energy balance through hyperphagia; more rarely they can sustain fluid and electrolyte requirements to become independent from parenteral fluid.

Short bowel syndrome (SBS) is anatomically defined as that symptom complex which occurs in adults who have less than 200 centimeters of combined jejunum-ileum following small bowel resection. As the intestinal length in children is linked to the state of growth, a definition of a short bowel in absolute terms has not been devised. The need for intravenous supplementation or a residual short bowel length of less than 25% expected for gestational age are suggested definitions of a short bowel in children

The syndrome is characterized by diarrhea, weight loss, dehydration, malnutrition, and malabsorption of macro- and micronutrients. Note that some patients who have had extensive bowel resections leaving them with more than 200 centimeters of small intestine can develop symptoms indistinguishable from those who fulfill the technical criteria for SBS. This situation occurs when the remaining bowel is diseased as, for example, in patients with Crohn's disease or radiation enteritis. These latter patients are generally managed like the standard SBS patient.

Advantageous Effects

As described herein, the present invention is advantageous in that it may ameliorate unwanted side effects associated with parenteral nutrition.

As demonstrated in the present Examples, the composition according to the present invention may lead to increased intestinal mass and/or an increase in the villous/crypt ratio in the intestine. As such, the invention may lead to improved properties or function of the intestinal mucosa of patients who are receiving parenteral nutrition.

In one aspect the invention may decrease intestinal atrophy in the patient, and/or increase the patient's ability to absorb water, energy and other nutrients

As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In one aspect the composition for use according to the present invention may lead to increased intestinal mass. The increase in mass of the intestine in patients receiving the composition may be about 5, 10, 15, 20, 25, 30, 35, 40, 45, 50, 55, 60, 65, 70, 75, 80, 85, 90, 95 or 100% compared to patients to whom the composition is not administered.

In one aspect the villus to crypt ratio is increased. In one aspect growth of the villi is increased. In one aspect the increase may be about 5, 10, 15, 20, 25, 30, 35, 40, 45, 50, 55, 60, 65, 70, 75, 80, 85, 90, 95 or 100%.

One skilled in the art will be aware of how to measure such parameters using techniques known in the art. Standard techniques and methods may be used to measure such parameters, such as those described in the present Examples, e.g. histological techniques and visual measurement and assessment, which will be known to one skilled in the art.

Methods of Treatment

The present invention encompasses methods of treatment and therapeutic uses corresponding to all of aspects of the composition for use as described herein.

As such, the invention provides a method for treating a patient who is receiving parenteral nutrition, wherein said method comprises administering a GLP-1/GLP-2 dual agonist as described herein to said patient.

The invention also encompasses the use of a GLP-1/GLP-2 dual agonist as described herein in the manufacture of a medicament for use in the treatment of a patient who is receiving parenteral nutrition.

The invention also provides use of a GLP-1/GLP-2 dual agonist as described herein in the treatment of a patient who is receiving parenteral nutrition.

Methods of treatment corresponding to all aspects of the dual agonist or composition for use as described herein are encompassed by the present invention. Similarly, uses corresponding to all aspects of the dual agonist or composition for use as described herein are encompassed by the present invention.

In a further aspect there is provided a kit comprising a composition comprising a dual agonist according to the invention for use in the treatment of a patient who is receiving parenteral nutrition.

The following examples are provided to illustrate preferred aspects of the invention and are not intended to limit the scope of the invention in any way.

### EXAMPLES

### Example 1: Effect of Compound 18 in Total Parental Nutrition (TPN) Piglet Model

Animals, Nutrition and Treatment

One-week-old piglets were placed in heated (25-30° C.) cages. Jugular and duodenal catheters were surgically implanted as described in paragraph 2 (method and material) in Jain 2015. Subsequently, preconditioned jackets and ambulatory pumps for TPN (total parenteral nutrition) delivery were fitted. Animals were started on their randomly assigned groups to receive enteral nutrition (EN; n=11), intravenous total parenteral nutrition plus vehicle (TPN; n=8) or TPN plus compound 18 (Cpd 18; n=13). Cpd 18 and vehicle were administered as daily subcutaneous injections at a dose of either 0.008 mg/kg (n=2), 0.033 mg/kg (n=2), 0.066 mg/kg (n=2) or 0.133 mg/kg (n=7).

Enteral animals (EN) received a swine milk replacement formula (formula LitterLife as described in Jain 2015, paragraph 2.6). All other animals received total parenteral nutrition (Clinimix E from Baxter as described in Jain 2015, paragraph 2.6).

Tissue Collection

Post-euthanasia (see Jain 2015, paragraph 2.7), the abdomen was opened, and the small intestine was removed. Small segments of the small bowel were sliced and weighed (see Jain 2015, paragraph 2.78). Tissue was then cut into smaller pieces, preserved for histology and snap-frozen in liquid nitrogen to be stored at −80° C. for analysis.

Histology

Segments of fresh tissue (2-3 cm) from the small intestine were fixed in 4% buffered formalin for 24 hours and then stored in 70% ethanol at room temperature for 24 hours. The tissue was then processed, embedded in paraffin, and stained for hematoxylin and eosin (H&E). An automated upright microscope system with LED illumination (Life Sciences Leica DM4000 B LED) was used along with Q-capture pro digital imaging software was used for capturing images and perform the histology analyses. Using the small-bowel H&E slides, the mean villous height and crypt depth were quantified in vertically well-oriented villous-crypt columns with the slide reviewer blinded to the treatment to compute a villous/crypt (V/C) ratio for animals in each group.

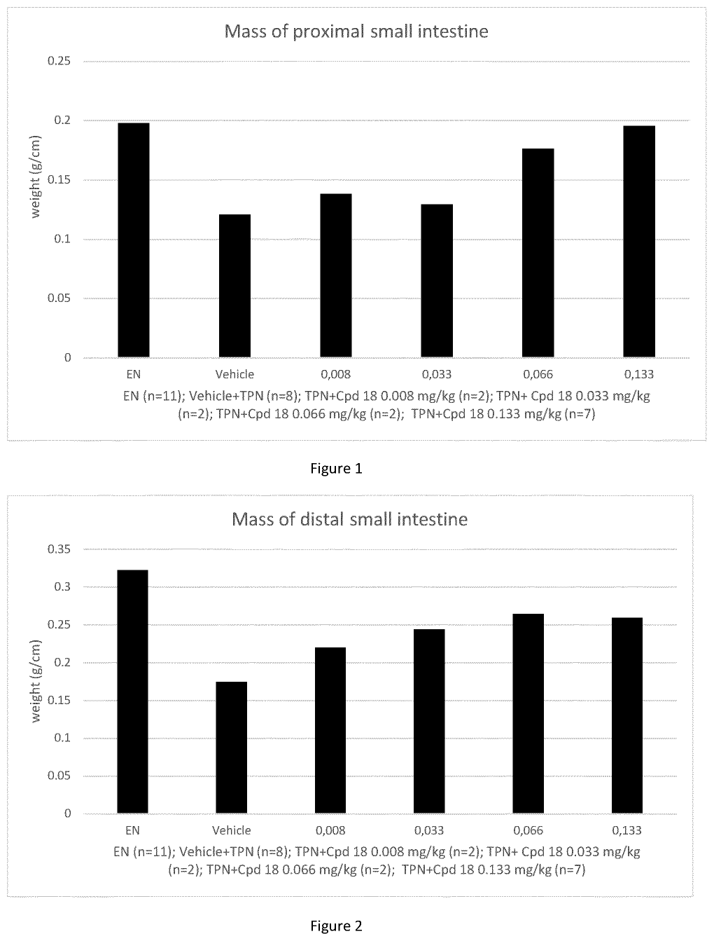

The results show, as seen from FIG. 1, that weight per centimeter determinations at sacrifice on day 14 showed significant reduction in the mass of the proximal intestine in animals on TPN and vehicle when compared to animals on EN. Treatment with TPN and Cpd 18 preserved the mass in a dose-dependent fashion and at the highest dose the weight of the intestine was approximately the same as in the animals on EN.

From FIG. 2 it can be seen that the weight per centimeter determinations at sacrifice on day 14 showed significant reduction in the mass of the distal intestine in animals on TPN and vehicle when compared to animals on EN. Treatment with TPN and Cpd 18 preserved the mass in a dose-dependent fashion and at the highest dose the weight of the intestine was approximately 50% greater than that in the animals on TPN.

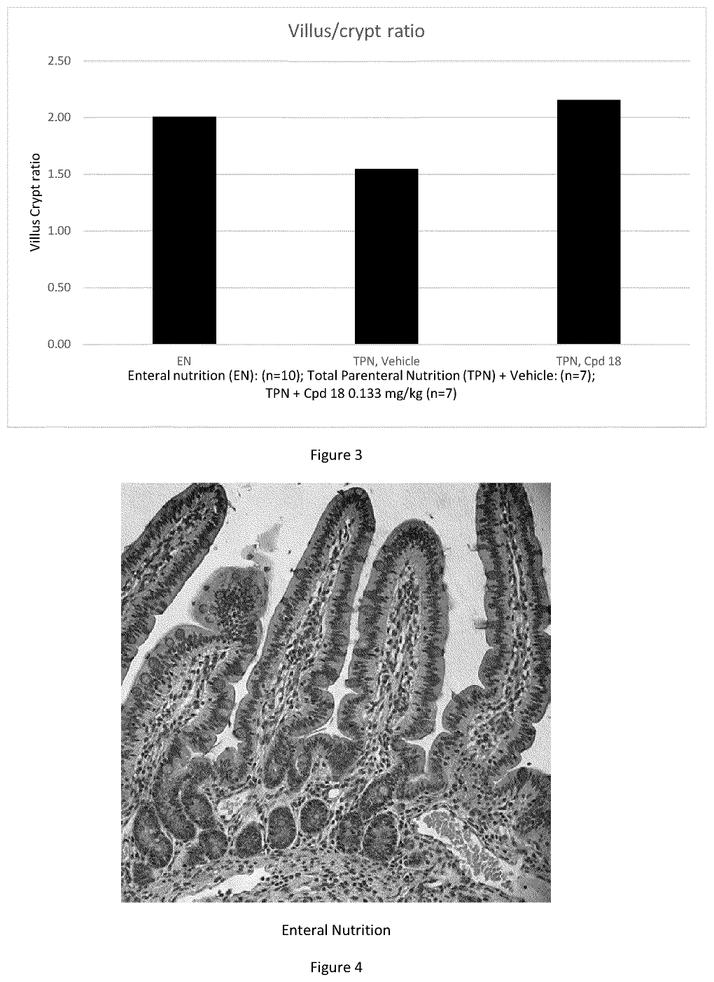

The histological evaluation of the V/C ratio of the small bowel at sacrifice on day 14 showed markedly reduced V/C ratio in animals on TPN and vehicle when compared to animals on EN (FIG. 3). Treatment with TPN and Cpd 18 at a dose 0.133 mg/kg completely preserved the V/C ratio to the same ratio seen in the animals on EN.

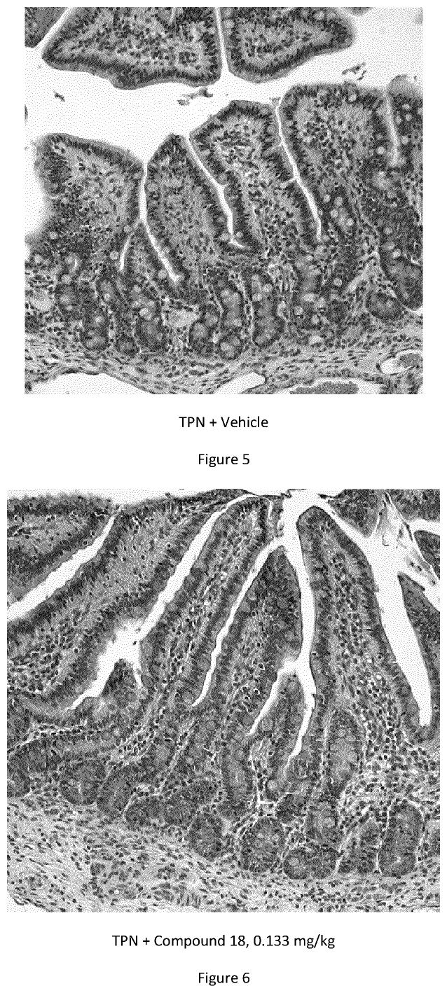

FIGS. 4 shows a representative histological section of the small bowel showing normal villi and crypts in an animal receiving enteral nutrition. A significant villous atrophy in an animal receiving TPN and vehicle for 14 days is seen (FIG. 5). However, in a representative histological section of the small bowel preservation of villi and crypts in an animal receiving TPN and Cpd 18 at a dose of 0.133 mg/kg for 14 days (FIG. 6), villi and crypts are comparable to the animal receiving enteral nutrition (FIG. 4).

### REFERENCES

1. Jain A K, Wen J X, Arora S, et al. Validating hyperbilirubinemia and gut mucosal atrophy with a novel ultramobile ambulatory total parenteral nutrition piglet model. Nutrition research. 2015; 35(2):169-174.

2. Villalona G, Price A, Blomenkamp K, et al. No Gut No Gain! Enteral Bile Acid Treatment Preserves Gut Growth but Not Parenteral Nutrition-Associated Liver Injury in a Novel Extensive Short Bowel Animal Model. JPEN Journal of parenteral and enteral nutrition. 2018; 42(8):1238-1251.

The invention is also described in the following numbered embodiments:

1. A composition comprising a GLP-1/GLP-2 dual agonist for use in the treatment of a patient receiving parenteral nutrition.

2. The composition for use according to embodiment 1 for use in prophylaxis or treatment of malabsorption, ulcers, short-bowel syndrome, cul-de-sac syndrome, inflammatory bowel disease, irritable bowel syndrome, pouchitis, celiac sprue, tropical sprue, hypogammaglobulinemic sprue, mucositis induced by chemotherapy or radiation therapy, diarrhea induced by chemotherapy or radiation therapy, low grade inflammation, metabolic endotoxemia, necrotising enterocolitis, primary biliary cirrhosis, hepatitis, fatty liver disease, or gastrointestinal side-effects of inflammatory conditions.

3. The composition for use according to embodiment 1 or embodiment 2 wherein said patient has intestinal insufficiency or failure.

4. The composition for use according to any preceding embodiment wherein said patient has hepatic impairment or insufficiency.

5. The composition for use according to any preceding embodiment wherein said GLP-1/GLP-2 dual agonist is a peptide.

6. The composition for use according to any preceding embodiment wherein said GLP-1/GLP-2 dual agonist is a compound represented by the formula:

R¹—X*-U—R²

wherein:

R¹ is hydrogen (Hy), 01-4 alkyl (e.g. methyl), acetyl, formyl, benzoyl or trifluoroacetyl;

R² is NH₂ or OH;

X* is a peptide of formula I:

H-X2-EG-X5-F-X7-X8-E-X10-X11-TIL-X15-X16-X17-A-X19-X20-X21-FI-X24-WL-X27-X28-X29-KIT-X33 (I)

wherein:

X2 is Aib or G

X5 is T or S;

X7 is T or S;

X8 is S, E or D;

X10 is L, M, V or ψ;

X11 is A, N or S;

X15 is D or E;

X16 is G, E, A or ψ;

X17 is Q, E, K, L or ψ;

X19 is A, V or S;

X20 is R, K or ψ;

X21 is D, L or E;

X24 is A, Nor S;

X27 is I, Q, K, H or Y;

X28 is Q, E, A, H, Y, L, K, R or S;

X29 is H, Y, K or Q;

X33 is D or E;

U is absent or a sequence of 1-15 residues each independently selected from K, k, E, A, T, I, L and ψ;

the molecule contains one and only one ψ, wherein ψ is a residue of K, k, R, Orn, Dap or Dab in which the side chain is conjugated to a substituent having the formula Z¹— or Z¹—Z²—, wherein

Z¹— is CH₃—(CH₂)₁₀₋₂₂—(CO)— or HOOC—(CH₂)₁₀₋₂₂—(CO)—; and

—Z²— is selected from —Z^{S1}—, —Z^{S1}—Z^{S2}—, —Z^{S2}—Z^{S1}—Z^{S2}—, —Z^{S3}—, —Z^{S1}Z^{S3}—, —Z^{S2}Z^{S3}—, —Z^{S3}Z^{S1}—, —Z^{S3}Z^{S2}—, —Z^{S1}Z^{S2}Z^{S3}—, —Z^{S1}Z^{S3}Z^{S2}—, —Z^{S2}Z^{S1}Z^{S3}—, —Z^{S2}Z^{S3}Z^{S1}—, —Z^{S3}Z^{S1}Z^{S2}—, —Z^{S3}Z^{S2}Z^{S1}—, Z^{S2}Z^{S3}Z^{S2}—

wherein

Z^{S1} is isoGlu, β-Ala, isoLys, or 4-aminobutanoyl;

Z^{S2} is -(Peg3)ₘ- where m is 1, 2, or 3; and

—Z^{S3}— is a peptide sequence of 1-6 amino acid units independently selected from the group consisting of A, L, S, T, Y, Q, D, E, K, k, R, H, F and G;

and wherein at least one of X5 and X7 is T;

or a pharmaceutically acceptable salt or solvate thereof.

7. A dual agonist or pharmaceutically acceptable salt or solvate thereof according to embodiment 6 wherein:

X2 is Aib or G;

X5 is T or S;

X7 is T or S;

X8 is S;

X10 is L or ψ;

X11 is A or S;

X15 is D or E;

X16 is G, E, A or ψ;

X17 is Q, E, K, L or ψ;

X19 is A or S;

X20 is R or ψ;

X21 is D, L or E;

X24 is A;

X27 is I, Q, K, or Y;

X28 is Q, E, A, H, Y, L, K, R or S;

X29 is H, Y or Q; and

X33 is D or E.

8. A dual agonist or pharmaceutically acceptable salt or solvate thereof according to embodiment 6 or embodiment 7 wherein X16 is E and X17 is Q.

9. A dual agonist or pharmaceutically acceptable salt or solvate thereof according to any one embodiments 6 to 8 wherein:

X11 is A and X15 is D;

X11 is S and X15 is E; or

X11 is A and X15 is E.

10. A dual agonist or pharmaceutically acceptable salt or solvate thereof according to any one embodiments 6 to 9 wherein X27 is I.

11. A dual agonist or pharmaceutically acceptable salt or solvate thereof according to any one embodiments 6 to 10 wherein X29 is H, and optionally X28 is A or X28 is E.

12. A dual agonist or pharmaceutically acceptable salt or solvate thereof according to any one of embodiments 6 to 11 wherein X29 is Q and optionally X27 is Q.

13. A dual agonist or pharmaceutically acceptable salt or solvate thereof according to any one of embodiments 6 to 12 wherein the residues at X27-X29 have a sequence selected from:

IQH;

IEH

IAH;

IHH;

IYH;

ILH;

IKH;

IRH;

ISH;

QQH;

YQH;

KQH;

IQQ;

IQY;

IQT; and

IAY.

14. A dual agonist or pharmaceutically acceptable salt or solvate thereof according to embodiment 6 wherein X* is a peptide of formula II:

H-X2-EG-X5-F-X7-SELATILD-X16-X17-AAR-X21-FIAWLI-X28-X29-KITD (II)

wherein:

X2 is Aib or G

X5 is T or S;

X7 is T or S;

X16 is G or ψ;

X17 is Q, E, K, L or ψ;

X21 is D or L;

X28 is Q, E, A, H, Y, L, K, R or S;

X29 is H, Y or Q.

15. A dual agonist or pharmaceutically acceptable salt or solvate thereof according to embodiment 6 or embodiment 14 wherein X16 is 4⁴ and X17 is Q, E, K or L, or X16 is G and X17 is ψ.

16. A dual agonist or pharmaceutically acceptable salt or solvate thereof according to any one of embodiments 6 to 15 wherein X21 is D.

17. A dual agonist or pharmaceutically acceptable salt or solvate thereof according to embodiment 6 wherein X* is a peptide of formula III:

H[Aib]EG-X5-F-X7-SE-X10-ATILD-X16-X17-AA-X20-X21-FIAWLI-X28-X29-KITD (III)

wherein:

X5 is T or S;

X7 is T or S;

X10 is L or ψ;

X16 is G, E, A or ψ;

X17 is Q, E, K, L or ψ;

X20 is R or ψ;

X21 is D or L;

X28 is E, A or Q;

X29 is H, Y or Q;

and at least one of X5 and X7 is T.

18. A dual agonist or pharmaceutically acceptable salt or solvate thereof according to embodiment 6 wherein X* is a peptide of formula IV:

H[Aib]EG-X5-F-X7-SELATILD-X16-X17-AAR-X21-FIAWLI-X28-X29-KITD (IV)

wherein:

X5 is T or S;

X7 is T or S;

X16 is G or ψ;

X17 is E, K, L or ψ;

X21 is D or L;

X28 is E or A;

X29 is H, Y or Q;

and at least one of X5 and X7 is T.

19. A dual agonist or pharmaceutically acceptable salt or solvate thereof according to any one of embodiments 6 to 18 wherein X16 is 4⁴ and X17 is E, K or L.

20. A dual agonist or pharmaceutically acceptable salt or solvate thereof according to any one of embodiments 6 to 18 wherein X16 is G and X17 is ψ.

21. A dual agonist or pharmaceutically acceptable salt or solvate thereof according to embodiment 19 or embodiment 20 wherein:

X21 is D and X28 is E;

X21 is D and X28 is A;

X21 is L and X28 is E;

X21 is Land X28 is A. 22. A dual agonist or pharmaceutically acceptable salt or solvate thereof according to embodiment 6 wherein X* is a peptide of formula V:

H[Aib]EG-X5-F-X7-SELATI LD-4)-QAARDFIAWLI-X28-X29-KITD (V)

wherein

X5 is T or S;

X7 is T or S;

X28 is Q, E, A, H, Y, L, K, R or S;

X29 is H, Y or Q;

and at least one of X5 and X7 is T.

23. A dual agonist or pharmaceutically acceptable salt or solvate thereof according to any one of embodiments 6 to 22 wherein:

X5 is S and X7 is T;

X5 is T and X7 is S; or

X5 is T and X7 is T.

24. A dual agonist or pharmaceutically acceptable salt or solvate thereof according to embodiment 23 wherein X5 is S and X7 is T, or X5 is T and X7 is T.

25. A dual agonist or pharmaceutically acceptable salt or solvate thereof according to any one of embodiments 6 to 24 wherein 4⁴ is a Lys residue whose side chain is conjugated to the substituent Z¹— or Z¹—Z²—.

26. A dual agonist or pharmaceutically acceptable salt or solvate thereof according to any one of embodiments 6 to 25 wherein Z¹— is dodecanoyl, tetradecanoyl, hexadecanoyl, octadecanoyl or eicosanoyl.

27. A dual agonist or pharmaceutically acceptable salt or solvate thereof according to any one of embodiments 6 to 25 wherein Z¹— is:

13-carboxytridecanoyl, i.e. HOOC—(CH₂)₁₂—(CO)—;

15-carboxypentadecanoyl, i.e. HOOC—(CH₂)₁₄—(CO)—;

17-carboxyheptadecanoyl, i.e. HOOC—(CH₂)₁₆—(CO)—;

19-carboxynonadecanoyl, i.e. HOOC—(CH₂)₁₈—(CO)—; or

21-carboxyheneicosanoyl, i.e. HOOC—(CH₂)₂₀—(CO)—.

28. A dual agonist or pharmaceutically acceptable salt or solvate thereof according to any one of embodiments 6 to 27 wherein Z² is absent.

29. A dual agonist or pharmaceutically acceptable salt or solvate thereof according to any one of embodiments 6 to 27 wherein Z² comprises Z^{S1} alone or in combination with Z^{S2} and/or Z^{S3}.

30. A dual agonist or pharmaceutically acceptable salt or solvate thereof according to embodiment 29 wherein:

—Z^{S1}— is isoGlu, β-Ala, isoLys, or 4-aminobutanoyl;

—Z^{S2}—, when present, is -(Peg3)ₘ- where m is 1, 2, or 3; and

—Z^{S3}— is a peptide sequence of 1-6 amino acid units independently selected from the group consisting of A, L, S, T, Y, Q, D, E, K, k, R, H, F and G, such as the peptide sequence KEK.

31. A dual agonist or pharmaceutically acceptable salt or solvate thereof according to embodiment 29 or embodiment 30 wherein Z² has the formula —Z^{S1}—Z^{S3}—Z^{S2}—, where Z^{S1} is bonded to Z¹ and Z^{S2} is bonded to the side chain of the amino acid component of ψ.

32. A dual agonist or pharmaceutically acceptable salt or solvate thereof according to embodiment 31 wherein —Z²— is:

isoGlu(Peg3)₀₋₃;

β-Ala(Peg3)₀₋₃;

isoLys(Peg3)₀₋₃;

4-aminobutanoyl(Peg3)₀₋₃; or

isoGlu-KEK-(Peg3)₀₋₃.

33. A dual agonist or pharmaceutically acceptable salt or solvate thereof according to any one of embodiments 6 to 25 wherein Z¹— or Z¹—Z²— is:

[17-carboxy-heptadecanoyl]-isoGlu;

[17-Carboxy-heptadecanoyl]-isoGlu-KEK-Peg3-;

[17-carboxy-heptadecanoyl]-isoGlu-Peg3-;

[19-Carboxy-nonadecanoyl]-isoGlu-;

[19-Carboxy-nonadecanoyl]-isoGlu-KEK-;

[19-Carboxy-nonadecanoyl]-isoGlu-KEK-Peg3-;

[19-carboxy-nonadecanoyl]-isoGlu-KEK-Peg3-Peg3-;

[19-carboxy-nonadecanoyl]-isoGlu-Peg3-Peg3-;

[19-carboxy-nonadecanoyl]-isoLys-Peg3-Peg3-Peg3-;

[Hexadecanoyl]-βAla-;

[Hexadecanoyl]-isoGlu-; or

Octadecanoyl-.

34. A dual agonist or pharmaceutically acceptable salt or solvate thereof according to embodiment 33 wherein ψ is:

K([17-carboxy-heptadecanoyl]-isoGlu).

K([17-Carboxy-heptadecanoyl]-isoGlu-KEK-Peg3);

K([17-carboxy-heptadecanoyl]-isoGlu-Peg3);

K([19-Carboxy-nonadecanoyl]-isoGlu);

K([19-Carboxy-nonadecanoyl]-isoGlu-KEK);

K([19-Carboxy-nonadecanoyl]-isoGlu-KEK-Peg3);

K([19-carboxy-nonadecanoyl]-isoGlu-KEK-Peg3-Peg3);

K([19-carboxy-nonadecanoyl]-isoGlu-Peg3-Peg3);

K([19-carboxy-nonadecanoyl]-isoLys-Peg3-Peg3-Peg3);

K([Hexadecanoyl]-βAla-;

K([Hexadecanoyl]-isoGlu); or

K(Octadecanoyl).

35. A dual agonist or pharmaceutically acceptable salt or solvate thereof according to any one of embodiments 6 to 34 wherein U is 1-10 amino acids in length, 1-7 amino acids in length, 3-7 amino acids in length, 1-6 amino acids in length, or 3-6 amino acids in length.

36. A dual agonist or pharmaceutically acceptable salt or solvate thereof according to any one of embodiments 6 to 35 wherein U includes at least one charged amino acid , e.g. two or more charged amino acids.

37. A dual agonist or pharmaceutically acceptable salt or solvate thereof according to embodiment 36 wherein U includes at least 1 positively charged amino acid and at least one negatively charged amino acid.

38. A dual agonist or pharmaceutically acceptable salt or solvate thereof according to embodiment 8 or embodiment 37 wherein U is a chain of alternately positively and negatively charged amino acids.

39. A dual agonist or pharmaceutically acceptable salt or solvate thereof according to any one of embodiments 36 to 38 wherein U comprises residues selected only from K, k, E and ψ.

40. A dual agonist or pharmaceutically acceptable salt or solvate thereof according to embodiment 39 wherein U is K₃, K₄, K₅, K₆ , K₇, k₃, k₄, k₅, k₆ or k₇.

41. A dual agonist or pharmaceutically acceptable salt or solvate thereof according to any one of embodiments 37 to 38 wherein U is KEK, EKEKEK, EkEkEk, AKAAEK, AKEKEK or ATILEK.

42. A dual agonist or pharmaceutically acceptable salt or solvate thereof according to any one of embodiments 36 to 38 wherein U is K₁₋₁₄-ψ, K₁₋₉-ψ, K₁₋₆-ψ, k₁₋₁₄-ψ, k₁₋₉-ψ, k₁₋₆-ψ, KEψ, EKEKEψ, EkEkEψ AKAAEψ, AKEKEψ or ATI LEψ.

43. A dual agonist or pharmaceutically acceptable salt or solvate thereof according to any one of embodiments 6 to 34 wherein U is absent.

44. A dual agonist or pharmaceutically acceptable salt or solvate thereof according to any one of embodiments 6 to 43 wherein R¹ is Hy and/or R² is OH.

45. A dual agonist or pharmaceutically acceptable salt or solvate thereof according to any one of embodiments 6 to 44 wherein X* or X*-U has the sequence:

H[Aib]EGTFSSELATILDΨEAARDFIAWLIEHKITD;
H[Aib]EGSFTSELATILDΨEAARDFIAWLIEHKITD;
H[Aib]EGTFTSELATILDΨEAARDFIAWLIEHKITD;
H[AIb]EGTFSSELATILDΨKAARDFIAWLIEHKITD;
H[Aib]EGSFTSELATILDΨKAARDFIAWLIEHKITD;
H[Aib]EGTFTSELATILDΨKAARDFIAWLIEHKITD;
H[Aib]EGTFSSELATILDGΨAARDFIAWLIEHKITD;
H[Aib]EGSFTSELATILDGΨAARDFIAWLIEHKITD;
H[Aib]EGTFTSELATILDGΨAARDFIAWLIEHKITD;
H[Aib]EGTFSSELATILDΨLAARDFIAWLIEHKITD;
H[Aib]EGSFTSELATILDΨLAARDFIAWLIEHKITD;
H[Aib]EGTFTSELATILDΨLAARDFIAWLIEHKITD;
H[Aib]EGTFSSELATILDΨLAARDFIAWLIAHKITD;
H[Aib]EGSFTSELATILDΨLAARDFIAWLIAHKITD;
H[Aib]EGTFTSELATILDΨLAARDFIAWLIAHKITD;
H[Aib]EGTFTSELATILDΨEAARLFIAWLIEHKITD;
H[Aib]EGTFSSELATILDΨQAARDFIAWLIQHKITD;
H[Aib]EGSFTSELATILDΨQAARDFIAWLIQHKITD;
H[Aib]EGTFTSELATILDΨQAARDFIAWLIQHKITD;
H[Aib]EGTFSSELATILDΨQAARDFIAWLIEHKITD;
H[Aib]EGTFSSELATILDΨQAARDFIAWLIAHKITD;
H[Aib]EGSFTSELATILDΨQAARDFIAWLIAHKITD;
H[Aib]EGTFTSELATILDΨQAARDFIAWLIAHKITD;
H[Aib]EGSFTSELATILDΨQAARDFIAWLIEHKITD;
H[Aib]EGTFTSELATILDΨQAARDFIAWLIEHKITD;
H[Aib]EGSFTSELATILDΨQAARDFIAWLIHHKITD;
H[Aib]EGSFTSELATILDΨQAARDFIAWLIYHKITD;
H[Aib]EGSFTSELATILDΨQAARDFIAWLILHKITD;
H[Aib]EGSFTSELATILDΨQAARDFIAWLIKHKITD;
H[Aib]EGSFTSELATILDΨQAARDFIAWLIRHKITD;
H[Aib]EGSFTSELATILDΨQAARDFIAWLISHKITD
H[Aib]EGSFTSELATILDΨQAARDFIAWLQQHKITD;
H[Aib]EGSFTSELATILDΨQAARDFIAWLYQHKITD;
H[Aib]EGSFTSELATILDΨQAARDFIAWLKQHKITD;
H[Aib]EGSFTSELATILDΨQAARDFIAWLIQQKITD;
H[Aib]EGSFTSELATILDΨQAARDFIAWLIQYKITD;
H[Aib]EGTFSSELSTILEWQASREFIAWLIAYKITE;
H[Aib]EGTFSSELATILDEQAARDFIAWLIAHKITDkkkkkΨ;
H[Aib]EGTFTSELATILDEQAARDFIAWLIAHKITDkkkkkΨ;
H[Aib]EGSFTSELATILDEQAARDFIAWLIEHKITDkkkkkΨ;
H[Aib]EGSFTSEΨATILDEQAARDFIAWLIEHKITD;
H[Aib]EGSFTSELATILEGΨAARDFIAWLIEHKITD;
H[Aib]EGSFTSELATILDEQAAΨDFIAWLIEHKITD;
H[Aib]EGTFTSELATILDEQAAΨDFIAWLIEHKITD;
H[Aib]EGTFTSEΨATILDEQAARDFIAWLIEHKITD;
H[Aib]EGSFTSELATILDΨAARDFIAWLIEHKITD;
or
H[Aib]EGSFTSELATILDAKAAΨDFIAWLIEHKITD.

46. A dual agonist or pharmaceutically acceptable salt or solvate thereof according to embodiment 45 wherein X* or X*-U has the sequence:

H[Aib]EGTFSSELATILD[K*]EAARDFIAWLIEHKITD;
H[Aib]EGSFTSELATILD[K*]EAARDFIAWLIEHKITD;
H[Aib]EGTFTSELATILD[K*]EAARDFIAWLIEHKITD;
H[Aib]EGTFSSELATILD[K*]KAARDFIAWLIEHKITD;
H[Aib]EGSFTSELATILD[K*]KAARDFIAWLIEHKITD;
H[Aib]EGTFTSELATILD[K*]KAARDFIAWLIEHKITD;
H[Aib]EGTFSSELATILDG[K*]AARDFIAWLIEHKITD;
H[Aib]EGSFTSELATILDG[K*]AARDFIAWLIEHKITD;
H[Aib]EGTFTSELATILDG[K*]AARDFIAWLIEHKITD;
H[Aib]EGTFSSELATILD[K*]LAARDFIAWLIEHKITD;
H[Aib]EGSFTSELATILD[K*]LAARDFIAWLIEHKITD;
H[Aib]EGTFTSELATILD[K*]LAARDFIAWLIEHKITD;
H[Aib]EGTFSSELATILD[K*]LAARDFIAWLIAHKITD;
H[Aib]EGSFTSELATILD[K*]LAARDFIAWLIAHKITD;
H[Aib]EGTFTSELATILD[K*]LAARDFIAWLIAHKITD;
H[Aib]EGTFTSELATILD[K*]EAARLFIAWLIEHKITD;
H[Aib]EGTFSSELATILD[K*]QAARDFIAWLIQHKITD;
H[Aib]EGSFTSELATILD[K*]QAARDFIAWLIQHKITD;
H[Aib]EGTFTSELATILD[K*]QAARDFIAWLIQHKITD;
H[Aib]EGTFSSELATILD[K*]QAARDFIAWLIEHKITD;
H[Aib]EGTFSSELATILD[K*]QAARDFIAWLIAHKITD;
H[Aib]EGSFTSELATILD[K*]QAARDFIAWLIAHKITD;
H[Aib]EGTFTSELATILD[K*]QAARDFIAWLIAHKITD;
H[Aib]EGSFTSELATILD[K*]QAARDFIAWLIEHKITD;
H[Aib]EGTFTSELATILD[K*]QAARDFIAWLIEHKITD;
H[Aib]EGSFTSELATILD[K*]QAARDFIAWLIHHKITD;
H[Aib]EGSFTSELATILD[K*]QAARDFIAWLIYHKITD;
H[Aib]EGSFTSELATILD[K*]QAARDFIAWLILHKITD;
H[Aib]EGSFTSELATILD[K*]QAARDFIAWLIKHKITD;
H[Aib]EGSFTSELATILD[K*]QAARDFIAWLIRHKITD;
H[Aib]EGSFTSELATILD[K*]QAARDFIAWLISHKITD;
H[Aib]EGSFTSELATILD[K*]QAARDFIAWLQQHKITD;
H[Aib]EGSFTSELATILD[K*]QAARDFIAWLYQHKITD;
H[Aib]EGSFTSELATILD[K*]QAARDFIAWLKQHKITD;
H[Aib]EGSFTSELATILD[K*]QAARDFIAWLIQQKITD;
H[Aib]EGSFTSELATILD[K*]QAARDFIAWLIQYKITD;
H[Aib]EGTFSSELSTILE[K*]QASREFIAWLIAYKITE;
H[Aib]EGTFSSELATILDEQAARDFIAWLIAHKITDkkkkk[k*];
H[Aib]EGTFTSELATILDEQAARDFIAWLIAHKITDkkkkk[k*];
H[Aib]EGSFTSELATILDEQAARDFIAWLIEHKITDkkkkk[k*];
H[Aib]EGSFTSE[K*]ATILDEQAARDFIAWLIEHKITD;
H[Aib]EGSFTSELATILEG[K*]AARDFIAWLIEHKITD;
H[Aib]EGSFTSELATILDEQAA[K*]DFIAWLIEHKITD;
H[Aib]EGTFTSELATILDEQAA[K*]DFIAWLIEHKITD;
H[Aib]EGTFTSE[K*]ATILDEQAARDFIAWLIEHKITD;
H[Aib]EGSFTSELATILDA[K*]AARDFIAWLIEHKITD;
or
H[Aib]EGSFTSELATILDAKAA[K*]DFIAWLIEHKITD;

wherein K* or k* indicates an L or D lysine residue respectively in which the side chain is conjugated to the substituent Z¹— or Z¹Z²—.

47. A dual agonist or pharmaceutically acceptable salt or solvate thereof according to embodiment 46 wherein X* or X*-U has the sequence:

H[Aib]EGTFSSELATILD[K([17-carboxy-heptadecanoyl]-isoGlu)]EAARDFIAWLIEHKITD;
H[Aib]EGSFTSELATILD[K([17-carboxy-heptadecanoyl]-isoGlu)]EAARDFIAWLIEHKITD;
H[Aib]EGTFTSELATILD[K([17-carboxy-heptadecanoyl]-isoGlu)]EAARDFIAWLIEHKITD;
H[Aib]EGTFSSELATILD[K([17-carboxy-heptadecanoyl]-isoGlu)]KAARDFIAWLIEHKITD;
H[Aib]EGSFTSELATILD[K([17-carboxy-heptadecanoyl]-isoGlu)]KAARDFIAWLIEHKITD;
H[Aib]EGTFTSELATILD[K([17-carboxy-heptadecanoyl]-isoGlu)]KAARDFIAWLIEHKITD;
H[Aib]EGTFSSELATILDG[K([17-carboxy-heptadecanoyl]-isoGlu)]AARDFIAWLIEHKITD;
H[Aib]EGSFTSELATILDG[K([17-carboxy-heptadecanoyl]-isoGlu)]AARDFIAWLIEHKITD;
H[Aib]EGTFTSELATILDG[K([17-carboxy-heptadecanoyl]-isoGlu)]AARDFIAWLIEHKITD;
H[Aib]EGTFSSELATILD[K([17-carboxy-heptadecanoyl]-isoGlu)]LAARDFIAWLIEHKITD;
H[Aib]EGSFTSELATILD[K([17-carboxy-heptadecanoyl]-isoGlu)]LAARDFIAWLIEHKITD;
H[Aib]EGTFTSELATILD[K([17-carboxy-heptadecanoyl]-isoGlu)]LAARDFIAWLIEHKITD;
H[Aib]EGTFSSELATILD[K([17-carboxy-heptadecanoyl]-isoGlu)]LAARDFIAWLIAHKITD;
H[Aib]EGSFTSELATILD[K([17-carboxy-heptadecanoyl]-isoGlu)]LAARDFIAWLIAHKITD;
H[Aib]EGTFTSELATILD[K([17-carboxy-heptadecanoyl]-isoGlu)]LAARDFIAWLIAHKITD;
H[Aib]EGTFTSELATILD[K([17-carboxy-heptadecanoyl]-isoGlu)]EAARLFIAWLIEHKITD;
H[Aib]EGTFSSELATILD[K([17-carboxy-heptadecanoyl]-isoGlu)]QAARDFIAWLIQHKITD;
H[Aib]EGSFTSELATILD[K([17-carboxy-heptadecanoyl]-isoGlu)]QAARDFIAWLIQHKITD;
H[Aib]EGTFTSELATILD[K([17-carboxy-heptadecanoyl]-isoGlu)]QAARDFIAWLIQHKITD;
H[Aib]EGTFSSELATILD[K([17-carboxy-heptadecanoyl]-isoGlu)]QAARDFIAWLIEHKITD;
H[Aib]EGTFSSELATILD[K([17-carboxy-heptadecanoyl]-isoGlu)]QAARDFIAWLIAHKITD;
H[Aib]EGSFTSELATILD[K([17-carboxy-heptadecanoyl]-isoGlu)]QAARDFIAWLIAHKITD;
H[Aib]EGTFTSELATILD[K([17-carboxy-heptadecanoyl]-isoGlu)]QAARDFIAWLIAHKITD;
H[Aib]EGSFTSELATILD[K([17-carboxy-heptadecanoyl]-isoGlu)]QAARDFIAWLIEHKITD;
H[Aib]EGTFTSELATILD[K([17-carboxy-heptadecanoyl]-isoGlu)]QAARDFIAWLIEHKITD;
H[Aib]EGSFTSELATILD[K([17-carboxy-heptadecanoyl]-isoGlu)]QAARDFIAWLIHHKITD;
H[Aib]EGSFTSELATILD[K([17-carboxy-heptadecanoyl]-isoGlu)]QAARDFIAWLIYHKITD;
H[Aib]EGSFTSELATILD[K([17-carboxy-heptadecanoyl]-isoGlu)]QAARDFIAWLILHKITD;
H[Aib]EGSFTSELATILD[K([17-carboxy-heptadecanoyl]-isoGlu)]QAARDFIAWLIKHKITD;
H[Aib]EGSFTSELATILD[K([17-carboxy-heptadecanoyl]-isoGlu)]QAARDFIAWLIRHKITD;
H[Aib]EGSFTSELATILD[K([17-carboxy-heptadecanoyl]-isoGlu)]QAARDFIAWLISHKITD;
H[Aib]EGSFTSELATILD[K([Hexadecanoyl]-pAla)]QAARDFIAWLQQHKITD;
H[Aib]EGSFTSELATILD[K([17-carboxy-heptadecanoyl]iso-Glu-
Peg3)]QAARDFIAWLYQHKITD;
H[Aib]EGSFTSELATILD[K([19-carboxy-nonadecanoyl]iso-Glu-Peg3-
Peg3)]QAARDFIAWLKQHKITD;
H[Aib]EGSFTSELATILD[K([19-carboxy-nonadecanoyl]iso-Lys-Peg3-Peg3-
Peg3)]QAARDFIAWLIQQKITD;
H[Aib]EGSFTSELATILD[K(Octadecanoyl)]QAARDFIAWLIQYKITD;
H[Aib]EGTFSSELSTILE[K(Hexadecanoyl-isoGlu)]QASREFIAWLIAYKITE;
H[Aib]EGTFSSELATILDEQAARDFIAWLIAHKITDkkkkkk([17-carboxy-Heptadecanoyl]-
isoGlu)];
H[Aib]EGTFTSELATILDEQAARDFIAWLIAHKITDkkkkkk([17-carboxy-Heptadecanoyl]-
isoGlu)];
H[Aib]EGSFTSELATILDEQAARDFIAWLIEHKITDkkkkkk([17-carboxy-Heptadecanoyl]-
isoGlu)];
H[Aib]EGTFTSELATILD[K([19-Carboxy-nonadecanoyl]-isoGlu)]QAARDFIAWLIQHKITD;
H[Aib]EGSFTSE[K([19-carboxy-nonadecanoyl]iso-Glu-Peg3-
Peg3)]ATI LDEQAARDFIAWLIEHKITD;
H[Aib]EGSFTSELATILD[K([19-carboxy-nonadecanoyl]iso-Glu-Peg3-
Peg3)]KAARDFIAWLIEHKITD;
H[Aib]EGSFTSELATILEG[K([19-carboxy-nonadecanoyl]iso-Glu-Peg3-
Peg3)]AARDFIAWLIEHKITD;
H[Aib]EGSFTSELATILDEQAA[K([19-carboxy-nonadecanoyl]iso-Glu-Peg3-
Peg3)]DFIAWLIEHKITD;
H[Aib]EGTFTSELATILDEQAA[K([19-carboxy-nonadecanoyl]iso-Glu-Peg3-
Peg3)]DFIAWLIEHKITD;
H[Aib]EGTFSSELATILD[K([17-Carboxy-heptadecanoyl]-isoGlu-KEK-
Peg3)]QAARDFIAWLIQHKITD;
H[Aib]EGTFSSELATILD[K([19-Carboxy-nonadecanoyl]-isoGlu-KEK-
Peg3)]QAARDFIAWLIQHKITD;
H[Aib]EGTFSSELATILD[K([17-Carboxy-heptadecanoyl]-isoGlu-KEK-
Peg3)]QAARDFIAWLIEHKITD;
H[Aib]EGTFSSELATILD[K([19-Carboxy-nonadecanoyl]-isoGlu-KEK-
Peg3)]QAARDFIAWLIEHKITD;
H[Aib]EGTFTSELATILD[K([19-Carboxy-nonadecanoyl]-isoGlu-
KEK)]QAARDFIAWLIQHKITD;
H[Aib]EGTFTSELATILD[K([19-Carboxy-nonadecanoyl]-isoGlu-KEK-
Peg3)]QAARDFIAWLIQHKITD;
H[Aib]EGSFTSE[K([19-Carboxy-nonadecanoyl]-isoGlu-KEK-
Peg3)]ATILDEQAARDFIAWLIEHKITD;
H[Aib]EGTFTSE[K([19-Carboxy-nonadecanoyl]-isoGlu-KEK-
Peg3)]ATILDEQAARDFIAWLIEHKITD;
H[Aib]EGSFTSE[K([19-carboxy-nonadecanoyl]iso-Glu-KEK-Peg3-
Peg3)]ATILDEQAARDFIAWLIEHKITD;
H[Aib]EGTFTSELATILD[K([19-Carboxy-nonadecanoyl]-isoGlu-KEK-
Peg3)]QAARDFIAWLIEHKITD;
H[Aib]EGSFTSELATILD[K([19-Carboxy-nonadecanoyl]-isoGlu-KEK-
Peg3)]QAARDFIAWLIEHKITD;
H[Aib]EGSFTSELATILD[K([19-Carboxy-nonadecanoyl]-isoGlu-KEK-
Peg3)]QAARDFIAWLIAHKITD;
H[Aib]EGSFTSELATILD[K([19-Carboxy-nonadecanoyl]-isoGlu-KEK-
Peg3)]KAARDFIAWLIEHKITD;
H[Aib]EGSFTSELATILD[K([19-carboxy-nonadecanoyl]iso-Glu-KEK-Peg3-
Peg3)]QAARDFIAWLIEHKITD;
H[Aib]EGSFTSELATILEG[K([19-Carboxy-nonadecanoyl]-isoGlu-KEK-
Peg3)]AARDFIAWLIEHKITD;
H[Aib]EGSFTSELATILDA[K([19-Carboxy-nonadecanoyl]-isoGlu-KEK-
Peg3)]AARDFIAWLIEHKITD;
H[Aib]EGSFTSELATILDA[K([19-carboxy-nonadecanoyl]iso-Glu-KEK-Peg3-
Peg3)]AARDFIAWLIEHKITD;
H[Aib]EGSFTSELATILDEQAA[K([19-Carboxy-nonadecanoyl]-isoGlu-KEK-
Peg3)]DFIAWLIEHKITD;
H[Aib]EGTFTSELATILDEQAA[K([19-Carboxy-nonadecanoyl]-isoGlu-KEK-
Peg3)]DFIAWLIEHKITD;
H[Aib]EGSFTSELATILDEQAA[K([19-carboxy-nonadecanoyl]iso-Glu-KEK-Peg3-
Peg3)]DFIAWLIEHKITD;
H[Aib]EGTFTSELATILDEQAA[K([19-carboxy-nonadecanoyl]iso-Glu-KEK-Peg3-
Peg3)]DFIAWLIEHKITD;
or
H[Aib]EGSFTSELATILDAKAA[K([19-Carboxy-nonadecanoyl]-isoGlu-KEK-
Peg3)]DFIAWLIEHKITD.

48. A dual agonist or pharmaceutically acceptable salt or solvate thereof according to embodiment 47 which is:

Hy-H[Aib]EGTFSSELATILD[K([17-carboxy-heptadecanoyl]-
isoGlu)]EAARDFIAWLIEHKITD-OH (Compound 1);
Hy-H[Aib]EGSFTSELATILD[K([17-carboxy-heptadecanoyl]-
isoGlu)]EAARDFIAWLIEHKITD-OH (Compound 2);
Hy-H[Aib]EGTFTSELATILD[K([17-carboxy-heptadecanoyl]-
isoGlu)]EAARDFIAWLIEHKITD-OH (Compound 3);
Hy-H[Aib]EGTFSSELATILD[K([17-carboxy-heptadecanoyl]-
isoGlu)]KAARDFIAWLIEHKITD-OH (Compound 4);
Hy-H[Aib]EGSFTSELATILD[K([17-carboxy-heptadecanoyl]-
isoGlu)]KAARDFIAWLIEHKITD-OH (Compound 5);
Hy-H[Aib]EGTFTSELATILD[K([17-carboxy-heptadecanoyl]-
isoGlu)]KAARDFIAWLIEHKITD-OH (Compound 6);
Hy-H[Aib]EGTFSSELATILDG[K([17-carboxy-heptadecanoyl]-
isoGlu)]AARDFIAWLIEHKITD-OH (Compound 7);
Hy-H[Aib]EGSFTSELATILDG[K([17-carboxy-heptadecanoyl]-
isoGlu)]AARDFIAWLIEHKITD-OH (Compound 8);
Hy-H[Aib]EGTFTSELATILDG[K([17-carboxy-heptadecanoyl]-
isoGlu)]AARDFIAWLIEHKITD-OH (Compound 9);
Hy-H[Aib]EGTFSSELATILD[K([17-carboxy-heptadecanoyl]-
isoGlu)]LAARDFIAWLIEHKITD-OH (Compound 10);
Hy-H[Aib]EGSFTSELATILD[K([17-carboxy-heptadecanoyl]-
isoGlu)]LAARDFIAWLIEHKITD-OH (Compound 11);
Hy-H[Aib]EGTFTSELATILD[K([17-carboxy-heptadecanoyl]-
isoGlu)]LAARDFIAWLIEHKITD-OH (Compound 12);
Hy-H[Aib]EGTFSSELATILD[K([17-carboxy-heptadecanoyl]-
isoGlu)]LAARDFIAWLIAHKITD-OH (Compound 13);
Hy-H[Aib]EGSFTSELATILD[K([17-carboxy-heptadecanoyl]-
isoGlu)]LAARDFIAWLIAHKITD-OH (Compound 14);
Hy-H[Aib]EGTFTSELATILD[K([17-carboxy-heptadecanoyl]-
isoGlu)]LAARDFIAWLIAHKITD-OH (Compound 15);
Hy-H[Aib]EGTFTSELATILD[K([17-carboxy-heptadecanoyl]-
isoGlu)]EAARLFIAWLIEHKITD-OH (Compound 16);
Hy-H[Aib]EGTFSSELATILD[K([17-carboxy-heptadecanoyl]-
isoGlu)]QAARDFIAWLIQHKITD-OH (Compound 17);
Hy-H[Aib]EGSFTSELATILD[K([17-carboxy-heptadecanoyl]-
isoGlu)]QAARDFIAWLIQHKITD-OH (Compound 18);
Hy-H[Aib]EGTFTSELATILD[K([17-carboxy-heptadecanoyl]-
isoGlu)]QAARDFIAWLIQHKITD-OH (Compound 19);
Hy-H[Aib]EGTFSSELATILD[K([17-carboxy-heptadecanoyl]-
isoGlu)]QAARDFIAWLIEHKITD-OH (Compound 20);
Hy-H[Aib]EGTFSSELATILD[K([17-carboxy-heptadecanoyl]-
isoGlu)]QAARDFIAWLIAHKITD-OH (Compound 21);
Hy-H[Aib]EGSFTSELATILD[K([17-carboxy-heptadecanoyl]-
isoGlu)]QAARDFIAWLIAHKITD-OH (Compound 22);
Hy-H[Aib]EGTFTSELATILD[K([17-carboxy-heptadecanoyl]-
isoGlu)]QAARDFIAWLIAHKITD-OH (Compound 23);
Hy-H[Aib]EGSFTSELATILD[K([17-carboxy-heptadecanoyl]-
isoGlu)]QAARDFIAWLIEHKITD-OH (Compound 24);
Hy-H[Aib]EGTFTSELATILD[K([17-carboxy-heptadecanoyl]-
isoGlu)]QAARDFIAWLIEHKITD-OH (Compound 25);
Hy-H[Aib]EGSFTSELATILD[K([17-carboxy-heptadecanoyl]-
isoGlu)]QAARDFIAWLIHHKITD-OH (Compound 26);
Hy-H[Aib]EGSFTSELATILD[K([17-carboxy-heptadecanoyl]-
isoGlu)]QAARDFIAWLIYHKITD-OH (Compound 27);
Hy-H[Aib]EGSFTSELATILD[K([17-carboxy-heptadecanoyl]-
isoGlu)]QAARDFIAWLILHKITD-OH (Compound 28);
Hy-H[Aib]EGSFTSELATILD[K([17-carboxy-heptadecanoyl]-
isoGlu)]QAARDFIAWLIKHKITD-OH (Compound 29);
Hy-H[Aib]EGSFTSELATILD[K([17-carboxy-heptadecanoyl]-
isoGlu)]QAARDFIAWLIRHKITD-OH (Compound 30);
Hy-H[Aib]EGSFTSELATILD[K([17-carboxy-heptadecanoyl]-
isoGlu)]QAARDFIAWLISHKITD-OH (Compound 31).
Hy-H[Aib]EGSFTSELATILD[K([Hexadecanoyl]-pAla)]QAARDFIAWLQQHKITD-OH
(Compound 32);
Hy-H[Aib]EGSFTSELATILD[K([17-carboxy-heptadecanoyl]iso-Glu-
Peg3)]QAARDFIAWLYQHKITD-OH (Compound 33);
Hy-H[Aib]EGSFTSELATILD[K([19-carboxy-nonadecanoyl]iso-Glu-Peg3-
Peg3)]QAARDFIAWLKQHKITD-OH (Compound 34);
Hy-H[Aib]EGSFTSELATILD[K([19-carboxy-nonadecanoyl]iso-Lys-Peg3-Peg3-
Peg3)]QAARDFIAWLIQQKITD-OH (Compound 35);
Hy-H[Aib]EGSFTSELATILD[K(Octadecanoyl)]QAARDFIAWLIQYKITD-OH
(Compound 36);
Hy-H[Aib]EGTFSSELSTILE[K(Hexadecanoyl-isoGlu)]QASREFIAWLIAYKITE-OH
(Compound 37);
Hy-H[Aib]EGTFSSELATILDEQAARDFIAWLIAHKITDkkkkkk([17-carboxy-
Heptadecanoyl]-isoGlu)]-[NH2] (Compound 38);
Hy-H[Aib]EGTFTSELATILDEQAARDFIAWLIAHKITDkkkkkk([17-carboxy-
Heptadecanoyl]-isoGlu)]-[NH2] (Compound 39);
Hy-H[Aib]EGSFTSELATILDEQAARDFIAWLIEHKITDkkkkkk([17-carboxy-
Heptadecanoyl]-isoGlu)]-[NH2] (Compound 40);
Hy-H[Aib]EGTFTSELATILD[K([19-Carboxy-nonadecanoyl]-
isoGlu)]QAARDFIAWLIQHKITD-OH (Compound 41);
Hy-H[Aib]EGSFTSE[K([19-carboxy-nonadecanoyl]iso-Glu-Peg3-
Peg3)]ATILDEQAARDFIAWLIEHKITD-OH (Compound 42);
Hy-H[Aib]EGSFTSELATILD[K([19-carboxy-nonadecanoyl]iso-Glu-Peg3-
Peg3)]KAARDFIAWLIEHKITD-OH (Compound 43);
Hy-H[Aib]EGSFTSELATILEG[K([19-carboxy-nonadecanoyl]iso-Glu-Peg3-
Peg3)]AARDFIAWLIEHKITD-OH (Compound 44);
Hy-H[Aib]EGSFTSELATILDEQAA[K([19-carboxy-nonadecanoyl]iso-Glu-Peg3-
Peg3)]DFIAWLIEHKITD-OH (Compound 45);
Hy-H[Aib]EGTFTSELATILDEQAA[K([19-carboxy-nonadecanoyl]iso-Glu-Peg3-
Peg3)]DFIAWLIEHKITD-OH (Compound 46).
Hy-H[Aib]EGTFSSELATILD[K([17-Carboxy-heptadecanoyl]-isoGlu-KEK-
Peg3)]QAARDFIAWLIQHKITD-OH (Compound 47);
Hy-H[Aib]EGTFSSELATILD[K([19-Carboxy-nonadecanoyl]-isoGlu-KEK-
Peg3)]QAARDFIAWLIQHKITD-OH (Compound 48);
Hy-H[Aib]EGTFSSELATILD[K([17-Carboxy-heptadecanoyl]-isoGlu-KEK-
Peg3)]QAARDFIAWLIEHKITD-OH (Compound 49);
Hy-H[Aib]EGTFSSELATILD[K([19-Carboxy-nonadecanoyl]-isoGlu-KEK-
Peg3)]QAARDFIAWLIEHKITD-OH (Compound 50);
Hy-H[Aib]EGTFTSELATILD[K([19-Carboxy-nonadecanoyl]-isoGlu-
KEK)]QAARDFIAWLIQHKITD-OH (Compound 51);
Hy-H[Aib]EGTFTSELATILD[K([19-Carboxy-nonadecanoyl]-isoGlu-KEK-
Peg3)]QAARDFIAWLIQHKITD-OH (Compound 52);
Hy-H[Aib]EGSFTSE[K([19-Carboxy-nonadecanoyl]-isoGlu-KEK-
Peg3)]ATILDEQAARDFIAWLIEHKITD-OH (Compound 53);
Hy-H[Aib]EGTFTSE[K([19-Carboxy-nonadecanoyl]-isoGlu-KEK-
Peg3)]ATILDEQAARDFIAWLIEHKITD-OH (Compound 54);
Hy-H[Aib]EGSFTSE[K([19-carboxy-nonadecanoyl]iso-Glu-KEK-Peg3-
Peg3)]ATILDEQAARDFIAWLIEHKITD-OH (Compound 55);
Hy-H[Aib]EGTFTSELATILD[K([19-Carboxy-nonadecanoyl]-isoGlu-KEK-
Peg3)]QAARDFIAWLIEHKITD-OH (Compound 56);
Hy-H[Aib]EGSFTSELATILD[K([19-Carboxy-nonadecanoyl]-isoGlu-KEK-
Peg3)]QAARDFIAWLIEHKITD-OH (Compound 57);
Hy-H[Aib]EGSFTSELATILD[K([19-Carboxy-nonadecanoyl]-isoGlu-KEK-
Peg3)]QAARDFIAWLIAHKITD-OH (Compound 58);
Hy-H[Aib]EGSFTSELATILD[K([19-Carboxy-nonadecanoyl]-isoGlu-KEK-
Peg3)]KAARDFIAWLIEHKITD-OH (Compound 59);
Hy-H[Aib]EGSFTSELATILD[K([19-carboxy-nonadecanoyl]iso-Glu-KEK-Peg3-
Peg3)]QAARDFIAWLIEHKITD-OH (Compound 60);
Hy-H[Aib]EGSFTSELATILEG[K([19-Carboxy-nonadecanoyl]-isoGlu-KEK-
Peg3)]AARDFIAWLIEHKITD-OH (Compound 61);
Hy-H[Aib]EGSFTSELATILDA[K([19-Carboxy-nonadecanoyl]-isoGlu-KEK-
Peg3)]AARDFIAWLIEHKITD-OH (Compound 62);
Hy-H[Aib]EGSFTSELATILDA[K([19-carboxy-nonadecanoyl]iso-Glu-KEK-Peg3-
Peg3)]AARDFIAWLIEHKITD-OH (Compound 63);
Hy-H[Aib]EGSFTSELATILDEQAA[K([19-Carboxy-nonadecanoyl]-isoGlu-KEK-
Peg3)]DFIAWLIEHKITD-OH (Compound 64);
Hy-H[Aib]EGTFTSELATILDEQAA[K([19-Carboxy-nonadecanoyl]-isoGlu-KEK-
Peg3)]DFIAWLIEHKITD-OH (Compound 65);
Hy-H[Aib]EGSFTSELATILDEQAA[K([19-carboxy-nonadecanoyl]iso-Glu-KEK-Peg3-
Peg3)]DFIAWLIEHKITD-OH (Compound 66);
Hy-H[Aib]EGTFTSELATILDEQAA[K([19-carboxy-nonadecanoyl]iso-Glu-KEK-Peg3-
Peg3)]DFIAWLIEHKITD-OH (Compound 67);
or
Hy-H[Aib]EGSFTSELATILDAKAA[K([19-Carboxy-nonadecanoyl]-isoGlu-KEK-
Peg3)]DFIAWLIEHKITD-OH (Compound 68).

49. The composition for use according to any one of embodiments 1 to 5 wherein said GLP-1/GLP-2 dual agonist is a compound represented by the formula:

R¹—X*-U—R²

wherein:

R¹ is hydrogen (Hy), C₁₋₄ alkyl (e.g. methyl), acetyl, formyl, benzoyl or trifluoroacetyl;

R² is NH₂ or OH;

X* is a peptide of formula I:

H-X2-EG-X5-F-X7-X8-E-X10-X11-TIL-X15-X16-X17-A-X19-X20-X21-FI-X24-WL-X27-X28-X29-KIT-X33 (I)

wherein

X2 is Aib or G;

X5 is S or T;

X7 is S or T;

X8 is S, E or D;

X10 is L, M or V;

X11 is A, N or S;

X15 is D or E

X16 is E, A or G;

X17 is Q, E, L or K;

X19 is A, V or S;

X20 is R or K;

X21 is D, L or E;

X24 is A, Nor S;

X27 is I, Y, Q, H or K;

X28 is A, E, H, Y, L, K, Q, R or S;

X29 is H, Y, K or Q;

X33 is D or E;

U is absent or a sequence of 1-15 residues, each independently selected from K and k; and wherein at least one of X5 and X7 is T;

or a pharmaceutically acceptable salt or solvate thereof.

50. The composition for use according to any preceding embodiment, wherein said dual agonist is in admixture with a carrier.

51. The composition for use according to embodiment 50 wherein the composition is a pharmaceutical composition and the carrier is a pharmaceutically acceptable carrier.

52 The pharmaceutical composition for use according to embodiment 51 wherein said dual agonist is in admixture with a pharmaceutically acceptable carrier, excipient or vehicle.

53. The composition for use according to any preceding embodiment wherein said GLP-1/GLP-2 dual agonist is administered at a dose of about 0.1 pmol/kg to 500 μmol/kg body weight, preferably about 50 pmol/kg to 500 nmol/kg.

54. The composition for use according to any preceding embodiment which is administered in an amount effective to achieve a blood concentration of at least 0.1 nmol/L of said dual agonist in said patient.

55. The composition for use according to any preceding embodiment, which is effective to reduce the amount of parenteral nutrition received by the patient.

56. The composition for use according to any preceding embodiment, which is effective to eliminate a need for the patient to receive parenteral nutrition or facilitate the patient's return to enteral feeding.

57. The composition for use according to any preceding embodiment wherein said patient is receiving total parenteral nutrition.

58. The composition for use according to any preceding embodiment, wherein said patient may receive increased enteral nutrition with the composition compared to without the composition.

59. The composition for use according to any preceding embodiment, wherein the composition is effective to improve intestinal mucosal properties.

1. A composition comprising a GLP-1/GLP-2 dual agonist for use in the treatment of a patient receiving parenteral nutrition.

2. The composition for use according to claim 1 for use in prophylaxis or treatment of malabsorption, ulcers, short-bowel syndrome, cul-de-sac syndrome, inflammatory bowel disease, irritable bowel syndrome, pouchitis, celiac sprue, tropical sprue, hypogammaglobulinemic sprue, mucositis induced by chemotherapy or radiation therapy, diarrhea induced by chemotherapy or radiation therapy, low grade inflammation, metabolic endotoxemia, necrotising enterocolitis, primary biliary cirrhosis, hepatitis, fatty liver disease, or gastrointestinal side-effects of inflammatory conditions.

3. The composition for use according to claim 1 or claim 2 wherein said patient has intestinal insufficiency or failure.

4. The composition for use according to any preceding claim wherein said patient has hepatic impairment or insufficiency.

5. The composition for use according to any preceding claim wherein said GLP-1/GLP-2 dual agonist is a peptide.

6. The composition for use according to any preceding claim wherein said GLP-1/GLP-2 dual agonist is a compound represented by the formula:
R¹—X*-U—R²
wherein:
R¹ is hydrogen (Hy), C₁₋₄ alkyl (e.g. methyl), acetyl, formyl, benzoyl or trifluoroacetyl;
R² is NH₂ or OH;
X* is a peptide of formula I:
H-X2-EG-X5-F-X7-X8-E-X10-X11-TIL-X15-X16-X17-A-X19-X20-X21-FI-X24-WL-X27-X28-X29-KIT-X33 (I)
wherein:
X2 is Aib or G
X5 is T or S;
X7 is T or S;
X8 is S, E or D;
X10 is L, M, V or ψ;
X11 is A, N or S;
X15 is D or E;
X16 is G, E, A or ψ;
X17 is Q, E, K, L or ψ;
X19 is A, V or S;
X20 is R, K or ψ;
X21 is D, L or E;
X24 is A, Nor S;
X27 is I, Q, K, H or Y;
X28 is Q, E, A, H, Y, L, K, R or S;
X29 is H, Y, K or Q;
X33 is D or E;
U is absent or a sequence of 1-15 residues each independently selected from K, k, E, A, T, I, L and ψ;
the molecule contains one and only one ψ, wherein ψ is a residue of K, k, R, Orn, Dap or Dab in which the side chain is conjugated to a substituent having the formula Z¹— or Z¹—Z²—, wherein
Z¹— is CH₃—(CH₂)₁₀₋₂₂—(CO)— or HOOC—(CH₂)₁₀₋₂₂—(CO)—; and
—Z²— is selected from —Z^{S1}—, —Z^{S1}—Z^{S2}—, —Z^{S2}—Z^{S1}—Z^{S2}—, —Z^{S3}—, —Z^{S1}Z^{S3}—, —Z^{S2}Z^{S3}—, —Z^{S3}Z^{S1}—, —Z^{S3}Z^{S2}—, —Z^{S1}Z^{S2}Z^{S3}—, —Z^{S1}Z^{S3}Z^{S2}—, —Z^{S2}Z^{S1}Z^{S3}—, —Z^{S2}Z^{S3}Z^{S1}—, —Z^{S3}Z^{S1}Z^{S2}—, —Z^{S3}Z^{S2}Z^{S1}—, Z^{S2}Z^{S3}Z^{S2}—
wherein
Z^{S1} is isoGlu, β-Ala, isoLys, or 4-aminobutanoyl;
Z^{S2} is _{—} (Peg3),- where m is 1, 2, or 3; and
—Z^{S3}— is a peptide sequence of 1-6 amino acid units independently selected from the group consisting of A, L, S, T, Y, Q, D, E, K, k, R, H, F and G;
and wherein at least one of X5 and X7 is T;
or a pharmaceutically acceptable salt or solvate thereof.

7. The composition for use according to any preceding claim, wherein said dual agonist is in admixture with a carrier.

8. The composition for use according to claim 7 wherein the composition is a pharmaceutical composition and the carrier is a pharmaceutically acceptable carrier.

9. The pharmaceutical composition for use according to claim 8 wherein said dual agonist is in admixture with a pharmaceutically acceptable carrier, excipient or vehicle.

10. The composition for use according to any preceding claim wherein said GLP-1/GLP-2 dual agonist is administered at a dose of about 0.1 pmol/kg to 500 μmol/kg body weight, preferably about 50 pmol/kg to 500 nmol/kg.

11. The composition for use according to any preceding claim which is administered in an amount effective to achieve a blood concentration of at least 0.1 nmol/L of said dual agonist in said patient.

12. The composition for use according to any preceding claim, which is effective to reduce the amount of parenteral nutrition received by the patient.

13. The composition for use according to any preceding claim wherein said patient is receiving total parenteral nutrition.

14. The composition for use according to any preceding claim, wherein said patient may receive increased enteral nutrition with the composition compared to without the composition.

15. The composition for use according to any preceding claim, wherein the composition is effective to improve intestinal mucosal properties.


SEQUENCE LISTING (187 sequences)

SEQ ID NO: 1 - protein, 33 aa, Artificial Sequence
  GLP-1/GLP-2 dual agonist peptide
  MISC_FEATURE (2)..(2): Xaa is Aib or G
  MISC_FEATURE (5)..(5): Xaa is T or S
  MISC_FEATURE (7)..(7): Xaa is T or S
  MISC_FEATURE (8)..(8): Xaa is S, E or D
  MISC_FEATURE (10)..(10): Xaa is L, M, V or a residue of K, k (D-lys), R, Orn, Dap or Dab in which the side chain is conjugated to a substituent
  MISC_FEATURE (11)..(11): Xaa is A, N or S
  MISC_FEATURE (15)..(15): Xaa is D or E
  MISC_FEATURE (16)..(16): Xaa is G, E, A or a residue of K, k (D-lys), R, Orn, Dap or Dab in which the side chain is conjugated to a substituent
  MISC_FEATURE (17)..(17): Xaa is Q, E, K, L or a residue of K, k (D-lys), R, Orn, Dap or Dab in which the side chain is conjugated to a substituent
  MISC_FEATURE (19)..(19): Xaa is A, V or S
  MISC_FEATURE (20)..(20): Xaa is R, K or a residue of K, k (D-lys), R, Orn, Dap or Dab in which the side chain is conjugated to a substituent
  MISC_FEATURE (21)..(21): Xaa is D, L or E
  MISC_FEATURE (24)..(24): Xaa is A, N or  S
  MISC_FEATURE (27)..(27): Xaa is I, Q, K, H or Y
  MISC_FEATURE (28)..(28): Xaa is Q, E, A, H, Y, L, K, R or S
  MISC_FEATURE (29)..(29): Xaa is H, Y, K or Q
  MISC_FEATURE (33)..(33): Xaa is D or E
HXEGXFXXEXXTILXXXAXXXFIXWLXXXKITX

SEQ ID NO: 2 - protein, 33 aa, Artificial Sequence
  GLP-1/GLP-2 dual agonist peptide
  MISC_FEATURE (2)..(2): Xaa is Aib or G
  MISC_FEATURE (5)..(5): Xaa is T or S
  MISC_FEATURE (7)..(7): Xaa is T or S
  MISC_FEATURE (10)..(10): Xaa is L or a residue of K, k (D-lys), R, Orn, Dap or Dab in which the side chain is conjugated to a substituent
  MISC_FEATURE (11)..(11): Xaa is A or S
  MISC_FEATURE (15)..(15): Xaa is D or E
  MISC_FEATURE (16)..(16): Xaa is G, E, A or a residue of K, k (D-lys), R, Orn, Dap or Dab in which the side chain is conjugated to a substituent
  MISC_FEATURE (17)..(17): Xaa is Q, E, K, L or a residue of K, k (D-lys), R, Orn, Dap or Dab in which the side chain is conjugated to a substituent
  MISC_FEATURE (19)..(19): Xaa is A or S
  MISC_FEATURE (20)..(20): Xaa is R or a residue of K, k (D-lys), R, Orn, Dap or Dab in which the side chain is conjugated to a substituent
  MISC_FEATURE (21)..(21): Xaa is D, L or E
  MISC_FEATURE (27)..(27): Xaa is I, Q, K or Y
  MISC_FEATURE (28)..(28): Xaa is Q, E, A, H, Y, L, K, R or S
  MISC_FEATURE (29)..(29): Xaa is H, Y or Q
  MISC_FEATURE (33)..(33): Xaa is D or E
HXEGXFXSEXXTILXXXAXXXFIAWLXXXKITX

SEQ ID NO: 3 - protein, 33 aa, Artificial Sequence
  GLP-1/GLP-2 Dual agonist peptide
  MISC_FEATURE (2)..(2): Xaa is Aib or G
  MISC_FEATURE (5)..(5): Xaa is T or S
  MISC_FEATURE (7)..(7): Xaa is T or S
  MISC_FEATURE (16)..(16): Xaa is G or a residue of K, k (D-lys), R, Orn, Dap or Dab in which the side chain is conjugated to a substituent
  MISC_FEATURE (17)..(17): Xaa is Q, E, K, L or a residue of K, k (D-lys), R, Orn, Dap or Dab in which the side chain is conjugated to a substituent
  MISC_FEATURE (21)..(21): Xaa is D or L
  MISC_FEATURE (28)..(28): Xaa is Q, E, A, H, Y, L, K, R or S
  MISC_FEATURE (29)..(29): Xaa is H, Y or Q
HXEGXFXSELATILDXXAARXFIAWLIXXKITD

SEQ ID NO: 4 - protein, 33 aa, Artificial Sequence
  GLP-1/GLP-2 dual agonist peptide
  MOD_RES (2)..(2): Aib
  MISC_FEATURE (5)..(5): Xaa is T or S where at least one of Xaa at position 5 or 7 is T
  MISC_FEATURE (7)..(7): Xaa is T or S where at least one of Xaa at position 5 or 7 is T
  MISC_FEATURE (10)..(10): Xaa is L or a residue of K, k (D-lys), R, Orn, Dap or Dab in which the side chain is conjugated to a substituent
  MISC_FEATURE (16)..(16): Xaa is G, E, A or a residue of K, k (D-lys), R, Orn, Dap or Dab in which the side chain is conjugated to a substituent
  MISC_FEATURE (17)..(17): Xaa is Q, E, K, L or a residue of K, k (D-lys), R, Orn, Dap or Dab in which the side chain is conjugated to a substituent
  MISC_FEATURE (20)..(20): Xaa is R or a residue of K, k (D-lys), R, Orn, Dap or Dab in which the side chain is conjugated to a substituent
  MISC_FEATURE (21)..(21): Xaa is D or L
  MISC_FEATURE (28)..(28): Xaa is E, A or Q
  MISC_FEATURE (29)..(29): Xaa is H, Y or Q
HXEGXFXSEXATILDXXAAXXFIAWLIXXKITD

SEQ ID NO: 5 - protein, 33 aa, Artificial Sequence; identical to SEQ ID NO: 3
  GLP-1/GLP-2 Dual agonist peptide
  MOD_RES (2)..(2): Aib
  MISC_FEATURE (5)..(5): Xaa is T or S where at least one of Xaa at position 5 or 7 is T
  MISC_FEATURE (7)..(7): Xaa is T or S where at least one of Xaa at position 5 or 7 is T
  MISC_FEATURE (16)..(16): Xaa is G or a residue of K, k (D-lys), R, Orn, Dap or Dab in which the side chain is conjugated to a substituent
  MISC_FEATURE (17)..(17): Xaa is E, K, L or a residue of K, k (D-lys), R, Orn, Dap or Dab in which the side chain is conjugated to a substituent
  MISC_FEATURE (21)..(21): Xaa is D or L
  MISC_FEATURE (28)..(28): Xaa is E or A
  MISC_FEATURE (29)..(29): Xaa is H, Y or Q

SEQ ID NO: 6 - protein, 33 aa, Artificial Sequence
  GLP-1/GLP-2 Dual agonist peptide
  MOD_RES (2)..(2): Aib
  MISC_FEATURE (5)..(5): Xaa is T or S where at least one of Xaa at position 5 or 7 is T
  MISC_FEATURE (7)..(7): Xaa is T or S where at least one of Xaa at position 5 or 7 is T
  MISC_FEATURE (16)..(16): Xaa is a residue of K, k (D-lys), R, Orn, Dap or Dab in which the side chain is conjugated to a substituent
  MISC_FEATURE (28)..(28): Xaa is Q, E, A, H, Y, L, K, R or S, e.g. Q, E, A, H, Y or L
  MISC_FEATURE (29)..(29): Xaa is H, Y or Q
HXEGXFXSELATILDXQAARDFIAWLIXXKITD

SEQ ID NO: 7 - protein, 6 aa, Artificial Sequence
  GLP-1/GLP-2 Dual agonist peptide
  MISC_FEATURE (1)..(6): Lys at positions 1-6 is D-Lys
KKKKKK

SEQ ID NO: 8 - protein, 4 aa, Artificial Sequence
  GLP-1/GLP-2 Dual agonist peptide
  MOD_RES (1)..(1): Xaa is isoGlu
XKEK

SEQ ID NO: 9 - protein, 5 aa, Artificial Sequence
  GLP-1/GLP-2 Dual agonist peptide
  MISC_FEATURE (2)..(2): Xaa is [17-Carboxy-heptadecanoyl]-isoGlu
KXKEK

SEQ ID NO: 10 - protein, 5 aa, Artificial Sequence
  GLP-1/GLP-2 Dual agonist peptide
  MISC_FEATURE (2)..(2): Xaa is [19-Carboxy-nonadecanoyl]-isoGlu
KXKEK

SEQ ID NO: 11 - protein, 6 aa, Artificial Sequence
  GLP-1/GLP-2 Dual agonist peptide
EKEKEK

SEQ ID NO: 12 - protein, 6 aa, Artificial Sequence
  GLP-1/GLP-2 Dual agonist peptide
  MISC_FEATURE (2)..(2): Xaa is D-lys
  MISC_FEATURE (4)..(4): Xaa is D-lys
  MISC_FEATURE (6)..(6): Xaa is D-lys
EXEXEX

SEQ ID NO: 13 - protein, 6 aa, Artificial Sequence
  GLP-1/GLP-2 Dual agonist peptide
AKAAEK

SEQ ID NO: 14 - protein, 6 aa, Artificial Sequence
  GLP-1/GLP-2 Dual agonist peptide
AKEKEK

SEQ ID NO: 15 - protein, 6 aa, Artificial Sequence
  GLP-1/GLP-2 Dual agonist peptide
ATILEK

SEQ ID NO: 16 - protein, 6 aa, Artificial Sequence
  GLP-1/GLP-2 Dual agonist peptide
  MISC_FEATURE (6)..(6): Xaa is a residue of K, k (D-lys), R, Orn, Dap or Dab in which the side chain is conjugated to a substituent
EKEKEX

SEQ ID NO: 17 - protein, 6 aa, Artificial Sequence
  GLP-1/GLP-2 Dual agonist peptide
  MISC_FEATURE (2)..(2): Xaa is D-lys
  MISC_FEATURE (4)..(4): Xaa is D-lys
  MISC_FEATURE (6)..(6): Xaa is a residue of K, k (D-lys), R, Orn, Dap or Dab in which the side chain is conjugated to a substituent
EXEXEX

SEQ ID NO: 18 - protein, 6 aa, Artificial Sequence
  GLP-1/GLP-2 Dual agonist peptide
  MISC_FEATURE (6)..(6): Xaa is a residue of K, k (D-lys), R, Orn, Dap or Dab in which the side chain is conjugated to a substituent
AKAAEX

SEQ ID NO: 19 - protein, 6 aa, Artificial Sequence
  GLP-1/GLP-2 Dual agonist peptide
  MISC_FEATURE (6)..(6): Xaa is a residue of K, k (D-lys), R, Orn, Dap or Dab in which the side chain is conjugated to a substituent
AKEKEX

SEQ ID NO: 20 - protein, 6 aa, Artificial Sequence
  GLP-1/GLP-2 Dual agonist peptide
  MISC_FEATURE (6)..(6): xaa is a residue of K, k (D-lys), R, Orn, Dap or Dab in which the side chain is conjugated to a substituent
ATILEX

SEQ ID NO: 21 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFSSELATILDXEAARDFIAWLIEHKITD

SEQ ID NO: 22 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXEAARDFIAWLIEHKITD

SEQ ID NO: 23 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFTSELATILDXEAARDFIAWLIEHKITD

SEQ ID NO: 24 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFSSELATILDXKAARDFIAWLIEHKITD

SEQ ID NO: 25 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXKAARDFIAWLIEHKITD

SEQ ID NO: 26 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFTSELATILDXKAARDFIAWLIEHKITD

SEQ ID NO: 27 - protein, 33 aa, Artificial Sequence
  GLP-1/GLP-2 Dual agonist peptide
  MOD_RES (2)..(2): Aib
  MISC_FEATURE (17)..(17): Xaa is a residue of K, k (D-lys), R, Orn, Dap or Dab in which the side chain is conjugated to a substituent
HXEGTFSSELATILDGXAARDFIAWLIEHKITD

SEQ ID NO: 28 - protein, 33 aa, Artificial Sequence
  GLP-1/GLP-2 Dual agonist peptide
  MOD_RES (2)..(2): Aib
  MISC_FEATURE (17)..(17): Xaa is a residue of K, k (D-lys), R, Orn, Dap or Dab in which the side chain is conjugated to a substituent
HXEGSFTSELATILDGXAARDFIAWLIEHKITD

SEQ ID NO: 29 - protein, 33 aa, Artificial Sequence
  GLP-1/GLP-2 Dual agonist peptide
  MOD_RES (2)..(2): Aib
  MISC_FEATURE (17)..(17): Xaa is a residue of K, k (D-lys), R, Orn, Dap or Dab in which the side chain is conjugated to a substituent
HXEGTFTSELATILDGXAARDFIAWLIEHKITD

SEQ ID NO: 30 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFSSELATILDXLAARDFIAWLIEHKITD

SEQ ID NO: 31 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXLAARDFIAWLIEHKITD

SEQ ID NO: 32 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFTSELATILDXLAARDFIAWLIEHKITD

SEQ ID NO: 33 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFSSELATILDXLAARDFIAWLIAHKITD

SEQ ID NO: 34 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXLAARDFIAWLIAHKITD

SEQ ID NO: 35 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFTSELATILDXLAARDFIAWLIAHKITD

SEQ ID NO: 36 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFTSELATILDXEAARLFIAWLIEHKITD

SEQ ID NO: 37 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFSSELATILDXQAARDFIAWLIQHKITD

SEQ ID NO: 38 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLIQHKITD

SEQ ID NO: 39 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFTSELATILDXQAARDFIAWLIQHKITD

SEQ ID NO: 40 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFSSELATILDXQAARDFIAWLIEHKITD

SEQ ID NO: 41 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFSSELATILDXQAARDFIAWLIAHKITD

SEQ ID NO: 42 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLIAHKITD

SEQ ID NO: 43 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFTSELATILDXQAARDFIAWLIAHKITD

SEQ ID NO: 44 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLIEHKITD

SEQ ID NO: 45 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFTSELATILDXQAARDFIAWLIEHKITD

SEQ ID NO: 46 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLIHHKITD

SEQ ID NO: 47 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLIYHKITD

SEQ ID NO: 48 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLILHKITD

SEQ ID NO: 49 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLIKHKITD

SEQ ID NO: 50 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLIRHKITD

SEQ ID NO: 51 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLISHKITD

SEQ ID NO: 52 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLQQHKITD

SEQ ID NO: 53 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLYQHKITD

SEQ ID NO: 54 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLKQHKITD

SEQ ID NO: 55 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLIQQKITD

SEQ ID NO: 56 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGSFTSELATILDXQAARDFIAWLIQYKITD

SEQ ID NO: 57 - protein, 33 aa, Artificial Sequence
  GLP-1/GLP-2 Dual agonist peptide
  MOD_RES (2)..(2): Aib
  MISC_FEATURE (16)..(16): Xaa is a residue of K, k (D-lys), R, Orn, Dap or Dab in which the side chain is conjugated to a substituent
HXEGTFSSELSTILEXQASREFIAWLIAYKITE

SEQ ID NO: 58 - protein, 39 aa, Artificial Sequence
  GLP-1/GLP-2 Dual agonist peptide
  MOD_RES (2)..(2): Aib
  MISC_FEATURE (34)..(38): Xaa at positions 34-38 is D-Lys
  MISC_FEATURE (39)..(39): Xaa is a residue of K, k (D-lys), R, Orn, Dap or Dab in which the side chain is conjugated to a substituent
HXEGTFSSELATILDEQAARDFIAWLIAHKITDXXXXXX

SEQ ID NO: 59 - protein, 39 aa, Artificial Sequence
  GLP-1/GLP-2 Dual agonist peptide
  MOD_RES (2)..(2): Aib
  MISC_FEATURE (34)..(38): Xaa at positions 34-38 is D-Lys
  MISC_FEATURE (39)..(39): Xaa is a residue of K, k (D-lys), R, Orn, Dap or Dab in which the side chain is conjugated to a substituent
HXEGTFTSELATILDEQAARDFIAWLIAHKITDXXXXXX

SEQ ID NO: 60 - protein, 39 aa, Artificial Sequence
  GLP-1/GLP-2 Dual agonist peptide
  MOD_RES (2)..(2): Aib
  MISC_FEATURE (34)..(38): Xaa at positions 34-38 is D-Lys
  MISC_FEATURE (39)..(39): Xaa is a residue of K, k (D-lys), R, Orn, Dap or Dab in which the side chain is conjugated to a substituent
HXEGSFTSELATILDEQAARDFIAWLIEHKITDXXXXXX

SEQ ID NO: 61 - protein, 33 aa, Artificial Sequence
  GLP-1/GLP-2 Dual agonist peptide
  MOD_RES (2)..(2): Aib
  MISC_FEATURE (10)..(10): Xaa is a residue of K, k (D-lys), R, Orn, Dap or Dab in which the side chain is conjugated to a substituent
HXEGSFTSEXATILDEQAARDFIAWLIEHKITD

SEQ ID NO: 62 - protein, 33 aa, Artificial Sequence
  GLP-1/GLP-2 Dual agonist peptide
  MOD_RES (2)..(2): Aib
  MISC_FEATURE (17)..(17): Xaa is a residue of K, k (D-lys), R, Orn, Dap or Dab in which the side chain is conjugated to a substituent
HXEGSFTSELATILEGXAARDFIAWLIEHKITD

SEQ ID NO: 63 - protein, 33 aa, Artificial Sequence
  GLP-1/GLP-2 Dual agonist peptide
  MOD_RES (2)..(2): Aib
  MISC_FEATURE (20)..(20): Xaa is a residue of K, k (D-lys), R, Orn, Dap or Dab in which the side chain is conjugated to a substituent
HXEGSFTSELATILDEQAAXDFIAWLIEHKITD

SEQ ID NO: 64 - protein, 33 aa, Artificial Sequence
  GLP-1/GLP-2 Dual agonist peptide
  MOD_RES (2)..(2): Aib
  MISC_FEATURE (20)..(20): Xaa is a residue of K, k (D-lys), R, Orn, Dap or Dab in which the side chain is conjugated to a substituent
HXEGTFTSELATILDEQAAXDFIAWLIEHKITD

SEQ ID NO: 65 - protein, 33 aa, Artificial Sequence
  GLP-1/GLP-2 Dual agonist peptide
  MOD_RES (2)..(2): Aib
  MISC_FEATURE (10)..(10): Xaa is a residue of K, k (D-lys), R, Orn, Dap or Dab in which the side chain is conjugated to a substituent
HXEGTFTSEXATILDEQAARDFIAWLIEHKITD

SEQ ID NO: 66 - protein, 33 aa, Artificial Sequence
  GLP-1/GLP-2 Dual agonist peptide
  MOD_RES (2)..(2): Aib
  MISC_FEATURE (17)..(17): Xaa is a residue of K, k (D-lys), R, Orn, Dap or Dab in which the side chain is conjugated to a substituent
HXEGSFTSELATILDAXAARDFIAWLIEHKITD

SEQ ID NO: 67 - protein, 33 aa, Artificial Sequence
  GLP-1/GLP-2 Dual agonist peptide
  MOD_RES (2)..(2): Aib
  MISC_FEATURE (20)..(20): Xaa is a residue of K, k (D-lys), R, Orn, Dap or Dab in which the side chain is conjugated to a substituent
HXEGSFTSELATILDAKAAXDFIAWLIEHKITD

SEQ ID NO: 68 - protein, 33 aa, Artificial Sequence; identical to SEQ ID NO: 21
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 69 - protein, 33 aa, Artificial Sequence; identical to SEQ ID NO: 22
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 70 - protein, 33 aa, Artificial Sequence; identical to SEQ ID NO: 23
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 71 - protein, 33 aa, Artificial Sequence; identical to SEQ ID NO: 24
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 72 - protein, 33 aa, Artificial Sequence; identical to SEQ ID NO: 25
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 73 - protein, 33 aa, Artificial Sequence; identical to SEQ ID NO: 26
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 74 - protein, 33 aa, Artificial Sequence; identical to SEQ ID NO: 27
  GLP-1/GLP-2 Dual agonist peptide
  MOD_RES (2)..(2): Aib
  MISC_FEATURE (17)..(17): Xaa indicates an L-lysine residue in which the side chain is conjugated to the substituent Z1- or Z1Z2

SEQ ID NO: 75 - protein, 33 aa, Artificial Sequence; identical to SEQ ID NO: 28
  GLP-1/GLP-2 Dual agonist peptide
  MOD_RES (2)..(2): Aib
  MISC_FEATURE (17)..(17): Xaa indicates an L-lysine residue in which the side chain is conjugated to the substituent Z1- or Z1Z2

SEQ ID NO: 76 - protein, 33 aa, Artificial Sequence; identical to SEQ ID NO: 29
  GLP-1/GLP-2 Dual agonist peptide
  MOD_RES (2)..(2): Aib
  MISC_FEATURE (17)..(17): Xaa indicates an L-lysine residue in which the side chain is conjugated to the substituent Z1- or Z1Z2

SEQ ID NO: 77 - protein, 33 aa, Artificial Sequence; identical to SEQ ID NO: 30
  GLP-1/GLP-2 Dual agonist peptide
  MOD_RES (2)..(2): Aib
  misc_feature (16)..(16): Xaa can be any naturally occurring amino acid
  MISC_FEATURE (17)..(17): Xaa indicates an L-lysine residue in which the side chain is conjugated to the substituent Z1- or Z1Z2

SEQ ID NO: 78 - protein, 33 aa, Artificial Sequence; identical to SEQ ID NO: 31
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 79 - protein, 33 aa, Artificial Sequence; identical to SEQ ID NO: 32
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 80 - protein, 33 aa, Artificial Sequence; identical to SEQ ID NO: 33
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 81 - protein, 33 aa, Artificial Sequence; identical to SEQ ID NO: 34
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 82 - protein, 33 aa, Artificial Sequence; identical to SEQ ID NO: 35
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 83 - protein, 33 aa, Artificial Sequence; identical to SEQ ID NO: 36
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 84 - protein, 33 aa, Artificial Sequence; identical to SEQ ID NO: 37
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 85 - protein, 33 aa, Artificial Sequence; identical to SEQ ID NO: 38
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 86 - protein, 33 aa, Artificial Sequence; identical to SEQ ID NO: 39
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 87 - protein, 33 aa, Artificial Sequence; identical to SEQ ID NO: 40
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 88 - protein, 33 aa, Artificial Sequence; identical to SEQ ID NO: 41
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 89 - protein, 33 aa, Artificial Sequence; identical to SEQ ID NO: 42
  GLP-1/GLP-2 Dual agonist peptide
  MOD_RES (2)..(2): Aib
  MISC_FEATURE (16)..(16): Xaa indicates an L-lysine residue respectively in which the side chain is conjugated to the substituent Z1- or Z1Z2
  MISC_FEATURE (16)..(16): Xaa indicates an L-lysine residue in which the side chain is conjugated to the substituent Z1- or Z1Z2

SEQ ID NO: 90 - protein, 33 aa, Artificial Sequence; identical to SEQ ID NO: 43
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 91 - protein, 33 aa, Artificial Sequence; identical to SEQ ID NO: 44
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 92 - protein, 33 aa, Artificial Sequence; identical to SEQ ID NO: 45
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 93 - protein, 33 aa, Artificial Sequence; identical to SEQ ID NO: 46
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 94 - protein, 33 aa, Artificial Sequence; identical to SEQ ID NO: 47
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 95 - protein, 33 aa, Artificial Sequence; identical to SEQ ID NO: 48
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 96 - protein, 33 aa, Artificial Sequence; identical to SEQ ID NO: 49
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 97 - protein, 33 aa, Artificial Sequence; identical to SEQ ID NO: 50
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 98 - protein, 33 aa, Artificial Sequence; identical to SEQ ID NO: 51
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 99 - protein, 33 aa, Artificial Sequence; identical to SEQ ID NO: 52
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 100 - protein, 33 aa, Artificial Sequence; identical to SEQ ID NO: 53
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 101 - protein, 33 aa, Artificial Sequence; identical to SEQ ID NO: 54
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 102 - protein, 33 aa, Artificial Sequence; identical to SEQ ID NO: 55
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 103 - protein, 33 aa, Artificial Sequence; identical to SEQ ID NO: 56
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 104 - protein, 33 aa, Artificial Sequence; identical to SEQ ID NO: 57
  GLP-1/GLP-2 Dual agonist peptide
  MOD_RES (2)..(2): Aib
  MISC_FEATURE (16)..(16): Xaa indicates an L-lysine residue in which the side chain is conjugated to the substituent Z1- or Z1Z2

SEQ ID NO: 105 - protein, 39 aa, Artificial Sequence; identical to SEQ ID NO: 58
  GLP-1/GLP-2 Dual agonist peptide
  MOD_RES (2)..(2): Aib
  MISC_FEATURE (34)..(38): Xaa at positions 34-38 is D-Lys
  MISC_FEATURE (39)..(39): Xaa indicates an D-lysine residue in which the side chain is conjugated to the substituent Z1- or Z1Z2

SEQ ID NO: 106 - protein, 39 aa, Artificial Sequence; identical to SEQ ID NO: 59
  GLP-1/GLP-2 Dual agonist peptide
  MOD_RES (2)..(2): Aib
  MISC_FEATURE (34)..(38): Xaa at positions 34-38 is D-Lys
  MISC_FEATURE (39)..(39): Xaa indicates an D-lysine residue in which the side chain is conjugated to the substituent Z1- or Z1Z2

SEQ ID NO: 107 - protein, 39 aa, Artificial Sequence; identical to SEQ ID NO: 60
  GLP-1/GLP-2 Dual agonist peptide
  MOD_RES (2)..(2): Aib
  MISC_FEATURE (34)..(38): Xaa at positions 34-38 is D-Lys
  MISC_FEATURE (39)..(39): Xaa indicates an D-lysine residue in which the side chain is conjugated to the substituent Z1- or Z1Z2

SEQ ID NO: 108 - protein, 33 aa, Artificial Sequence; identical to SEQ ID NO: 61
  GLP-1/GLP-2 Dual agonist peptide
  MOD_RES (2)..(2): Aib
  MISC_FEATURE (10)..(10): Xaa indicates an L-lysine residue in which the side chain is conjugated to the substituent Z1- or Z1Z2

SEQ ID NO: 109 - protein, 33 aa, Artificial Sequence; identical to SEQ ID NO: 62
  GLP-1/GLP-2 Dual agonist peptide
  MOD_RES (2)..(2): Aib
  MISC_FEATURE (17)..(17): Xaa indicates an L-lysine residue in which the side chain is conjugated to the substituent Z1- or Z1Z2

SEQ ID NO: 110 - protein, 33 aa, Artificial Sequence; identical to SEQ ID NO: 63
  GLP-1/GLP-2 Dual agonist peptide
  MOD_RES (2)..(2): Aib
  MISC_FEATURE (20)..(20): Xaa indicates an L-lysine residue in which the side chain is conjugated to the substituent Z1- or Z1Z2

SEQ ID NO: 111 - protein, 33 aa, Artificial Sequence; identical to SEQ ID NO: 64
  GLP-1/GLP-2 Dual agonist peptide
  MOD_RES (2)..(2): Aib
  MISC_FEATURE (20)..(20): Xaa indicates an L-lysine residue in which the side chain is conjugated to the substituent Z1- or Z1Z2

SEQ ID NO: 112 - protein, 33 aa, Artificial Sequence; identical to SEQ ID NO: 65
  GLP-1/GLP-2 Dual agonist peptide
  MOD_RES (2)..(2): Aib
  MISC_FEATURE (10)..(10): Xaa indicates an L-lysine residue in which the side chain is conjugated to the substituent Z1- or Z1Z2

SEQ ID NO: 113 - protein, 33 aa, Artificial Sequence; identical to SEQ ID NO: 66
  GLP-1/GLP-2 Dual agonist peptide
  MOD_RES (2)..(2): Aib
  MISC_FEATURE (17)..(17): Xaa indicates an L-lysine residue in which the side chain is conjugated to the substituent Z1- or Z1Z2

SEQ ID NO: 114 - protein, 33 aa, Artificial Sequence; identical to SEQ ID NO: 67
  GLP-1/GLP-2 Dual agonist peptide
  MOD_RES (2)..(2): Aib
  MISC_FEATURE (20)..(20): Xaa indicates an L-lysine residue in which the side chain is conjugated to the substituent Z1- or Z1Z2

SEQ ID NO: 115 - protein, 33 aa, Artificial Sequence; identical to SEQ ID NO: 21
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 116 - protein, 33 aa, Artificial Sequence; identical to SEQ ID NO: 22
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 117 - protein, 33 aa, Artificial Sequence; identical to SEQ ID NO: 23
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 118 - protein, 33 aa, Artificial Sequence; identical to SEQ ID NO: 24
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 119 - protein, 33 aa, Artificial Sequence; identical to SEQ ID NO: 25
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 120 - protein, 33 aa, Artificial Sequence; identical to SEQ ID NO: 26
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 121 - protein, 33 aa, Artificial Sequence; identical to SEQ ID NO: 27
  GLP-1/GLP-2 Dual agonist peptide
  MOD_RES (2)..(2): Aib
  MISC_FEATURE (17)..(17): Xaa is K([17-carboxy-heptadecanoyl]-isoGlu)

SEQ ID NO: 122 - protein, 33 aa, Artificial Sequence; identical to SEQ ID NO: 28
  GLP-1/GLP-2 Dual agonist peptide
  MOD_RES (2)..(2): Aib
  MISC_FEATURE (17)..(17): Xaa is K([17-carboxy-heptadecanoyl]-isoGlu)

SEQ ID NO: 123 - protein, 33 aa, Artificial Sequence; identical to SEQ ID NO: 29
  GLP-1/GLP-2 Dual agonist peptide
  MOD_RES (2)..(2): Aib
  MISC_FEATURE (17)..(17): Xaa  is K([17-carboxy-heptadecanoyl]-isoGlu)

SEQ ID NO: 124 - protein, 33 aa, Artificial Sequence; identical to SEQ ID NO: 30
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 125 - protein, 33 aa, Artificial Sequence; identical to SEQ ID NO: 31
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 126 - protein, 33 aa, Artificial Sequence; identical to SEQ ID NO: 32
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 127 - protein, 33 aa, Artificial Sequence; identical to SEQ ID NO: 33
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 128 - protein, 33 aa, Artificial Sequence; identical to SEQ ID NO: 34
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 129 - protein, 33 aa, Artificial Sequence; identical to SEQ ID NO: 35
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 130 - protein, 33 aa, Artificial Sequence; identical to SEQ ID NO: 36
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 131 - protein, 33 aa, Artificial Sequence; identical to SEQ ID NO: 37
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 132 - protein, 33 aa, Artificial Sequence; identical to SEQ ID NO: 38
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 133 - protein, 33 aa, Artificial Sequence; identical to SEQ ID NO: 39
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 134 - protein, 33 aa, Artificial Sequence; identical to SEQ ID NO: 40
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 135 - protein, 33 aa, Artificial Sequence; identical to SEQ ID NO: 41
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 136 - protein, 33 aa, Artificial Sequence; identical to SEQ ID NO: 42
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 137 - protein, 33 aa, Artificial Sequence; identical to SEQ ID NO: 43
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 138 - protein, 33 aa, Artificial Sequence; identical to SEQ ID NO: 44
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 139 - protein, 33 aa, Artificial Sequence; identical to SEQ ID NO: 45
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 140 - protein, 33 aa, Artificial Sequence; identical to SEQ ID NO: 46
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 141 - protein, 33 aa, Artificial Sequence; identical to SEQ ID NO: 47
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 142 - protein, 33 aa, Artificial Sequence; identical to SEQ ID NO: 48
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 143 - protein, 33 aa, Artificial Sequence; identical to SEQ ID NO: 49
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 144 - protein, 33 aa, Artificial Sequence; identical to SEQ ID NO: 50
  GLP-1/GLP-2 Dual agonist peptide
  MOD_RES (2)..(2): Aib
  MISC_FEATURE (16)..(16): Xaa is K([17-carboxy-heptadecanoyl]-isoGlu)

SEQ ID NO: 145 - protein, 33 aa, Artificial Sequence; identical to SEQ ID NO: 51
  GLP-1/GLP-2 Dual agonist peptide
  MOD_RES (2)..(2): Aib
  misc (16)..(16): Xaa is K([17-carboxy-heptadecanoyl]-isoGlu)

SEQ ID NO: 146 - protein, 33 aa, Artificial Sequence; identical to SEQ ID NO: 52
  GLP-1/GLP-2 Dual agonist peptide
  MOD_RES (2)..(2): Aib
  MISC_FEATURE (16)..(16): Xaa is K([Hexadecanoyl]- betaAla)

SEQ ID NO: 147 - protein, 33 aa, Artificial Sequence; identical to SEQ ID NO: 53
  GLP-1/GLP-2 Dual agonist peptide
  MOD_RES (2)..(2): Aib
  MISC_FEATURE (16)..(16): Xaa is K([17-carboxy-heptadecanoyl]iso-Glu- Peg3)

SEQ ID NO: 148 - protein, 33 aa, Artificial Sequence; identical to SEQ ID NO: 54
  GLP-1/GLP-2 Dual agonist peptide
  MOD_RES (2)..(2): Aib
  MISC_FEATURE (16)..(16): Xaa is K([19-carboxy-nonadecanoyl]iso-Glu-Peg3- Peg3)

SEQ ID NO: 149 - protein, 33 aa, Artificial Sequence; identical to SEQ ID NO: 55
  GLP-1/GLP-2 Dual agonist peptide
  MOD_RES (2)..(2): Aib
  MISC_FEATURE (16)..(16): Xaa is K([19-carboxy-nonadecanoyl]iso-Lys-Peg3- Peg3-Peg3)

SEQ ID NO: 150 - protein, 33 aa, Artificial Sequence; identical to SEQ ID NO: 56
  GLP-1/GLP-2 Dual agonist peptide
  MOD_RES (2)..(2): Aib
  MISC_FEATURE (16)..(16): Xaa is K(Octadecanoyl)

SEQ ID NO: 151 - protein, 33 aa, Artificial Sequence; identical to SEQ ID NO: 57
  GLP-1/GLP-2 Dual agonist peptide
  MOD_RES (2)..(2): Aib
  MISC_FEATURE (16)..(16): Xaa is K(Hexadecanoyl-isoGlu)

SEQ ID NO: 152 - protein, 40 aa, Artificial Sequence
  GLP-1/GLP-2 Dual agonist peptide
  MOD_RES (2)..(2): Aib
  MISC_FEATURE (34)..(39): Xaa at positions 34-39 is D-Lys
  MISC_FEATURE (40)..(40): Xaa is ([17-carboxy-Heptadecanoyl]-isoGlu)
HXEGTFSSELATILDEQAARDFIAWLIAHKITDXXXXXXX

SEQ ID NO: 153 - protein, 40 aa, Artificial Sequence
  GLP-1/GLP-2 Dual agonist peptide
  MOD_RES (2)..(2): Aib
  MISC_FEATURE (34)..(39): Xaa at positions 34-39 is D-Lys
  MISC_FEATURE (40)..(40): Xaa is ([17-carboxy-Heptadecanoyl]-isoGlu)
HXEGTFTSELATILDEQAARDFIAWLIAHKITDXXXXXXX

SEQ ID NO: 154 - protein, 40 aa, Artificial Sequence
  GLP-1/GLP-2 Dual agonist peptide
  MOD_RES (2)..(2): Aib
  MISC_FEATURE (34)..(39): Xaa at positions 34-39 is D-Lys
  MISC_FEATURE (40)..(40): Xaa is ([17-carboxy-Heptadecanoyl]-isoGlu)
HXEGSFTSELATILDEQAARDFIAWLIEHKITDXXXXXXX

SEQ ID NO: 155 - protein, 33 aa, Artificial Sequence; identical to SEQ ID NO: 39
  GLP-1/GLP-2 Dual agonist peptide
  MOD_RES (2)..(2): Aib
  MISC_FEATURE (16)..(16): Xaa is K([19-Carboxy-nonadecanoyl]-isoGlu)

SEQ ID NO: 156 - protein, 33 aa, Artificial Sequence; identical to SEQ ID NO: 61
  GLP-1/GLP-2 Dual agonist peptide
  MOD_RES (2)..(2): Aib
  MISC_FEATURE (10)..(10): Xaa is K([19-carboxy-nonadecanoyl]iso-Glu-Peg3- Peg3)

SEQ ID NO: 157 - protein, 33 aa, Artificial Sequence; identical to SEQ ID NO: 25
  GLP-1/GLP-2 Dual agonist peptide
  MOD_RES (2)..(2): Aib
  MISC_FEATURE (16)..(16): Xaa is K([19-carboxy-nonadecanoyl]iso-Glu-Peg3- Peg3)

SEQ ID NO: 158 - protein, 33 aa, Artificial Sequence; identical to SEQ ID NO: 62
  GLP-1/GLP-2 Dual agonist peptide
  MOD_RES (2)..(2): Aib
  MISC_FEATURE (17)..(17): Xaa is K([19-carboxy-nonadecanoyl]iso-Glu-Peg3- Peg3)

SEQ ID NO: 159 - protein, 33 aa, Artificial Sequence; identical to SEQ ID NO: 63
  GLP-1/GLP-2 Dual agonist peptide
  MOD_RES (2)..(2): Aib
  MISC_FEATURE (20)..(20): Xaa is K([19-carboxy-nonadecanoyl]iso-Glu-Peg3- Peg3)

SEQ ID NO: 160 - protein, 33 aa, Artificial Sequence; identical to SEQ ID NO: 64
  GLP-1/GLP-2 Dual agonist peptide
  MOD_RES (2)..(2): Aib
  MISC_FEATURE (20)..(20): Xaa is K([19-carboxy-nonadecanoyl]iso-Glu-Peg3- Peg3)

SEQ ID NO: 161 - protein, 33 aa, Artificial Sequence; identical to SEQ ID NO: 37
  GLP-1/GLP-2 Dual agonist peptide
  MOD_RES (2)..(2): Aib
  MISC_FEATURE (16)..(16): Xaa is K([17-Carboxy-heptadecanoyl]-isoGlu-KEK- Peg3)

SEQ ID NO: 162 - protein, 33 aa, Artificial Sequence; identical to SEQ ID NO: 37
  GLP-1/GLP-2 Dual agonist peptide
  MOD_RES (2)..(2): Aib
  MISC_FEATURE (16)..(16): Xaa is K([19-Carboxy-nonadecanoyl]-isoGlu-KEK- Peg3)

SEQ ID NO: 163 - protein, 33 aa, Artificial Sequence; identical to SEQ ID NO: 40
  GLP-1/GLP-2 Dual agonist peptide
  MOD_RES (2)..(2): Aib
  MISC_FEATURE (16)..(16): Xaa is K([17-Carboxy-heptadecanoyl]-isoGlu-KEK- Peg3)

SEQ ID NO: 164 - protein, 33 aa, Artificial Sequence; identical to SEQ ID NO: 40
  GLP-1/GLP-2 Dual agonist peptide
  MOD_RES (2)..(2): Aib
  MISC_FEATURE (16)..(16): Xaa is K([19-Carboxy-nonadecanoyl]-isoGlu-KEK- Peg3)

SEQ ID NO: 165 - protein, 33 aa, Artificial Sequence; identical to SEQ ID NO: 39
  GLP-1/GLP-2 Dual agonist peptide
  MOD_RES (2)..(2): Aib
  MISC_FEATURE (16)..(16): Xaa is K([19-Carboxy-nonadecanoyl]-isoGlu-KEK)

SEQ ID NO: 166 - protein, 33 aa, Artificial Sequence; identical to SEQ ID NO: 39
  GLP-1/GLP-2 Dual agonist peptide
  MOD_RES (2)..(2): Aib
  MISC_FEATURE (16)..(16): Xaa is K([19-Carboxy-nonadecanoyl]-isoGlu-KEK- Peg3)

SEQ ID NO: 167 - protein, 33 aa, Artificial Sequence; identical to SEQ ID NO: 61
  GLP-1/GLP-2 Dual agonist peptide
  MOD_RES (2)..(2): Aib
  MISC_FEATURE (10)..(10): Xaa is K([19-Carboxy-nonadecanoyl]-isoGlu-KEK- Peg3)

SEQ ID NO: 168 - protein, 33 aa, Artificial Sequence; identical to SEQ ID NO: 65
  GLP-1/GLP-2 Dual agonist peptide
  MOD_RES (2)..(2): Aib
  MISC_FEATURE (10)..(10): Xaa is K([19-Carboxy-nonadecanoyl]-isoGlu-KEK- Peg3)

SEQ ID NO: 169 - protein, 33 aa, Artificial Sequence; identical to SEQ ID NO: 61
  GLP-1/GLP-2 Dual agonist peptide
  MOD_RES (2)..(2): Aib
  MISC_FEATURE (10)..(10): Xaa is K([19-carboxy-nonadecanoyl]iso-Glu-KEK- Peg3-Peg3)

SEQ ID NO: 170 - protein, 33 aa, Artificial Sequence; identical to SEQ ID NO: 45
  GLP-1/GLP-2 Dual agonist peptide
  MOD_RES (2)..(2): Aib
  MISC_FEATURE (16)..(16): Xaa is K([19-Carboxy-nonadecanoyl]-isoGlu-KEK- Peg3)

SEQ ID NO: 171 - protein, 33 aa, Artificial Sequence; identical to SEQ ID NO: 44
  GLP-1/GLP-2 Dual agonist peptide
  MOD_RES (2)..(2): Aib
  MISC_FEATURE (16)..(16): Xaa is K([19-Carboxy-nonadecanoyl]-isoGlu-KEK- Peg3)

SEQ ID NO: 172 - protein, 33 aa, Artificial Sequence; identical to SEQ ID NO: 42
  GLP-1/GLP-2 Dual agonist peptide
  MOD_RES (2)..(2): Aib
  MISC_FEATURE (16)..(16): Xaa is K([19-Carboxy-nonadecanoyl]-isoGlu-KEK- Peg3)

SEQ ID NO: 173 - protein, 33 aa, Artificial Sequence; identical to SEQ ID NO: 25
  GLP-1/GLP-2 Dual agonist peptide
  MOD_RES (2)..(2): Aib
  MISC_FEATURE (16)..(16): Xaa is K([19-Carboxy-nonadecanoyl]-isoGlu-KEK- Peg3)

SEQ ID NO: 174 - protein, 33 aa, Artificial Sequence; identical to SEQ ID NO: 44
  GLP-1/GLP-2 Dual agonist peptide
  MOD_RES (2)..(2): Aib
  MISC_FEATURE (16)..(16): Xaa is K([19-carboxy-nonadecanoyl]iso-Glu-KEK- Peg3-Peg3)

SEQ ID NO: 175 - protein, 33 aa, Artificial Sequence; identical to SEQ ID NO: 62
  GLP-1/GLP-2 Dual agonist peptide
  MOD_RES (2)..(2): Aib
  MISC_FEATURE (17)..(17): Xaa is K([19-Carboxy-nonadecanoyl]-isoGlu-KEK- Peg3)

SEQ ID NO: 176 - protein, 33 aa, Artificial Sequence; identical to SEQ ID NO: 66
  GLP-1/GLP-2 Dual agonist peptide
  MOD_RES (2)..(2): Aib
  MISC_FEATURE (17)..(17): Xaa is K([19-Carboxy-nonadecanoyl]-isoGlu-KEK- Peg3)

SEQ ID NO: 177 - protein, 33 aa, Artificial Sequence; identical to SEQ ID NO: 66
  GLP-1/GLP-2 Dual agonist peptide
  MOD_RES (2)..(2): Aib
  MISC_FEATURE (17)..(17): Xaa is K([19-carboxy-nonadecanoyl]iso-Glu-KEK- Peg3-Peg3)

SEQ ID NO: 178 - protein, 33 aa, Artificial Sequence; identical to SEQ ID NO: 63
  GLP-1/GLP-2 Dual agonist peptide
  MOD_RES (2)..(2): Aib
  MISC_FEATURE (20)..(20): Xaa is K([19-Carboxy-nonadecanoyl]-isoGlu-KEK- Peg3)

SEQ ID NO: 179 - protein, 33 aa, Artificial Sequence; identical to SEQ ID NO: 64
  GLP-1/GLP-2 Dual agonist peptide
  MOD_RES (2)..(2): Aib
  MISC_FEATURE (20)..(20): Xaa is K([19-Carboxy-nonadecanoyl]-isoGlu-KEK- Peg3)

SEQ ID NO: 180 - protein, 33 aa, Artificial Sequence; identical to SEQ ID NO: 63
  GLP-1/GLP-2 Dual agonist peptide
  MOD_RES (2)..(2): Aib
  MISC_FEATURE (20)..(20): Xaa is K([19-carboxy-nonadecanoyl]iso-Glu-KEK- Peg3-Peg3)

SEQ ID NO: 181 - protein, 33 aa, Artificial Sequence; identical to SEQ ID NO: 64
  GLP-1/GLP-2 Dual agonist peptide
  MOD_RES (2)..(2): Aib
  MISC_FEATURE (20)..(20): Xaa is K([19-carboxy-nonadecanoyl]iso-Glu-KEK- Peg3-Peg3)

SEQ ID NO: 182 - protein, 33 aa, Artificial Sequence; identical to SEQ ID NO: 67
  GLP-1/GLP-2 Dual agonist peptide
  MOD_RES (2)..(2): Aib
  MISC_FEATURE (20)..(20): Xaa is K([19-Carboxy-nonadecanoyl]-isoGlu-KEK- Peg3)

SEQ ID NO: 183 - protein, 4 aa, Artificial Sequence
  GLP-1/GLP-2 Dual agonist peptide
  MISC_FEATURE (1)..(1): Xaa is betaAla
XKEK

SEQ ID NO: 184 - protein, 4 aa, Artificial Sequence
  GLP-1/GLP-2 Dual agonist peptide
  MISC_FEATURE (1)..(1): Xaa is isoLys
XKEK

SEQ ID NO: 185 - protein, 4 aa, Artificial Sequence
  GLP-1/GLP-2 Dual agonist peptide
  MISC_FEATURE (4)..(4): Xaa is isoGlu
KEKX

SEQ ID NO: 186 - protein, 4 aa, Artificial Sequence
  GLP-1/GLP-2 Dual agonist peptide
  MISC_FEATURE (4)..(4): Xaa is betaAla
KEKX

SEQ ID NO: 187 - protein, 4 aa, Artificial Sequence
  GLP-1/GLP-2 Dual agonist peptide
  MISC_FEATURE (4)..(4): Xaa is isoLys
KEKX